SEQUENCE LISTING (287 sequences)

SEQ ID NO: 1 - DNA, 20 bp, synthetic construct
  misc_feature 1..20: note = Description of sequence: primer
  source 1..20: mol_type = other DNA
agagtttgatcctggctcag

SEQ ID NO: 2 - DNA, 17 bp, synthetic construct
  misc_feature 1..17: note = Description of sequence: primer
  source 1..17: mol_type = other DNA
attaccgcggctgctgg

SEQ ID NO: 3 - DNA, 30 bp, synthetic construct
  misc_feature 1..30: note = Description of sequence: primer
  source 1..30: mol_type = other DNA
ccatctcatccctgcgtgtctccgactcag

SEQ ID NO: 4 - DNA, 30 bp, synthetic construct
  misc_feature 1..30: note = Description of sequence: primer
  source 1..30: mol_type = other DNA
cctatcccctgtgtgccttggcagtctcag

SEQ ID NO: 5 - DNA, 16 bp, synthetic construct
  misc_feature 1..16: note = Description of sequence: Nucleic acid unwinding assay component
  source 1..16: mol_type = other DNA
  modified_base 1: mod_base = OTHER; note = bound to dye, Cyanine3
  modified_base 16: mod_base = OTHER; note = bound to quencher, blackhole quencher
gtgggcagctggccaa

SEQ ID NO: 6 - DNA, 16 bp, synthetic construct
  misc_feature 1..16: note = Description of sequence: Nucleic acid unwinding assay component
  source 1..16: mol_type = other DNA
  modified_base 1: mod_base = OTHER; note = bound to dye, Cyanine3
  modified_base 15: mod_base = OTHER; note = bound to quencher, blackhole quencher
gtatgcagctggataa

SEQ ID NO: 7 - DNA, 57 bp, synthetic construct
  misc_feature 1..57: note = Description of sequence: Oligonucleotide in S100A8 promoter. Name: S100p1
  source 1..57: mol_type = other DNA
ctgtgggcagctggccaagctttcctctataaaagcagctgacacttagcctcacat

SEQ ID NO: 8 - DNA, 20 bp, synthetic construct
  misc_feature 1..20: note = Description of sequence: Oligonucleotide in S100A8 promoter. Name: S100p1-G
  source 1..20: mol_type = other DNA
ctgtgggcagctggccaagc

SEQ ID NO: 9 - DNA, 20 bp, synthetic construct
  misc_feature 1..20: note = Description of sequence: Oligonucleotide in S100A8 promoter. Name: S100p1-G-Mut
  source 1..20: mol_type = other DNA
ctgtatgcaattggccaagc

SEQ ID NO: 10 - DNA, 14 bp, synthetic construct
  misc_feature 1..14: note = Description of sequence: Binding sequence: IAA sequestration agent, IAA binding sequence
  source 1..14: mol_type = other DNA
tgggcagctggcca

SEQ ID NO: 11 - DNA, 20 bp, synthetic construct
  misc_feature 1..20: note = Description of sequence: Binding sequence: IAA sequestration agent, IAA binding sequence
  source 1..20: mol_type = other DNA
ctgtgggcagctggccaagc

SEQ ID NO: 12 - DNA, 357 bp, Mus musculus
  misc_feature 1..357: note = Description of sequence: Murine S100A8 Promoter
  source 1..357: mol_type = genomic DNA
  misc_feature 304..317: note = IAA binding site
ttcagaggtagactggacatgaaggcattggatcagcaatggatccaattaggagagggttaagattgagagtctgtttagatgcagggatgaggtgccaggggcctagacatggacttattgccatgccccatcctgattcttcctgctgggtactcctgtctggtaaatgttccaacactcccacttcctcagactcagaaatgctcactgtactcagtgattgccacatggacttggttaggaaacagaggctgtggcaactctggaagggaagagcgttgtctccatagcccgaggctgtgggcagctggccaagctttcctctataaaagcagctgacacttagcctcacat

SEQ ID NO: 13 - DNA, 2001 bp, Homo sapiens
  misc_feature 1..2001: note = Description of sequence: Human S100A8 Promoter
  source 1..2001: mol_type = genomic DNA
  misc_feature 874..887: note = IAA binding site
tggggagaggatttgttcctcctgaaatcctggggaattggccacctcctcttctcctcttaggcatgaagcgcgtctggcttctccaaagaactcttcccctccactacctcagagttagcttcctctcttcagccagtgatcctggggtcccagacacaataattaaccaagagagggtgaaaggctccctgctgtgtttatgcaatggctcaggcccttgtgaagtgccgagggaccccaagcagcctccatctcccagggcatggtccatccccagcttcacagaacaggaaagctgtggaggagtgtgggcagcagggtaggaatggatatagcccttggcaacaacacatttccccacaaagcacccacccaaaagaacaacaacgatagttttagtttttagtaatgagaacaatagttctcatgactaaaagccatcagccaggacacgtgttctcaacccttttgcggtctttggaccctttgaaactctgacagaagccatggaggaatgttctcactgagtgcatgcactcaaaatgatgcattcaacttcaattcagtttcagggatgtatggcctgaccaccaatgcaggggattagcaatcgcaatagtggagagggcatgggagtgggaatctggctggatcaagcaagtggatgccagcagcccagaaaaagagcccccctacctgctttttccttctgggcactattgcccagcaaatgccttcctctttccgcttctcctacctccccacccaaaattttcattctgcacagtgattgccacattcacctggttgagaaaccagagactgtagcaactctggcagggagaagctgtctctgatggcctgaagctgtgggcagctggccaagcctaaccgctataaaaaggagctgcctctcagccctgcatgtctcttgtcagctgtctttcagaagacctggtaagtgggactgtctgggttggccccgcactttgggcttctcttggggagggtcagggaagtggagcagccttcctgagagaggagagagaaagctcagggaggtctggagcaaagatactcctggaggtggggagtgaggcagggataaggaaggagagtatcctccagcaccttccagtgggtaagggcacattgtctcctaggctggacttttcttgagcagagggtggggtggtaaggaaagtctacgggccccgtgtgtgtgcacatgtctctgtgtgaatggacccttccccttcccacacgtgtatccctatcatcccacccttcccaccagagccatagccatctgctggtttggttatttggagagtgcaggccaggacaaggccatcgcttggggcatgaatcctctgcgtactgccctggccagatgcaaattccctgccatgggattccccagaaggttctgtttttcaggtggggcaagtccgtgggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcagggtgaggaggggctgggtgtggcgggggctctctgcctggtcctggggctgccctgggccagcggtcctccctgccacccttcatagatggctatgcctcggctctctctgagatctttaaactctggcttcttcctcctcaatcttgacagaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtagctgagttactgggcccagaggctgggcccctggacatgtacctgcagaataataaagtcatcaatacctcatgcctctctcttatgcttttgtggaatgaggt

SEQ ID NO: 14 - DNA, 60 bp, synthetic construct
  misc_feature 1..60: note = Description of sequence: Oligonucleotide in S100A8 promoter. Name: S100p7
  source 1..60: mol_type = other DNA
ttcagaggtagactggacatgaagacattggatcagcaatggatccaattaggagagagc

SEQ ID NO: 15 - DNA, 60 bp, synthetic construct
  misc_feature 1..60: note = Description of sequence: Oligonucleotide in S100A8 promoter. Name: S100p6
  source 1..60: mol_type = other DNA
aggagagagctaagattgagagtctgtttagatgcagggatgaggtaccaggggcctaga

SEQ ID NO: 16 - DNA, 60 bp, synthetic construct
  misc_feature 1..60: note = Description of sequence: Oligonucleotide in S100A8 promoter. Name: S100p5
  source 1..60: mol_type = other DNA
ggggcctagacatggacttattgccatgccccatcctgattcttcctgctgggtactcct

SEQ ID NO: 17 - DNA, 60 bp, synthetic construct
  misc_feature 1..60: note = Description of sequence: Oligonucleotide in S100A8 promoter. Name: S100p4
  source 1..60: mol_type = other DNA
gggtactcctgtctggtaaatgttccaacactcccacttcctcagactcagaaatgctca

SEQ ID NO: 18 - DNA, 60 bp, synthetic construct
  misc_feature 1..60: note = Description of sequence: Oligonucleotide in S100A8 promoter. Name: S100p3
  source 1..60: mol_type = other DNA
gaaatgctcactgtactcagtgattgccacatggacttggttaggaaacagaggctgtgg

SEQ ID NO: 19 - DNA, 60 bp, synthetic construct
  misc_feature 1..60: note = Description of sequence: Oligonucleotide in S100A8 promoter. Name: S100p2
  source 1..60: mol_type = other DNA
gaggctgtggcaactctggaagggaagagcgttgtctccatagcccgaggctgtgggcag

SEQ ID NO: 20 - DNA, 10 bp, synthetic construct
  misc_feature 1..10: note = Description of sequence: Oligonucleotide in S100A8 promoter. Name: S100p1a
  source 1..10: mol_type = other DNA
cgaggctgtg

SEQ ID NO: 21 - DNA, 10 bp, synthetic construct
  misc_feature 1..10: note = Description of sequence: Oligonucleotide in S100A8 promoter. Name: S100p1b
  source 1..10: mol_type = other DNA
ctgtgggcag

SEQ ID NO: 22 - DNA, 10 bp, synthetic construct
  misc_feature 1..10: note = Description of sequence: Oligonucleotide in S100A8 promoter. Name: S100p1c
  source 1..10: mol_type = other DNA
ggcagctggc

SEQ ID NO: 23 - DNA, 10 bp, synthetic construct
  misc_feature 1..10: note = Description of sequence: Oligonucleotide in S100A8 promoter. Name: S100p1d
  source 1..10: mol_type = other DNA
ctggccaagc

SEQ ID NO: 24 - DNA, 10 bp, synthetic construct
  misc_feature 1..10: note = Description of sequence: Oligonucleotide in S100A8 promoter. Name: S100p1e
  source 1..10: mol_type = other DNA
caagctttcc

SEQ ID NO: 25 - DNA, 10 bp, synthetic construct
  misc_feature 1..10: note = Description of sequence: Oligonucleotide in S100A8 promoter. Name: S100p1f
  source 1..10: mol_type = other DNA
tttcctctat

SEQ ID NO: 26 - DNA, 10 bp, synthetic construct
  misc_feature 1..10: note = Description of sequence: Oligonucleotide in S100A8 promoter. Name: S100p1g
  source 1..10: mol_type = other DNA
tctataaaag

SEQ ID NO: 27 - DNA, 10 bp, synthetic construct
  misc_feature 1..10: note = Description of sequence: Oligonucleotide in S100A8 promoter. Name: S100p1h
  source 1..10: mol_type = other DNA
aaaagcagct

SEQ ID NO: 28 - DNA, 10 bp, synthetic construct
  misc_feature 1..10: note = Description of sequence: Oligonucleotide in S100A8 promoter. Name: S100p1i
  source 1..10: mol_type = other DNA
cagctgacac

SEQ ID NO: 29 - DNA, 10 bp, synthetic construct
  misc_feature 1..10: note = Description of sequence: Oligonucleotide in S100A8 promoter. Name: S100p1j
  source 1..10: mol_type = other DNA
gacacttagc

SEQ ID NO: 30 - DNA, 10 bp, synthetic construct
  misc_feature 1..10: note = Description of sequence: Oligonucleotide in S100A8 promoter. Name: S100p1k
  source 1..10: mol_type = other DNA
ttagcctcac

SEQ ID NO: 31 - DNA, 20 bp, synthetic construct
  misc_feature 1..20: note = Description of sequence: Oligonucleotide in S100A8 promoter. Name: S100p1-A
  source 1..20: mol_type = other DNA
aaaagcagctgacacttagc

SEQ ID NO: 32 - DNA, 20 bp, synthetic construct
  misc_feature 1..20: note = Description of sequence: Oligonucleotide in S100A8 promoter. Name: S100p1-B
  source 1..20: mol_type = other DNA
tctataaaagcagctgacac

SEQ ID NO: 33 - DNA, 20 bp, synthetic construct
  misc_feature 1..20: note = Description of sequence: Oligonucleotide in S100A8 promoter. Name: S100p1-C
  source 1..20: mol_type = other DNA
tttcctctataaaagcagct

SEQ ID NO: 34 - DNA, 20 bp, synthetic construct
  misc_feature 1..20: note = Description of sequence: Oligonucleotide in S100A8 promoter. Name: S100p1-D
  source 1..20: mol_type = other DNA
caagctttcctctataaaag

SEQ ID NO: 35 - DNA, 20 bp, synthetic construct
  misc_feature 1..20: note = Description of sequence: Oligonucleotide in S100A8 promoter. Name: S100p1-E
  source 1..20: mol_type = other DNA
ctggccaagctttcctctat

SEQ ID NO: 36 - DNA, 20 bp, synthetic construct
  misc_feature 1..20: note = Description of sequence: Oligonucleotide in S100A8 promoter. Name: S100p1-F
  source 1..20: mol_type = other DNA
ggcagctggccaagctttcc

SEQ ID NO: 37 - DNA, 20 bp, synthetic construct
  misc_feature 1..20: note = Description of sequence: Oligonucleotide in S100A8 promoter. Name: S100p1-H
  source 1..20: mol_type = other DNA
cgaggctgtgggcagctggc

SEQ ID NO: 38 - DNA, 20 bp, synthetic construct
  misc_feature 1..20: note = Description of sequence: Oligonucleotide in S100A8 promoter. Name: S100p1-I
  source 1..20: mol_type = other DNA
tagcccgaggctgtgggcag

SEQ ID NO: 39 - DNA, 26 bp, synthetic construct
  misc_feature 1..26: note = Description of sequence: Oligonucleotide in S100A8 promoter. Name: hS100A8p
  source 1..26: mol_type = other DNA
agctgtgggcagctggccaagcctaa

SEQ ID NO: 40 - DNA, 20 bp, synthetic construct
  misc_feature 1..20: note = Description of sequence: Primer. Direction: Forward primer, Name: P2ry12, Ligand: Nucleotides
  source 1..20: mol_type = other DNA
cacggattccctacaccctg

SEQ ID NO: 41 - DNA, 20 bp, synthetic construct
  misc_feature 1..20: note = Description of sequence: Primer. Direction: reverse primer, Name: P2ry12, Ligand: Nucleotides
  source 1..20: mol_type = other DNA
gggtgctctccttcacgtag

SEQ ID NO: 42 - DNA, 20 bp, synthetic construct
  misc_feature 1..20: note = Description of sequence: Primer. Direction: Forward primer, Name: P2ry13, Ligand: Nucleotides-ADP
  source 1..20: mol_type = other DNA
aacaaagctgatgctcggga

SEQ ID NO: 43 - DNA, 20 bp, synthetic construct
  misc_feature 1..20: note = Description of sequence: Primer. Direction: reverse primer, Name: P2ry13, Ligand: Nucleotides-ADP
  source 1..20: mol_type = other DNA
gtgtcatccgagtgtccctg

SEQ ID NO: 44 - DNA, 20 bp, synthetic construct
  misc_feature 1..20: note = Description of sequence: Primer. Direction: Forward primer, Name: P2ry6, Ligand: Nucleotides-UDP
  source 1..20: mol_type = other DNA
ctgcgtctaccgtgaggatt

SEQ ID NO: 45 - DNA, 20 bp, synthetic construct
  misc_feature 1..20: note = Description of sequence: Primer. Direction: reverse primer, Name: P2ry6, Ligand: Nucleotides-UDP
  source 1..20: mol_type = other DNA
gcaatgacgcagatgttcag

SEQ ID NO: 46 - DNA, 22 bp, synthetic construct
  misc_feature 1..22: note = Description of sequence: Primer. Direction: Forward primer, Name: Gpr34, Ligand: Nucleotides, LysoPS
  source 1..22: mol_type = other DNA
accggatggaagagcccagctg

SEQ ID NO: 47 - DNA, 24 bp, synthetic construct
  misc_feature 1..24: note = Description of sequence: Primer. Direction: reverse primer, Name: Gpr34, Ligand: Nucleotides, LysoPS
  source 1..24: mol_type = other DNA
cagtgtgacttctcaacgtcttct

SEQ ID NO: 48 - DNA, 20 bp, synthetic construct
  misc_feature 1..20: note = Description of sequence: Primer. Direction: Forward primer, Name: Adora3, Ligand: Adenosine
  source 1..20: mol_type = other DNA
ttgctggccattgctgtaga

SEQ ID NO: 49 - DNA, 25 bp, synthetic construct
  misc_feature 1..25: note = Description of sequence: Primer. Direction: reverse primer, Name: Adora3, Ligand: Adenosine
  source 1..25: mol_type = other DNA
gagtggtaaccgttctatatctgac

SEQ ID NO: 50 - DNA, 20 bp, synthetic construct
  misc_feature 1..20: note = Description of sequence: Primer. Direction: Forward primer, Name: Entpd1, Ligand: Nucleotides-ATP
  source 1..20: mol_type = other DNA
agctgccccttatggaagat

SEQ ID NO: 51 - DNA, 20 bp, synthetic construct
  misc_feature 1..20: note = Description of sequence: Primer. Direction: reverse primer, Name: Entpd1, Ligand: Nucleotides-ATP
  source 1..20: mol_type = other DNA
tcagtcccacagcaatcaaa

SEQ ID NO: 52 - DNA, 20 bp, synthetic construct
  misc_feature 1..20: note = Description of sequence: Primer. Direction: Forward primer, Name: Tmem173, Ligand: Bacterial Cyclic Di-GMP
  source 1..20: mol_type = other DNA
agcggaagtctctgcagtct

SEQ ID NO: 53 - DNA, 20 bp, synthetic construct
  misc_feature 1..20: note = Description of sequence: Primer. Direction: reverse primer, Name: Tmem173, Ligand: Bacterial Cyclic Di-GMP
  source 1..20: mol_type = other DNA
ggagccctggtaagatcaac

SEQ ID NO: 54 - DNA, 22 bp, synthetic construct
  misc_feature 1..22: note = Description of sequence: Primer. Direction: Forward primer, Name: Csf1r, Ligand: m-CSF, IL-34
  source 1..22: mol_type = other DNA
gcagtaccaccatccacttgta

SEQ ID NO: 55 - DNA, 22 bp, synthetic construct
  misc_feature 1..22: note = Description of sequence: Primer. Direction: reverse primer, Name: Csf1r, Ligand: m-CSF, IL-34
  source 1..22: mol_type = other DNA
gtgagacactgtccttcagtgc

SEQ ID NO: 56 - DNA, 20 bp, synthetic construct
  misc_feature 1..20: note = Description of sequence: Primer. Direction: Forward primer, Name: Csf3r, Ligand: g-CSF
  source 1..20: mol_type = other DNA
accctttgtgttccaccagt

SEQ ID NO: 57 - DNA, 20 bp, synthetic construct
  misc_feature 1..20: note = Description of sequence: Primer. Direction: reverse primer, Name: Csf3r, Ligand: g-CSF
  source 1..20: mol_type = other DNA
ttggtcctttcttcctccct

SEQ ID NO: 58 - DNA, 19 bp, synthetic construct
  misc_feature 1..19: note = Description of sequence: Primer. Direction: Forward primer, Name: Tgfbr1, Ligand: Tgf-beta
  source 1..19: mol_type = other DNA
tgccataaccgcactgtca

SEQ ID NO: 59 - DNA, 23 bp, synthetic construct
  misc_feature 1..23: note = Description of sequence: Primer. Direction: reverse primer, Name: Tgfbr1, Ligand: Tgf-beta
  source 1..23: mol_type = other DNA
aatgaaagggcgatctagtgatg

SEQ ID NO: 60 - DNA, 23 bp, synthetic construct
  misc_feature 1..23: note = Description of sequence: Primer. Direction: Forward primer, Name: Tgfbr2, Ligand: Tgf-beta
  source 1..23: mol_type = other DNA
cctactctgtctgtggatgacct

SEQ ID NO: 61 - DNA, 18 bp, synthetic construct
  misc_feature 1..18: note = Description of sequence: Primer. Direction: reverse primer, Name: Tgfbr2, Ligand: Tgf-beta
  source 1..18: mol_type = other DNA
acttccggggccatgtat

SEQ ID NO: 62 - DNA, 24 bp, synthetic construct
  misc_feature 1..24: note = Description of sequence: Primer. Direction: Forward primer, Name: Ifngr1, Ligand: Tgf-y
  source 1..24: mol_type = other DNA
gcactgggtggaatgagactattg

SEQ ID NO: 63 - DNA, 24 bp, synthetic construct
  misc_feature 1..24: note = Description of sequence: Primer. Direction: reverse primer, Name: Ifngr1, Ligand: Tgf-y
  source 1..24: mol_type = other DNA
gacctgtcagttgatgcctcagaa

SEQ ID NO: 64 - DNA, 21 bp, synthetic construct
  misc_feature 1..21: note = Description of sequence: Primer. Direction: Forward primer, Name: ll10ra, Ligand: IL-10
  source 1..21: mol_type = other DNA
caggaactgacggattgggaa

SEQ ID NO: 65 - DNA, 20 bp, synthetic construct
  misc_feature 1..20: note = Description of sequence: Primer. Direction: reverse primer, Name: ll10ra, Ligand: IL-10
  source 1..20: mol_type = other DNA
gcttcaaaccacacagacgg

SEQ ID NO: 66 - DNA, 21 bp, synthetic construct
  misc_feature 1..21: note = Description of sequence: Primer. Direction: Forward primer, Name: ll6ra, Ligand: IL-6
  source 1..21: mol_type = other DNA
cctctgacttccatttctgct

SEQ ID NO: 67 - DNA, 21 bp, synthetic construct
  misc_feature 1..21: note = Description of sequence: Primer. Direction: reverse primer, Name: ll6ra, Ligand: IL-6
  source 1..21: mol_type = other DNA
caagaatcctcgtccatgtcc

SEQ ID NO: 68 - DNA, 20 bp, synthetic construct
  misc_feature 1..20: note = Description of sequence: Primer. Direction: Forward primer, Name: ll21?, Ligand: IL-21
  source 1..20: mol_type = other DNA
ctccccccttgaacgtgact

SEQ ID NO: 69 - DNA, 20 bp, synthetic construct
  misc_feature 1..20: note = Description of sequence: Primer. Direction: reverse primer, Name: ll21?, Ligand: IL-21
  source 1..20: mol_type = other DNA
ttgcccctcagcacgtagtt

SEQ ID NO: 70 - DNA, 20 bp, synthetic construct
  misc_feature 1..20: note = Description of sequence: Primer. Direction: Forward primer, Name: Tnfrsf17, Ligand: BAFF
  source 1..20: mol_type = other DNA
gccgacaccgagctgactag

SEQ ID NO: 71 - DNA, 20 bp, synthetic construct
  misc_feature 1..20: note = Description of sequence: Primer. Direction: reverse primer, Name: Tnfrsf17, Ligand: BAFF
  source 1..20: mol_type = other DNA
cttgccgtagtcacccgttt

SEQ ID NO: 72 - DNA, 20 bp, synthetic construct
  misc_feature 1..20: note = Description of sequence: Primer. Direction: Forward primer, Name: Tnfrsf1b, Ligand: TNF
  source 1..20: mol_type = other DNA
gagatgccaaggtgcctcat

SEQ ID NO: 73 - DNA, 20 bp, synthetic construct
  misc_feature 1..20: note = Description of sequence: Primer. Direction: reverse primer, Name: Tnfrsf1b, Ligand: TNF
  source 1..20: mol_type = other DNA
aactgggtgctgtggtcaac

SEQ ID NO: 74 - DNA, 19 bp, synthetic construct
  misc_feature 1..19: note = Description of sequence: Primer. Direction: Forward primer, Name: Cx3cr1, Ligand: cx3cl1
  source 1..19: mol_type = other DNA
accggtaccttgccatcgt

SEQ ID NO: 75 - DNA, 19 bp, synthetic construct
  misc_feature 1..19: note = Description of sequence: Primer. Direction: reverse primer, Name: Cx3cr1, Ligand: cx3cl1
  source 1..19: mol_type = other DNA
acaccgtgctgcactgtcc

SEQ ID NO: 76 - DNA, 20 bp, synthetic construct
  misc_feature 1..20: note = Description of sequence: Primer. Direction: Forward primer, Name: Ccr5, Ligand: Ccl3, Ccl4, Ccl8, RANTES
  source 1..20: mol_type = other DNA
ggcagggctccgatgtataa

SEQ ID NO: 77 - DNA, 20 bp, synthetic construct
  misc_feature 1..20: note = Description of sequence: Primer. Direction: reverse primer, Name: Ccr5, Ligand: Ccl3, Ccl4, Ccl8, RANTES
  source 1..20: mol_type = other DNA
catccgttcccctacaagaa

SEQ ID NO: 78 - DNA, 23 bp, synthetic construct
  misc_feature 1..23: note = Description of sequence: Primer. Direction: Forward primer, Name: ????1, Ligand: C3a
  source 1..23: mol_type = other DNA
caccatggagtctttcgatgctg

SEQ ID NO: 79 - DNA, 19 bp, synthetic construct
  misc_feature 1..19: note = Description of sequence: Primer. Direction: reverse primer, Name: ????1, Ligand: C3a
  source 1..19: mol_type = other DNA
cacatctgtactcatattg

SEQ ID NO: 80 - DNA, 20 bp, synthetic construct
  misc_feature 1..20: note = Description of sequence: Primer. Direction: Forward primer, Name: Ptafr, Ligand: PAF
  source 1..20: mol_type = other DNA
cgagggcgactggattctac

SEQ ID NO: 81 - DNA, 20 bp, synthetic construct
  misc_feature 1..20: note = Description of sequence: Primer. Direction: reverse primer, Name: Ptafr, Ligand: PAF
  source 1..20: mol_type = other DNA
cacccaaaaaggccacactg

SEQ ID NO: 82 - DNA, 22 bp, synthetic construct
  misc_feature 1..22: note = Description of sequence: Primer. Direction: Forward primer, Name: Gpr77, Ligand: C5a
  source 1..22: mol_type = other DNA
cacaccaccagcgagtattatg

SEQ ID NO: 83 - DNA, 21 bp, synthetic construct
  misc_feature 1..21: note = Description of sequence: Primer. Direction: reverse primer, Name: Gpr77, Ligand: C5a
  source 1..21: mol_type = other DNA
agcacaagcaggactatcagg

SEQ ID NO: 84 - DNA, 22 bp, synthetic construct
  misc_feature 1..22: note = Description of sequence: Primer. Direction: Forward primer, Name: Cmklr1, Ligand: The chemokine chermin
  source 1..22: mol_type = other DNA
gctttggctactttgtggactt

SEQ ID NO: 85 - DNA, 23 bp, synthetic construct
  misc_feature 1..23: note = Description of sequence: Primer. Direction: reverse primer, Name: Cmklr1, Ligand: The chemokine chermin
  source 1..23: mol_type = other DNA
cagtgttcacggtcttcttcatc

SEQ ID NO: 86 - DNA, 20 bp, synthetic construct
  misc_feature 1..20: note = Description of sequence: Primer. Direction: Forward primer, Name: Cysltr1, Ligand: LTD4
  source 1..20: mol_type = other DNA
ctccaaggcaccaagcagac

SEQ ID NO: 87 - DNA, 21 bp, synthetic construct
  misc_feature 1..21: note = Description of sequence: Primer. Direction: reverse primer, Name: Cysltr1, Ligand: LTD4
  source 1..21: mol_type = other DNA
tgccaaagaaacccacaacag

SEQ ID NO: 88 - DNA, 20 bp, synthetic construct
  misc_feature 1..20: note = Description of sequence: Primer. Direction: Forward primer, Name: Ccrl2, Ligand: Chemerin
  source 1..20: mol_type = other DNA
tgtgtttcctgcttcccctg

SEQ ID NO: 89 - DNA, 20 bp, synthetic construct
  misc_feature 1..20: note = Description of sequence: Primer. Direction: reverse primer, Name: Ccrl2, Ligand: Chemerin
  source 1..20: mol_type = other DNA
cgaggagtggagtccgacaa

SEQ ID NO: 90 - DNA, 26 bp, synthetic construct
  misc_feature 1..26: note = Description of sequence: Primer. Direction: Forward primer, Name: Cmtm6, Ligand: May bind cxcl7
  source 1..26: mol_type = other DNA
ctagtattcttaattccttctgagcc

SEQ ID NO: 91 - DNA, 24 bp, synthetic construct
  misc_feature 1..24: note = Description of sequence: Primer. Direction: reverse primer, Name: Cmtm6, Ligand: May bind cxcl7
  source 1..24: mol_type = other DNA
agctttggctcagaaggaattaag

SEQ ID NO: 92 - DNA, 20 bp, synthetic construct
  misc_feature 1..20: note = Description of sequence: Primer. Direction: Forward primer, Name: C5ar1, Ligand: AKA CD88 C5a
  source 1..20: mol_type = other DNA
ggccatcctgcggctgatgg

SEQ ID NO: 93 - DNA, 20 bp, synthetic construct
  misc_feature 1..20: note = Description of sequence: Primer. Direction: reverse primer, Name: C5ar1, Ligand: AKA CD88 C5a
  source 1..20: mol_type = other DNA
gccttgcgactccaggtccg

SEQ ID NO: 94 - DNA, 25 bp, synthetic construct
  misc_feature 1..25: note = Description of sequence: Primer. Direction: Forward primer, Name: Fcgr3, Ligand: Fc portion of lgG
  source 1..25: mol_type = other DNA
cctccatctctctagtctggtacca

SEQ ID NO: 95 - DNA, 17 bp, synthetic construct
  misc_feature 1..17: note = Description of sequence: Primer. Direction: reverse primer, Name: Fcgr3, Ligand: Fc portion of lgG
  source 1..17: mol_type = other DNA
aggcccgtgtccactgc

SEQ ID NO: 96 - DNA, 20 bp, synthetic construct
  misc_feature 1..20: note = Description of sequence: Primer. Direction: Forward primer, Name: Fcer1g, Ligand: Fc portion of lgE
  source 1..20: mol_type = other DNA
aagatccaggtccgaaaggc

SEQ ID NO: 97 - DNA, 20 bp, synthetic construct
  misc_feature 1..20: note = Description of sequence: Primer. Direction: reverse primer, Name: Fcer1g, Ligand: Fc portion of lgE
  source 1..20: mol_type = other DNA
tctgaagctactggggtggt

SEQ ID NO: 98 - DNA, 22 bp, synthetic construct
  misc_feature 1..22: note = Description of sequence: Primer. Direction: Forward primer, Name: Fcgr2b, Ligand: Fc portion of lgG-Low affinity
  source 1..22: mol_type = other DNA
gaaaccatcacgctaaggtgcc

SEQ ID NO: 99 - DNA, 22 bp, synthetic construct
  misc_feature 1..22: note = Description of sequence: Primer. Direction: reverse primer, Name: Fcgr2b, Ligand: Fc portion of lgG-Low affinity
  source 1..22: mol_type = other DNA
tggtgcagtgtccttcctagac

SEQ ID NO: 100 - DNA, 24 bp, synthetic construct
  misc_feature 1..24: note = Description of sequence: Primer. Direction: Forward primer, Name: Fcgr1, Ligand: Fc portion of lgG-high affinity
  source 1..24: mol_type = other DNA
gcggaaagagaagatgctggattc

SEQ ID NO: 101 - DNA, 22 bp, synthetic construct
  misc_feature 1..22: note = Description of sequence: Primer. Direction: reverse primer, Name: Fcgr1, Ligand: Fc portion of lgG-high affinity
  source 1..22: mol_type = other DNA
cttctctctctgcagcctgtgt

SEQ ID NO: 102 - DNA, 20 bp, synthetic construct
  misc_feature 1..20: note = Description of sequence: Primer. Direction: Forward primer, Name: Cmtm7, Ligand: lgM
  source 1..20: mol_type = other DNA
actatggtgcccacagcttc

SEQ ID NO: 103 - DNA, 20 bp, synthetic construct
  misc_feature 1..20: note = Description of sequence: Primer. Direction: reverse primer, Name: Cmtm7, Ligand: lgM
  source 1..20: mol_type = other DNA
aacccaaagatcgacagggg

SEQ ID NO: 104 - DNA, 21 bp, synthetic construct
  misc_feature 1..21: note = Description of sequence: Primer. Direction: Forward primer, Name: Fcrl1, Ligand: lgG3
  source 1..21: mol_type = other DNA
cacacggagtaagtgagtcct

SEQ ID NO: 105 - DNA, 20 bp, synthetic construct
  misc_feature 1..20: note = Description of sequence: Primer. Direction: reverse primer, Name: Fcrl1, Ligand: lgG3
  source 1..20: mol_type = other DNA
tcaggccttgggcttgtatg

SEQ ID NO: 106 - DNA, 20 bp, synthetic construct
  misc_feature 1..20: note = Description of sequence: Primer. Direction: Forward primer, Name: Fcgr4, Ligand: Binds Fc fragment of lgG
  source 1..20: mol_type = other DNA
tccaccgtggcatcaaatca

SEQ ID NO: 107 - DNA, 20 bp, synthetic construct
  misc_feature 1..20: note = Description of sequence: Primer. Direction: reverse primer, Name: Fcgr4, Ligand: Binds Fc fragment of lgG
  source 1..20: mol_type = other DNA
gtcctgaggttccttgctcc

SEQ ID NO: 108 - DNA, 21 bp, synthetic construct
  misc_feature 1..21: note = Description of sequence: Primer. Direction: Forward primer, Name: Selplg, Ligand: Enterovirus 71, CD162
  source 1..21: mol_type = other DNA
gaaagggctgattgtgacccc

SEQ ID NO: 109 - DNA, 21 bp, synthetic construct
  misc_feature 1..21: note = Description of sequence: Primer. Direction: reverse primer, Name: Selplg, Ligand: Enterovirus 71, CD162
  source 1..21: mol_type = other DNA
agtagttccgcactgggtaca

SEQ ID NO: 110 - DNA, 21 bp, synthetic construct
  misc_feature 1..21: note = Description of sequence: Primer. Direction: Forward primer, Name: Ly86, Ligand: AKA MD1 LPS
  source 1..21: mol_type = other DNA
ctgccctccttgtgtggattc

SEQ ID NO: 111 - DNA, 22 bp, synthetic construct
  misc_feature 1..22: note = Description of sequence: Primer. Direction: reverse primer, Name: Ly86, Ligand: AKA MD1 LPS
  source 1..22: mol_type = other DNA
tggaacactggtcaatggaaag

SEQ ID NO: 112 - DNA, 20 bp, synthetic construct
  misc_feature 1..20: note = Description of sequence: Primer. Direction: Forward primer, Name: Cd68, Ligand: Ox LDL SR
  source 1..20: mol_type = other DNA
actggtgtagcctagctggt

SEQ ID NO: 113 - DNA, 20 bp, synthetic construct
  misc_feature 1..20: note = Description of sequence: Primer. Direction: reverse primer, Name: Cd68, Ligand: Ox LDL SR
  source 1..20: mol_type = other DNA
ccttgggctataagcggtcc

SEQ ID NO: 114 - DNA, 20 bp, synthetic construct
  misc_feature 1..20: note = Description of sequence: Primer. Direction: Forward primer, Name: Trem2, Ligand: Apoptotic neurons, Lipids, Abeta
  source 1..20: mol_type = other DNA
gccttcctgaagaagcggaa

SEQ ID NO: 115 - DNA, 20 bp, synthetic construct
  misc_feature 1..20: note = Description of sequence: Primer. Direction: reverse primer, Name: Trem2, Ligand: Apoptotic neurons, Lipids, Abeta
  source 1..20: mol_type = other DNA
gagtgatggtgacggttcca

SEQ ID NO: 116 - DNA, 23 bp, synthetic construct
  misc_feature 1..23: note = Description of sequence: Primer. Direction: Forward primer, Name: Cd180, Ligand: LPS AKA rp 105
  source 1..23: mol_type = other DNA
taggtctcaatgaaattcctggc

SEQ ID NO: 117 - DNA, 22 bp, synthetic construct
  misc_feature 1..22: note = Description of sequence: Primer. Direction: reverse primer, Name: Cd180, Ligand: LPS AKA rp 105
  source 1..22: mol_type = other DNA
aatctggcacctggttaaatcc

SEQ ID NO: 118 - DNA, 20 bp, synthetic construct
  misc_feature 1..20: note = Description of sequence: Primer. Direction: Forward primer, Name: Tlr2, Ligand: Microbial
  source 1..20: mol_type = other DNA
gcaaacgctgttctgctcag

SEQ ID NO: 119 - DNA, 22 bp, synthetic construct
  misc_feature 1..22: note = Description of sequence: Primer. Direction: reverse primer, Name: Tlr2, Ligand: Microbial
  source 1..22: mol_type = other DNA
aggcgtctccctctattgtatt

SEQ ID NO: 120 - DNA, 20 bp, synthetic construct
  misc_feature 1..20: note = Description of sequence: Primer. Direction: Forward primer, Name: Cd37, Ligand: P-glucan, dectin-1
  source 1..20: mol_type = other DNA
gcccaagagagttgcctcag

SEQ ID NO: 121 - DNA, 22 bp, synthetic construct
  misc_feature 1..22: note = Description of sequence: Primer. Direction: reverse primer, Name: Cd37, Ligand: P-glucan, dectin-1
  source 1..22: mol_type = other DNA
ggccgcctagtacaaagaagaa

SEQ ID NO: 122 - DNA, 21 bp, synthetic construct
  misc_feature 1..21: note = Description of sequence: Primer. Direction: Forward primer, Name: Tlr7, Ligand: Pattern recognition receptor
  source 1..21: mol_type = other DNA
atgtggacacggaagagacaa

SEQ ID NO: 123 - DNA, 22 bp, synthetic construct
  misc_feature 1..22: note = Description of sequence: Primer. Direction: reverse primer, Name: Tlr7, Ligand: Pattern recognition receptor
  source 1..22: mol_type = other DNA
ggtaagggtaagattggtggtg

SEQ ID NO: 124 - DNA, 22 bp, synthetic construct
  misc_feature 1..22: note = Description of sequence: Primer. Direction: Forward primer, Name: Cd14, Ligand: Pattern recognition receptor
  source 1..22: mol_type = other DNA
ctctgtccttaaagcggcttac

SEQ ID NO: 125 - DNA, 21 bp, synthetic construct
  misc_feature 1..21: note = Description of sequence: Primer. Direction: reverse primer, Name: Cd14, Ligand: Pattern recognition receptor
  source 1..21: mol_type = other DNA
gttgcggaggttcaagatgtt

SEQ ID NO: 126 - DNA, 23 bp, synthetic construct
  misc_feature 1..23: note = Description of sequence: Primer. Direction: Forward primer, Name: Cleo4a3, Ligand: HIV AKA DCIR3
  source 1..23: mol_type = other DNA
cttcacttcaactgacttggtgg

SEQ ID NO: 127 - DNA, 21 bp, synthetic construct
  misc_feature 1..21: note = Description of sequence: Primer. Direction: reverse primer, Name: Cleo4a3, Ligand: HIV AKA DCIR3
  source 1..21: mol_type = other DNA
tcactgctaggctcacctttg

SEQ ID NO: 128 - DNA, 20 bp, synthetic construct
  misc_feature 1..20: note = Description of sequence: Primer. Direction: Forward primer, Name: TIr4, Ligand: Microbial
  source 1..20: mol_type = other DNA
tgtcatcagggactttgctg

SEQ ID NO: 129 - DNA, 20 bp, synthetic construct
  misc_feature 1..20: note = Description of sequence: Primer. Direction: reverse primer, Name: TIr4, Ligand: Microbial
  source 1..20: mol_type = other DNA
ggactctgatcatggcactg

SEQ ID NO: 130 - DNA, 23 bp, synthetic construct
  misc_feature 1..23: note = Description of sequence: Primer. Direction: Forward primer, Name: Tlr13, Ligand: Bacterial 23 s rRna
  source 1..23: mol_type = other DNA
gttgtaacctggatgcctaagac

SEQ ID NO: 131 - DNA, 20 bp, synthetic construct
  misc_feature 1..20: note = Description of sequence: Primer. Direction: reverse primer, Name: Tlr13, Ligand: Bacterial 23 s rRna
  source 1..20: mol_type = other DNA
ggcctctgtcaagttggtga

SEQ ID NO: 132 - DNA, 21 bp, synthetic construct
  misc_feature 1..21: note = Description of sequence: Primer. Direction: Forward primer, Name: Clec5a, Ligand: Japanese encephalitis virus
  source 1..21: mol_type = other DNA
tcggggcttatcgtagtagtg

SEQ ID NO: 133 - DNA, 23 bp, synthetic construct
  misc_feature 1..23: note = Description of sequence: Primer. Direction: reverse primer, Name: Clec5a, Ligand: Japanese encephalitis virus
  source 1..23: mol_type = other DNA
tgtaggcatggtactttcgtcat

SEQ ID NO: 134 - DNA, 22 bp, synthetic construct
  misc_feature 1..22: note = Description of sequence: Primer. Direction: Forward primer, Name: Havcr2, Ligand: Unknown
  source 1..22: mol_type = other DNA
tcaggtcttaccctcaactgtg

SEQ ID NO: 135 - DNA, 23 bp, synthetic construct
  misc_feature 1..23: note = Description of sequence: Primer. Direction: reverse primer, Name: Havcr2, Ligand: Unknown
  source 1..23: mol_type = other DNA
ggcattcttaccaacctcaaaca

SEQ ID NO: 136 - DNA, 22 bp, synthetic construct
  misc_feature 1..22: note = Description of sequence: Primer. Direction: Forward primer, Name: Clec7a, Ligand: beta glucan and yeast
  source 1..22: mol_type = other DNA
gacttcagcactcaagacatcc

SEQ ID NO: 137 - DNA, 21 bp, synthetic construct
  misc_feature 1..21: note = Description of sequence: Primer. Direction: reverse primer, Name: Clec7a, Ligand: beta glucan and yeast
  source 1..21: mol_type = other DNA
ttgtgtcgccaaaatgctagg

SEQ ID NO: 138 - DNA, 21 bp, synthetic construct
  misc_feature 1..21: note = Description of sequence: Primer. Direction: Forward primer, Name: Cxcl16, Ligand: Bacteria, OxLDL, MTB
  source 1..21: mol_type = other DNA
ccttgtctcttgcgttcttcc

SEQ ID NO: 139 - DNA, 21 bp, synthetic construct
  misc_feature 1..21: note = Description of sequence: Primer. Direction: reverse primer, Name: Cxcl16, Ligand: Bacteria, OxLDL, MTB
  source 1..21: mol_type = other DNA
tccaaagtaccctgcggtatc

SEQ ID NO: 140 - DNA, 23 bp, synthetic construct
  misc_feature 1..23: note = Description of sequence: Primer. Direction: Forward primer, Name: Cd48, Ligand: MTB, E-coli
  source 1..23: mol_type = other DNA
cccaagccttccatagaaatcaa

SEQ ID NO: 141 - DNA, 23 bp, synthetic construct
  misc_feature 1..23: note = Description of sequence: Primer. Direction: reverse primer, Name: Cd48, Ligand: MTB, E-coli
  source 1..23: mol_type = other DNA
ccaagtatagtcaacatgctggt

SEQ ID NO: 142 - DNA, 21 bp, synthetic construct
  misc_feature 1..21: note = Description of sequence: Primer. Direction: Forward primer, Name: Ltf, Ligand: AGEs, Bacteria
  source 1..21: mol_type = other DNA
ggagccttgaggtgtctgaga

SEQ ID NO: 143 - DNA, 22 bp, synthetic construct
  misc_feature 1..22: note = Description of sequence: Primer. Direction: reverse primer, Name: Ltf, Ligand: AGEs, Bacteria
  source 1..22: mol_type = other DNA
aggtggcactccttgtattctg

SEQ ID NO: 144 - DNA, 20 bp, synthetic construct
  misc_feature 1..20: note = Description of sequence: Primer. Direction: Forward primer, Name: Cd74, Ligand: Bacteria (H. pylori), HIV
  source 1..20: mol_type = other DNA
agtgcgacgagaacggtaac

SEQ ID NO: 145 - DNA, 19 bp, synthetic construct
  misc_feature 1..19: note = Description of sequence: Primer. Direction: reverse primer, Name: Cd74, Ligand: Bacteria (H. pylori), HIV
  source 1..19: mol_type = other DNA
cgttggggaacacacacca

SEQ ID NO: 146 - DNA, 20 bp, synthetic construct
  misc_feature 1..20: note = Description of sequence: Primer. Direction: Forward primer, Name: Upk1b, Ligand: Bacteria (E. coli)
  source 1..20: mol_type = other DNA
cactgttcgttgcttccagg

SEQ ID NO: 147 - DNA, 23 bp, synthetic construct
  misc_feature 1..23: note = Description of sequence: Primer. Direction: reverse primer, Name: Upk1b, Ligand: Bacteria (E. coli)
  source 1..23: mol_type = other DNA
gcttcgagaagtgggtaaagact

SEQ ID NO: 148 - DNA, 19 bp, synthetic construct
  misc_feature 1..19: note = Description of sequence: Primer. Direction: Forward primer, Name: Tlr12, Ligand: Parasites 6
  source 1..19: mol_type = other DNA
gccgccattccaagctatc

SEQ ID NO: 149 - DNA, 23 bp, synthetic construct
  misc_feature 1..23: note = Description of sequence: Primer. Direction: reverse primer, Name: Tlr12, Ligand: Parasites 6
  source 1..23: mol_type = other DNA
ctccacagtccgaggtacaactt

SEQ ID NO: 150 - DNA, 20 bp, synthetic construct
  misc_feature 1..20: note = Description of sequence: Primer. Direction: Forward primer, Name: Tlr1, Ligand: Microbial
  source 1..20: mol_type = other DNA
tcaagtgtgcagctgattgc

SEQ ID NO: 151 - DNA, 20 bp, synthetic construct
  misc_feature 1..20: note = Description of sequence: Primer. Direction: reverse primer, Name: Tlr1, Ligand: Microbial
  source 1..20: mol_type = other DNA
tagtgctgacggacacatcc

SEQ ID NO: 152 - DNA, 20 bp, synthetic construct
  misc_feature 1..20: note = Description of sequence: Primer. Direction: Forward primer, Name: Tlr6, Ligand: Microbial
  source 1..20: mol_type = other DNA
tggatgtctcacacaatcgg

SEQ ID NO: 153 - DNA, 21 bp, synthetic construct
  misc_feature 1..21: note = Description of sequence: Primer. Direction: reverse primer, Name: Tlr6, Ligand: Microbial
  source 1..21: mol_type = other DNA
gcagcttagatgcaagtgagc

SEQ ID NO: 154 - DNA, 15 bp, synthetic construct
  misc_feature 1..15: note = Description of sequence: Primer. Direction: Forward primer, Name: lfitm6, Ligand: May regulate viral entry
  source 1..15: mol_type = other DNA
ggttaagagggatcc

SEQ ID NO: 155 - DNA, 15 bp, synthetic construct
  misc_feature 1..15: note = Description of sequence: Primer. Direction: reverse primer, Name: lfitm6, Ligand: May regulate viral entry
  source 1..15: mol_type = other DNA
ctttgacagtgcatg

SEQ ID NO: 156 - DNA, 21 bp, synthetic construct
  misc_feature 1..21: note = Description of sequence: Primer. Direction: Forward primer, Name: ltgam, Ligand: Fibrinogen, amyloid beta
  source 1..21: mol_type = other DNA
atggacgctgatggcaatacc

SEQ ID NO: 157 - DNA, 19 bp, synthetic construct
  misc_feature 1..19: note = Description of sequence: Primer. Direction: reverse primer, Name: ltgam, Ligand: Fibrinogen, amyloid beta
  source 1..19: mol_type = other DNA
tccccattcacgtctccca

SEQ ID NO: 158 - DNA, 23 bp, synthetic construct
  misc_feature 1..23: note = Description of sequence: Primer. Direction: Forward primer, Name: Itgb2, Ligand: CD18 binds amyloid beta
  source 1..23: mol_type = other DNA
cactgtctcagttgtgtaccaag

SEQ ID NO: 159 - DNA, 21 bp, synthetic construct
  misc_feature 1..21: note = Description of sequence: Primer. Direction: reverse primer, Name: Itgb2, Ligand: CD18 binds amyloid beta
  source 1..21: mol_type = other DNA
gctctggtgtatcacagcgaa

SEQ ID NO: 160 - DNA, 20 bp, synthetic construct
  misc_feature 1..20: note = Description of sequence: Primer. Direction: Forward primer, Name: Itgb5, Ligand: Adenovirus, Vitronectin
  source 1..20: mol_type = other DNA
gaagtgccacctcgtgtgaa

SEQ ID NO: 161 - DNA, 20 bp, synthetic construct
  misc_feature 1..20: note = Description of sequence: Primer. Direction: reverse primer, Name: Itgb5, Ligand: Adenovirus, Vitronectin
  source 1..20: mol_type = other DNA
ggaccgtggattgccaaagt

SEQ ID NO: 162 - DNA, 21 bp, synthetic construct
  misc_feature 1..21: note = Description of sequence: Primer. Direction: Forward primer, Name: Emr1, Ligand: AKA F4/80, adhesion78
  source 1..21: mol_type = other DNA
tcctgctgtgtcgtgctgttc

SEQ ID NO: 163 - DNA, 23 bp, synthetic construct
  misc_feature 1..23: note = Description of sequence: Primer. Direction: reverse primer, Name: Emr1, Ligand: AKA F4/80, adhesion78
  source 1..23: mol_type = other DNA
gccgtctggttgtcagtcttgtc

SEQ ID NO: 164 - DNA, 19 bp, synthetic construct
  misc_feature 1..19: note = Description of sequence: Primer. Direction: Forward primer, Name: Ecscr, Ligand: Filamin
  source 1..19: mol_type = other DNA
agctgtgctgggtgatcct

SEQ ID NO: 165 - DNA, 18 bp, synthetic construct
  misc_feature 1..18: note = Description of sequence: Primer. Direction: reverse primer, Name: Ecscr, Ligand: Filamin
  source 1..18: mol_type = other DNA
ttgtgggctgggagttgt

SEQ ID NO: 166 - DNA, 22 bp, synthetic construct
  misc_feature 1..22: note = Description of sequence: Primer. Direction: Forward primer, Name: Lair1, Ligand: Collagens
  source 1..22: mol_type = other DNA
gctctgaccagacctggtaagg

SEQ ID NO: 167 - DNA, 22 bp, synthetic construct
  misc_feature 1..22: note = Description of sequence: Primer. Direction: reverse primer, Name: Lair1, Ligand: Collagens
  source 1..22: mol_type = other DNA
ccatgtgtgtctccaggtgtgc

SEQ ID NO: 168 - DNA, 20 bp, synthetic construct
  misc_feature 1..20: note = Description of sequence: Primer. Direction: Forward primer, Name: Siglech, Ligand: Sialic acid
  source 1..20: mol_type = other DNA
atttctgtgaggaaaggatc

SEQ ID NO: 169 - DNA, 20 bp, synthetic construct
  misc_feature 1..20: note = Description of sequence: Primer. Direction: reverse primer, Name: Siglech, Ligand: Sialic acid
  source 1..20: mol_type = other DNA
aattcacagaactccacagc

SEQ ID NO: 170 - DNA, 20 bp, synthetic construct
  misc_feature 1..20: note = Description of sequence: Primer. Direction: Forward primer, Name: SIco2b1, Ligand: Anions
  source 1..20: mol_type = other DNA
ttggcatcggtggtgtgccc

SEQ ID NO: 171 - DNA, 22 bp, synthetic construct
  misc_feature 1..22: note = Description of sequence: Primer. Direction: reverse primer, Name: SIco2b1, Ligand: Anions
  source 1..22: mol_type = other DNA
atgcctccttctggcatccggt

SEQ ID NO: 172 - DNA, 22 bp, synthetic construct
  misc_feature 1..22: note = Description of sequence: Primer. Direction: Forward primer, Name: Slc2a5, Ligand: Glucose AKA Glut5
  source 1..22: mol_type = other DNA
ggctcatcttccccttcattca

SEQ ID NO: 173 - DNA, 22 bp, synthetic construct
  misc_feature 1..22: note = Description of sequence: Primer. Direction: reverse primer, Name: Slc2a5, Ligand: Glucose AKA Glut5
  source 1..22: mol_type = other DNA
aatgctctgcccttggtctctg

SEQ ID NO: 174 - DNA, 22 bp, synthetic construct
  misc_feature 1..22: note = Description of sequence: Primer. Direction: Forward primer, Name: S100a8, Ligand: Tlr4
  source 1..22: mol_type = other DNA
ccgtcttcaagacatcgtttga

SEQ ID NO: 175 - DNA, 22 bp, synthetic construct
  misc_feature 1..22: note = Description of sequence: Primer. Direction: reverse primer, Name: S100a8, Ligand: Tlr4
  source 1..22: mol_type = other DNA
gtagagggcatggtgatttcct

SEQ ID NO: 176 - DNA, 23 bp, synthetic construct
  misc_feature 1..23: note = Description of sequence: Primer. Direction: Forward primer, Name: S100a9, Ligand: Tlr4
  source 1..23: mol_type = other DNA
atactctaggaaggaaggacacc

SEQ ID NO: 177 - DNA, 23 bp, synthetic construct
  misc_feature 1..23: note = Description of sequence: Primer. Direction: reverse primer, Name: S100a9, Ligand: Tlr4
  source 1..23: mol_type = other DNA
tccatgatgtcatttatgagggc

SEQ ID NO: 178 - DNA, 22 bp, synthetic construct
  misc_feature 1..22: note = Description of sequence: Primer. Direction: Forward primer, Name: Lgals9, Ligand: Urate, glycans
  source 1..22: mol_type = other DNA
ctggaatccctcctgtggtgta

SEQ ID NO: 179 - DNA, 22 bp, synthetic construct
  misc_feature 1..22: note = Description of sequence: Primer. Direction: reverse primer, Name: Lgals9, Ligand: Urate, glycans
  source 1..22: mol_type = other DNA
cctcgtagcatctggcaagaca

SEQ ID NO: 180 - DNA, 23 bp, synthetic construct
  misc_feature 1..23: note = Description of sequence: Primer. Direction: Forward primer, Name: Gpr183, Ligand: Oxysterols
  source 1..23: mol_type = other DNA
gtcgtgttcatcctgtgcttcac

SEQ ID NO: 181 - DNA, 22 bp, synthetic construct
  misc_feature 1..22: note = Description of sequence: Primer. Direction: reverse primer, Name: Gpr183, Ligand: Oxysterols
  source 1..22: mol_type = other DNA
tcatcaggcacaccgtgaagtg

SEQ ID NO: 182 - DNA, 23 bp, synthetic construct
  misc_feature 1..23: note = Description of sequence: Primer. Direction: Forward primer, Name: Tmem37, Ligand: Inorganic cations?
  source 1..23: mol_type = other DNA
cagtgaaccacactgtctgagag

SEQ ID NO: 183 - DNA, 23 bp, synthetic construct
  misc_feature 1..23: note = Description of sequence: Primer. Direction: reverse primer, Name: Tmem37, Ligand: Inorganic cations?
  source 1..23: mol_type = other DNA
ggacacaatgagcatctccaagc

SEQ ID NO: 184 - DNA, 20 bp, synthetic construct
  misc_feature 1..20: note = Description of sequence: Primer. Direction: Forward primer, Name: Cd33, Ligand: Siglec 3
  source 1..20: mol_type = other DNA
gattcttgggctctgtctcg

SEQ ID NO: 185 - DNA, 20 bp, synthetic construct
  misc_feature 1..20: note = Description of sequence: Primer. Direction: reverse primer, Name: Cd33, Ligand: Siglec 3
  source 1..20: mol_type = other DNA
tggacactgctctgttcctg

SEQ ID NO: 186 - DNA, 22 bp, synthetic construct
  misc_feature 1..22: note = Description of sequence: Primer. Direction: Forward primer, Name: Gpr84, Ligand: Fatty acids
  source 1..22: mol_type = other DNA
gactgcccctcaaaagacctgc

SEQ ID NO: 187 - DNA, 20 bp, synthetic construct
  misc_feature 1..20: note = Description of sequence: Primer. Direction: reverse primer, Name: Gpr84, Ligand: Fatty acids
  source 1..20: mol_type = other DNA
gccacgccccagataattgc

SEQ ID NO: 188 - DNA, 22 bp, synthetic construct
  misc_feature 1..22: note = Description of sequence: Primer. Direction: Forward primer, Name: Slc7a7, Ligand: Amino acids
  source 1..22: mol_type = other DNA
aaggtgttggcgctgattgcag

SEQ ID NO: 189 - DNA, 22 bp, synthetic construct
  misc_feature 1..22: note = Description of sequence: Primer. Direction: reverse primer, Name: Slc7a7, Ligand: Amino acids
  source 1..22: mol_type = other DNA
agagtgccagagcaatgtcacc

SEQ ID NO: 190 - DNA, 22 bp, synthetic construct
  misc_feature 1..22: note = Description of sequence: Primer. Direction: Forward primer, Name: Cd52, Ligand: C1q
  source 1..22: mol_type = other DNA
atccttgggacaagccactacg

SEQ ID NO: 191 - DNA, 22 bp, synthetic construct
  misc_feature 1..22: note = Description of sequence: Primer. Direction: reverse primer, Name: Cd52, Ligand: C1q
  source 1..22: mol_type = other DNA
cagctgaggtagaagaggcaca

SEQ ID NO: 192 - DNA, 20 bp, synthetic construct
  misc_feature 1..20: note = Description of sequence: Primer. Direction: Forward primer, Name: Siglec5, Ligand: Sialic acid
  source 1..20: mol_type = other DNA
agcccttccagctctctacc

SEQ ID NO: 193 - DNA, 20 bp, synthetic construct
  misc_feature 1..20: note = Description of sequence: Primer. Direction: reverse primer, Name: Siglec5, Ligand: Sialic acid
  source 1..20: mol_type = other DNA
gtcccagcagctgtagaagg

SEQ ID NO: 194 - DNA, 22 bp, synthetic construct
  misc_feature 1..22: note = Description of sequence: Primer. Direction: Forward primer, Name: Cd79b, Ligand: BCR complex
  source 1..22: mol_type = other DNA
ccagcaatgacaagcagtgacc

SEQ ID NO: 195 - DNA, 23 bp, synthetic construct
  misc_feature 1..23: note = Description of sequence: Primer. Direction: reverse primer, Name: Cd79b, Ligand: BCR complex
  source 1..23: mol_type = other DNA
cctgagtggtttgtgtagcagtg

SEQ ID NO: 196 - DNA, 22 bp, synthetic construct
  misc_feature 1..22: note = Description of sequence: Primer. Direction: Forward primer, Name: Slc16a3, Ligand: Monocarboxylate
  source 1..22: mol_type = other DNA
tccatcctgctggctatgctct

SEQ ID NO: 197 - DNA, 22 bp, synthetic construct
  misc_feature 1..22: note = Description of sequence: Primer. Direction: reverse primer, Name: Slc16a3, Ligand: Monocarboxylate
  source 1..22: mol_type = other DNA
cagaaggacgcagccaccattc

SEQ ID NO: 198 - DNA, 20 bp, synthetic construct
  misc_feature 1..20: note = Description of sequence: Primer. Direction: Forward primer, Name: Icaml, Ligand: LFA-1, rhinoviruses
  source 1..20: mol_type = other DNA
caatttctcatgccgcacag

SEQ ID NO: 199 - DNA, 21 bp, synthetic construct
  misc_feature 1..21: note = Description of sequence: Primer. Direction: reverse primer, Name: Icaml, Ligand: LFA-1, rhinoviruses
  source 1..21: mol_type = other DNA
agctggaagatcgaaagtccg

SEQ ID NO: 200 - DNA, 19 bp, synthetic construct
  misc_feature 1..19: note = Description of sequence: Primer. Direction: Forward primer, Name: Icam4, Ligand: beta2 Integrins
  source 1..19: mol_type = other DNA
ggccacaagtacactctgc

SEQ ID NO: 201 - DNA, 21 bp, synthetic construct
  misc_feature 1..21: note = Description of sequence: Primer. Direction: reverse primer, Name: Icam4, Ligand: beta2 Integrins
  source 1..21: mol_type = other DNA
ggcttaaagcgaggactgtca

SEQ ID NO: 202 - DNA, 20 bp, synthetic construct
  misc_feature 1..20: note = Description of sequence: Primer. Direction: Forward primer, Name: Cd84, Ligand: CD84 (Homophilic)
  source 1..20: mol_type = other DNA
ttgttccgtttgttcaagag

SEQ ID NO: 203 - DNA, 21 bp, synthetic construct
  misc_feature 1..21: note = Description of sequence: Primer. Direction: reverse primer, Name: Cd84, Ligand: CD84 (Homophilic)
  source 1..21: mol_type = other DNA
cggaataaactgtgttcactg

SEQ ID NO: 204 - DNA, 20 bp, synthetic construct
  misc_feature 1..20: note = Description of sequence: Primer. Direction: Forward primer, Name: Lag-3, Ligand: Binds MHC II
  source 1..20: mol_type = other DNA
ccaggcctcgatgattgcta

SEQ ID NO: 205 - DNA, 20 bp, synthetic construct
  misc_feature 1..20: note = Description of sequence: Primer. Direction: reverse primer, Name: Lag-3, Ligand: Binds MHC II
  source 1..20: mol_type = other DNA
cagcagcgtacactgtcaga

SEQ ID NO: 206 - DNA, 20 bp, synthetic construct
  misc_feature 1..20: note = Description of sequence: Primer. Direction: Forward primer, Name: Cd86, Ligand: CD28 on T cells
  source 1..20: mol_type = other DNA
catgggcttggcaatcctta

SEQ ID NO: 207 - DNA, 20 bp, synthetic construct
  misc_feature 1..20: note = Description of sequence: Primer. Direction: reverse primer, Name: Cd86, Ligand: CD28 on T cells
  source 1..20: mol_type = other DNA
aaatgggcacggcagatatg

SEQ ID NO: 208 - DNA, 20 bp, synthetic construct
  misc_feature 1..20: note = Description of sequence: Primer. Direction: Forward primer, Name: Ptprc, Ligand: Galectin-1
  source 1..20: mol_type = other DNA
gggttgttctgtgccttgtt

SEQ ID NO: 209 - DNA, 20 bp, synthetic construct
  misc_feature 1..20: note = Description of sequence: Primer. Direction: reverse primer, Name: Ptprc, Ligand: Galectin-1
  source 1..20: mol_type = other DNA
ctggacggacacagttagca

SEQ ID NO: 210 - DNA, 23 bp, synthetic construct
  misc_feature 1..23: note = Description of sequence: Primer. Direction: Forward primer, Name: Dap12, Ligand: Lipids
  source 1..23: mol_type = other DNA
ccagcccctggactgtggtgtcc

SEQ ID NO: 211 - DNA, 23 bp, synthetic construct
  misc_feature 1..23: note = Description of sequence: Primer. Direction: reverse primer, Name: Dap12, Ligand: Lipids
  source 1..23: mol_type = other DNA
gtaccctgtggatctgtattcca

SEQ ID NO: 212 - DNA, 22 bp, synthetic construct
  misc_feature 1..22: note = Description of sequence: Primer. Direction: Forward primer, Name: Tnfrsf13b, Ligand: BLyS
  source 1..22: mol_type = other DNA
agcgcacctgtgcagccttctg

SEQ ID NO: 213 - DNA, 21 bp, synthetic construct
  misc_feature 1..21: note = Description of sequence: Primer. Direction: reverse primer, Name: Tnfrsf13b, Ligand: BLyS
  source 1..21: mol_type = other DNA
gccccgggagagctggacttg

SEQ ID NO: 214 - DNA, 24 bp, synthetic construct
  misc_feature 1..24: note = Description of sequence: Primer. Direction: Forward primer, Name: Tnfrsf17, Ligand: BAFF
  source 1..24: mol_type = other DNA
ctaaggaagataaactctgaacca

SEQ ID NO: 215 - DNA, 24 bp, synthetic construct
  misc_feature 1..24: note = Description of sequence: Primer. Direction: reverse primer, Name: Tnfrsf17, Ligand: BAFF
  source 1..24: mol_type = other DNA
ttacctagcagaaattgatttctc

SEQ ID NO: 216 - DNA, 21 bp, synthetic construct
  misc_feature 1..21: note = Description of sequence: Primer. Direction: Forward primer, Name: Cd22, Ligand: CD45, IgM
  source 1..21: mol_type = other DNA
ccactcctcaggccagaaact

SEQ ID NO: 217 - DNA, 20 bp, synthetic construct
  misc_feature 1..20: note = Description of sequence: Primer. Direction: reverse primer, Name: Cd22, Ligand: CD45, IgM
  source 1..20: mol_type = other DNA
tgccgatggtctctggactg

SEQ ID NO: 218 - DNA, 20 bp, synthetic construct
  misc_feature 1..20: note = Description of sequence: Primer. Direction: Forward primer, Name: Tmem119, Ligand: AKA OBIF
  source 1..20: mol_type = other DNA
gtgtctaacaggccccagaa

SEQ ID NO: 219 - DNA, 20 bp, synthetic construct
  misc_feature 1..20: note = Description of sequence: Primer. Direction: reverse primer, Name: Tmem119, Ligand: AKA OBIF
  source 1..20: mol_type = other DNA
agccacgtggtatcaaggag

SEQ ID NO: 220 - DNA, 20 bp, synthetic construct
  misc_feature 1..20: note = Description of sequence: Primer. Direction: Forward primer, Name: Cd53, Ligand: tetraspanin
  source 1..20: mol_type = other DNA
tcctccttgctgaggtgacc

SEQ ID NO: 221 - DNA, 20 bp, synthetic construct
  misc_feature 1..20: note = Description of sequence: Primer. Direction: reverse primer, Name: Cd53, Ligand: tetraspanin
  source 1..20: mol_type = other DNA
agggtcagtgcaaaggacat

SEQ ID NO: 222 - DNA, 20 bp, synthetic construct
  misc_feature 1..20: note = Description of sequence: Primer. Direction: Forward primer, Name: Slamf9, Ligand: Unknown
  source 1..20: mol_type = other DNA
cagggctttacaacgcccaa

SEQ ID NO: 223 - DNA, 23 bp, synthetic construct
  misc_feature 1..23: note = Description of sequence: Primer. Direction: reverse primer, Name: Slamf9, Ligand: Unknown
  source 1..23: mol_type = other DNA
gtagacacggagatggtaagact

SEQ ID NO: 224 - DNA, 21 bp, synthetic construct
  misc_feature 1..21: note = Description of sequence: Primer. Direction: Forward primer, Name: Cleo4b1, Ligand: Unknown
  source 1..21: mol_type = other DNA
ccactgctacttggttcccac

SEQ ID NO: 225 - DNA, 21 bp, synthetic construct
  misc_feature 1..21: note = Description of sequence: Primer. Direction: reverse primer, Name: Cleo4b1, Ligand: Unknown
  source 1..21: mol_type = other DNA
tcctcctggctatggatcacc

SEQ ID NO: 226 - DNA, 19 bp, synthetic construct
  misc_feature 1..19: note = Description of sequence: Primer. Direction: Forward primer, Name: Lilra5, Ligand: Unknown
  source 1..19: mol_type = other DNA
cggaagggaatccgcacaa

SEQ ID NO: 227 - DNA, 21 bp, synthetic construct
  misc_feature 1..21: note = Description of sequence: Primer. Direction: reverse primer, Name: Lilra5, Ligand: Unknown
  source 1..21: mol_type = other DNA
cacctcacatgagatggtcac

SEQ ID NO: 228 - DNA, 20 bp, synthetic construct
  misc_feature 1..20: note = Description of sequence: Primer. Direction: Forward primer, Name: mGapdh, Ligand: Control
  source 1..20: mol_type = other DNA
ggtcggtgtgaacggatttg

SEQ ID NO: 229 - DNA, 22 bp, synthetic construct
  misc_feature 1..22: note = Description of sequence: Primer. Direction: reverse primer, Name: mGapdh, Ligand: Control
  source 1..22: mol_type = other DNA
ggagtcatactggaacatgtag

SEQ ID NO: 230 - DNA, 20 bp, synthetic construct
  misc_feature 1..20: note = Description of sequence: Primer. Direction: Forward primer, Name: mActb, Ligand: Control
  source 1..20: mol_type = other DNA
ctaaggccaaccgtgaaaag

SEQ ID NO: 231 - DNA, 20 bp, synthetic construct
  misc_feature 1..20: note = Description of sequence: Primer. Direction: reverse primer, Name: mActb, Ligand: Control
  source 1..20: mol_type = other DNA
accagaggcatacagggaca

SEQ ID NO: 232 - DNA, 21 bp, synthetic construct
  misc_feature 1..21: note = Description of sequence: Primer. Direction: Forward primer, Name: mRn18s, Ligand: Control
  source 1..21: mol_type = other DNA
gatccattggagggcaagtct

SEQ ID NO: 233 - DNA, 24 bp, synthetic construct
  misc_feature 1..24: note = Description of sequence: Primer. Direction: reverse primer, Name: mRn18s, Ligand: Control
  source 1..24: mol_type = other DNA
ccaagatccaactacgagcttttt

SEQ ID NO: 234 - DNA, 21 bp, synthetic construct
  misc_feature 1..21: note = Description of sequence: Primer. Primer name: Tnf-alpha-1 forward primer
  source 1..21: mol_type = other DNA
cacagaaagcatgatccgcga

SEQ ID NO: 235 - DNA, 21 bp, synthetic construct
  misc_feature 1..21: note = Description of sequence: Primer. Primer name: Tnf-alpha-1 reverse primer
  source 1..21: mol_type = other DNA
cggcagagaggaggttgactt

SEQ ID NO: 236 - DNA, 20 bp, synthetic construct
  misc_feature 1..20: note = Description of sequence: Primer. Primer name: Iba1 forward primer
  source 1..20: mol_type = other DNA
gtccttgaagcgaatgctgg

SEQ ID NO: 237 - DNA, 20 bp, synthetic construct
  misc_feature 1..20: note = Description of sequence: Primer. Primer name: Iba1 reverse primer
  source 1..20: mol_type = other DNA
cattctcaagatggcagatc

SEQ ID NO: 238 - DNA, 20 bp, synthetic construct
  misc_feature 1..20: note = Description of sequence: Primer. Primer name: Gapdh forward primer
  source 1..20: mol_type = other DNA
ggtcggtgtgaacggatttg

SEQ ID NO: 239 - DNA, 22 bp, synthetic construct
  misc_feature 1..22: note = Description of sequence: Primer. Primer name: Gapdh reverse primer
  source 1..22: mol_type = other DNA
ggagtcatactggaacatgtag

SEQ ID NO: 240 - DNA, 20 bp, synthetic construct
  misc_feature 1..20: note = Description of sequence: Primer. Primer name: Ruminococcaceae forward primer
  source 1..20: mol_type = other DNA
actgagaggttgaacggcca

SEQ ID NO: 241 - DNA, 23 bp, synthetic construct
  misc_feature 1..23: note = Description of sequence: Primer. Primer name: Ruminococcaceae reverse primer
  source 1..23: mol_type = other DNA
cctttacacccagtaawtccgga

SEQ ID NO: 242 - DNA, 16 bp, synthetic construct
  misc_feature 1..16: note = Description of sequence: Primer. Primer name: Clostridiaceae forward primer
  source 1..16: mol_type = other DNA
gcacaagcagtggagt

SEQ ID NO: 243 - DNA, 18 bp, synthetic construct
  misc_feature 1..18: note = Description of sequence: Primer. Primer name: Clostridiaceae reverse primer
  source 1..18: mol_type = other DNA
cttcctccgttttgtcaa

SEQ ID NO: 244 - DNA, 19 bp, synthetic construct
  misc_feature 1..19: note = Description of sequence: Primer. Primer name: Lachnospiraceae forward primer
  source 1..19: mol_type = other DNA
cggtacctgactaagaagc

SEQ ID NO: 245 - DNA, 19 bp, synthetic construct
  misc_feature 1..19: note = Description of sequence: Primer. Primer name: Lachnospiraceae reverse primer
  source 1..19: mol_type = other DNA
agtttyattcttgcgaacg

SEQ ID NO: 246 - DNA, 19 bp, synthetic construct
  misc_feature 1..19: note = Description of sequence: Primer. Primer name: 16S Universal forward primer
  source 1..19: mol_type = other DNA
ctcctacgggaggcagcag

SEQ ID NO: 247 - DNA, 18 bp, synthetic construct
  misc_feature 1..18: note = Description of sequence: Primer. Primer name: 16S Universal reverse primer
  source 1..18: mol_type = other DNA
gtattaccgcggctgctg

SEQ ID NO: 248 - DNA, 17 bp, synthetic construct
  misc_feature 1..17: note = Description of sequence: Primer. Primer name: Brigitvirus forward primer
  source 1..17: mol_type = other DNA
ttctgccgctcgtatct

SEQ ID NO: 249 - DNA, 20 bp, synthetic construct
  misc_feature 1..20: note = Description of sequence: Primer. Primer name: Brigitvirus reverse primer
  source 1..20: mol_type = other DNA
gagctgcatcttctaccttg

SEQ ID NO: 250 - DNA, 20 bp, synthetic construct
  misc_feature 1..20: note = Description of sequence: Primer. Primer name: Oengusvirus forward primer
  source 1..20: mol_type = other DNA
gtgagctacgagacctatca

SEQ ID NO: 251 - DNA, 20 bp, synthetic construct
  misc_feature 1..20: note = Description of sequence: Primer. Primer name: Oengusvirus reverse primer
  source 1..20: mol_type = other DNA
ggcaaagggtctcttcatac

SEQ ID NO: 252 - DNA, 20 bp, synthetic construct
  misc_feature 1..20: note = Description of sequence: Primer. Primer name: Lugh forward primer
  source 1..20: mol_type = other DNA
ccgttacctgagtgacaaag

SEQ ID NO: 253 - DNA, 18 bp, synthetic construct
  misc_feature 1..18: note = Description of sequence: Primer. Primer name: Lugh reverse primer
  source 1..18: mol_type = other DNA
ctgccttactgcgtttca

SEQ ID NO: 254 - DNA, 20 bp, synthetic construct
  misc_feature 1..20: note = Description of sequence: Primer. Primer name: Siphov-Ring forward primer
  source 1..20: mol_type = other DNA
gaggaccgtgtaaaggaatg

SEQ ID NO: 255 - DNA, 20 bp, synthetic construct
  misc_feature 1..20: note = Description of sequence: Primer. Primer name: Siphov-Ring reverse primer
  source 1..20: mol_type = other DNA
cgagcactacctgactaaac

SEQ ID NO: 256 - DNA, 19 bp, synthetic construct
  misc_feature 1..19: note = Description of sequence: Primer. Primer name: Siphov-Fury forward primer
  source 1..19: mol_type = other DNA
cccagagcaactgttgaag

SEQ ID NO: 257 - DNA, 21 bp, synthetic construct
  misc_feature 1..21: note = Description of sequence: Primer. Primer name: Siphov-Fury reverse primer
  source 1..21: mol_type = other DNA
gtgatcgaggacttcatcttg

SEQ ID NO: 258 - DNA, 22 bp, synthetic construct
  misc_feature 1..22: note = Description of sequence: Primer. Primer name: Siphov-c-st forward primer
  source 1..22: mol_type = other DNA
ccgtaggatatgctcaaactta

SEQ ID NO: 259 - DNA, 18 bp, synthetic construct
  misc_feature 1..18: note = Description of sequence: Primer. Primer name: Siphov-c-st reverse primer
  source 1..18: mol_type = other DNA
gttcctcctgccattgtt

SEQ ID NO: 260 - DNA, 20 bp, synthetic construct
  misc_feature 1..20: note = Description of sequence: Primer. Primer name: Podov-CPS1 forward primer
  source 1..20: mol_type = other DNA
ttgttacggcatatgctaga

SEQ ID NO: 261 - DNA, 21 bp, synthetic construct
  misc_feature 1..21: note = Description of sequence: Primer. Primer name: Podov-CPS1 reverse primer
  source 1..21: mol_type = other DNA
gctgtcagtatcgctgtataa

SEQ ID NO: 262 - DNA, 18 bp, synthetic construct
  misc_feature 1..18: note = Description of sequence: Primer. Primer name: Podov-CPS2 forward primer
  source 1..18: mol_type = other DNA
gggtgggacgttgaattt

SEQ ID NO: 263 - DNA, 20 bp, synthetic construct
  misc_feature 1..20: note = Description of sequence: Primer. Primer name: Podov-CPS2 reverse primer
  source 1..20: mol_type = other DNA
tccgttactgtctgataggg

SEQ ID NO: 264 - DNA, 20 bp, synthetic construct
  misc_feature 1..20: note = Description of sequence: Primer. Primer name: Siphov-phi3626 forward primer
  source 1..20: mol_type = other DNA
gcaggagcatcagttgataa

SEQ ID NO: 265 - DNA, 20 bp, synthetic construct
  misc_feature 1..20: note = Description of sequence: Primer. Primer name: Siphov-phi3626 reverse primer
  source 1..20: mol_type = other DNA
gcaagtagcccatagtcatc

SEQ ID NO: 266 - DNA, 20 bp, synthetic construct
  misc_feature 1..20: note = Description of sequence: Primer. Primer name: Myo-phiCDHM19 forward primer
  source 1..20: mol_type = other DNA
tcctccgatacatccaagat

SEQ ID NO: 267 - DNA, 18 bp, synthetic construct
  misc_feature 1..18: note = Description of sequence: Primer. Primer name: Myo-phiCDHM19 reverse primer
  source 1..18: mol_type = other DNA
gtcgtgctgttctcgttt

SEQ ID NO: 268 - DNA, 20 bp, synthetic construct
  misc_feature 1..20: note = Description of sequence: Primer. Primer name: Myo-phiCT19406B forward primer
  source 1..20: mol_type = other DNA
gcaaagagctcctcaataca

SEQ ID NO: 269 - DNA, 20 bp, synthetic construct
  misc_feature 1..20: note = Description of sequence: Primer. Primer name: Myo-phiCT19406B reverse primer
  source 1..20: mol_type = other DNA
cactcatttggtcgcatttc

SEQ ID NO: 270 - DNA, 19 bp, synthetic construct
  misc_feature 1..19: note = Description of sequence: Primer. Primer name: peu-miR2916 forward primer
  source 1..19: mol_type = other DNA
gccgaccagggatcggtgg

SEQ ID NO: 271 - DNA, 22 bp, synthetic construct
  misc_feature 1..22: note = Description of sequence: Primer. Primer name: RNU6 forward primer
  source 1..22: mol_type = other DNA
cgcaaggatgacacgcaaattc

SEQ ID NO: 272 - DNA, 43 bp, synthetic construct
  misc_feature 1..43: note = Description of sequence: Primer. Primer name: S1008A-promoter forward primer
  source 1..43: mol_type = other DNA
tagcccatatatggagttccaagcttttgtaagataatcgtgg

SEQ ID NO: 273 - DNA, 42 bp, synthetic construct
  misc_feature 1..42: note = Description of sequence: Primer. Primer name: S1008A-promoter reverse primer
  source 1..42: mol_type = other DNA
atctgacggttcactaaaccatgtgaggctaagtgtcagctg

SEQ ID NO: 274 - DNA, 21 bp, synthetic construct
  misc_feature 1..21: note = Description of sequence: Primer. Primer name: pLuc163523 forward primer
  source 1..21: mol_type = other DNA
ggtttagtgaaccgtcagatc

SEQ ID NO: 275 - DNA, 22 bp, synthetic construct
  misc_feature 1..22: note = Description of sequence: Primer. Primer name: pLuc163523 reverse primer
  source 1..22: mol_type = other DNA
ggaactccatatatgggctatg

SEQ ID NO: 276 - DNA, 26 bp, synthetic construct
  misc_feature 1..26: note = Description of sequence: Primer. Primer name: S1008A-prom-Mut forward primer
  source 1..26: mol_type = other DNA
aaggtaaagctttcctctataaaagc

SEQ ID NO: 277 - DNA, 20 bp, synthetic construct
  misc_feature 1..20: note = Description of sequence: Primer. Primer name: S1008A-prom-Mut reverse primer
  source 1..20: mol_type = other DNA
ctgcccacagcctcgggcta

SEQ ID NO: 278 - DNA, 14 bp, synthetic construct
  misc_feature 1..14: note = Description of sequence: IAA binding motif
  source 1..14: mol_type = other DNA
  misc_feature 4: note = n is a, c, g, or t
  misc_feature 11: note = n is a, c, g, or t
tggncagctgncca

SEQ ID NO: 279 - DNA, 45 bp, Mus musculus
  misc_feature 1..45: note = Description of sequence: S100A8 ortholog
  source 1..45: mol_type = genomic DNA
tagcccacctgtggggctcgccaactttcctctataaaagcagcc

SEQ ID NO: 280 - DNA, 45 bp, Homo sapiens
  misc_feature 1..45: note = Description of sequence: S100A8 ortholog
  source 1..45: mol_type = genomic DNA
tgcctgaagcgtggggctcgccaacctaaccctataaaaagagcc

SEQ ID NO: 281 - DNA, 43 bp, Gorilla gorilla
  misc_feature 1..43: note = Description of sequence: S100A8 ortholog
  source 1..43: mol_type = genomic DNA
tagccgagcctggggctggccaacttcctctataaaaagagcc

SEQ ID NO: 282 - DNA, 42 bp, Bos taurus
  misc_feature 1..42: note = Description of sequence: S100A8 ortholog
  source 1..42: mol_type = genomic DNA
tgccaggcccggggctggccaatctggcttataaaaagagcc

SEQ ID NO: 283 - DNA, 46 bp, Equus ferus
  misc_feature 1..46: note = Description of sequence: S100A8 ortholog
  source 1..46: mol_type = genomic DNA
tgtctgaggcgccggggctggccaacagctctataaaaacgagcca

SEQ ID NO: 284 - DNA, 46 bp, Orycteropus afer
  misc_feature 1..46: note = Description of sequence: S100A8 ortholog
  source 1..46: mol_type = genomic DNA
tcccaaggctgggtcgtgttggcctgcactaactctataaaggagc

SEQ ID NO: 285 - DNA, 21 bp, synthetic construct
  misc_feature 1..21: note = Description of sequence: IAA binding oligo from Figure 4C
  source 1..21: mol_type = other DNA
gctgtgggcagctggccaagc

SEQ ID NO: 286 - DNA, 21 bp, synthetic construct
  misc_feature 1..21: note = Description of sequence: IAA binding oligo from Figure 4C
  source 1..21: mol_type = other DNA
gctgtatgcasttggccaagc

SEQ ID NO: 287 - DNA, 18 bp, synthetic construct
  misc_feature 1..18: note = Description of sequence: Hairpin structure fom Fugure 5A-5C
  source 1..18: mol_type = other DNA
gtgggcagctggccaagc

(19) United States
(12) Patent Application Publication
(10) Pub. No.: US 2025/0136997 A1
(43) Pub. Date: May 1, 2025

(54) COMPOSITIONS FOR SEQUESTERING ISOAMYL AMINE AND METHODS OF USE THEREFOR

(71) Applicant: University of Louisville Research Foundation, Inc., Louisville, KY (US)

(72) Inventor: Huang-Ge Zhang, Louisville, KY (US)

(21) Appl. No.: 18/680,457

(22) Filed: May 31, 2024

Related U.S. Application Data
(60) Provisional application No. 63/505,372, filed on May 31, 2023.

(51) Int. Cl. C12N 15/115; A61P 25/28; C07H 21/00; C12N 15/10

(52) U.S. Cl. CPC C12N 15/115; A61P 25/28; C07H 21/00; C12N 15/1065

(57) ABSTRACT

Provided are compositions that include an isoamyl alcohol (IAA) sequestration agent, wherein the IAA sequestration agent includes an IAA binding moiety. In some embodiments, the IAA binding moiety is a nucleotide sequence or a peptide or polypeptide (e.g., and antibody or a fragment thereof that includes a paratope) to which IAA binds. In some embodiments, the composition further includes comprising a pharmaceutically acceptable carrier, excipient, or diluent, optionally a pharmaceutically acceptable carrier, excipient, or diluent that is pharmaceutically acceptable for use in a human. The presently disclosed subject matter also provides oligonucleotides, optionally further including a tag, and/or the oligonucleotide includes at least one modified base. Also provided are methods for using the same to reduce and/or delay cognitive decline associated with neurodegenerative disease and/or for sequestering isoamyl alcohol (IAA) produced by microbiota in the gut of subjects.

### CROSS REFERENCE TO RELATED APPLICATIONS

This application claims the benefit of U.S. Provisional Application No. 63/505,372, filed on May 31, 2023, which is incorporated herein by reference in its entirety.

### STATEMENT OF GOVERNMENT SUPPORT

This invention was made with government support under grant number AT008617 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Sep. 16, 2024, is entitled “11258-037US1_ST26.xml”, and is 381,364 bytes in size.

### BACKGROUND

Metabolism is a key area where the host and microbiota interact. Recent advances have clearly defined a role for gut microbiome metabolites in disease initiation and progression. Bacterial metabolites appear to have the potential to alter physiological pathways of host cells and in specific circumstances, such as in aging and cancer, to modulate the expression of genes and switch the function of a gene, such as p53, therefore affecting the disease process. However, whether bacterial metabolites can affect the expression of genes by directly modulating transcription machinery is not known.

Gut microbiota and the brain communicate with each other via various routes, involving gut microbial metabolites. The composition of altered aged gut microbiota accelerates the aging process by way of toxic gut metabolites. As key players in the brain's immune function, microglia shape neuronal wiring and activity, synaptic plasticity, and phagocytosis and support the survival of neurons and neuronal progenitors via the secretion of growth factors. During the aging process, microglia develop into a highly reactive and unbalanced state promoting cognitive dysfunction including altered brain plasticity and neurodegeneration.

Sensing changes in the brain milieu is a major microglial function that regulates the ability of these cells to perform other tasks including host defense and homeostasis. Microglia express a cluster of genes including S100A8 and S100A9 that allow them to perform their sensing functions termed the sensome. The increasing release of the heterodimer formed by S100A8 and S100A9 proteins is associated with aging. This heterodimer is a toll-like receptor 4 (TLR4) and receptor for advanced glycation end products (RAGE) ligand that is situated upstream of tumor necrosis factor alpha (TNF-α), CXCL synthesis and secretion, promotes NF-kB activation, and secretion of multiple inflammatory proteins, such as IL-6. S100A8 and S100A9 proteins are increased with aging in the mouse brain and promote microglial cell apoptosis and inflammatory cytokine induction and play a role in local and systemic inflammation. However, whether the expression of S100A8 and S100A9 is regulated by gut microbiome metabolite-mediated transcription machinery is not known.

Gene expression is regulated by the binding of transcription factors (TFs) to specific DNA sequence motifs across the genome. TFs do not always have access to these binding sites depending on the availability of these sites that are exposed to TFs. Because there is no available approach to label metabolites to demonstrate the direct interaction between the promoter region and metabolites, there is an inability to know whether gut microbiome metabolites regulate the accessibility of TFs to its regulated genes via direct binding to its motif region. Developing such technology is significant since unaccountable and numerous small molecules released from gut microbiomes regulate the expression of genes in host cells and play a physiological and pathophysiological role.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In one aspect, disclosed herein are methods of detecting the binding site of a small molecule to a target DNA sequence, the method comprising a) obtaining a short single-strand DNA oligonucleotide of a target DNA sequence; b) incubate DNA oligonucleotide with the small molecule creating a sample mixture; and c) performing nondenaturing polyacrylamide gel electrophoresis (PAGE) on the sample mixture, whereby a band on the gel is revealed for the sample mixture; wherein a shift in the band for the sample mixture relative to a control containing only the DNA oligonucleotide indicates the binding site for the small molecule to the target DNA sequence is present in the DNA oligonucleotide.

Also disclosed herein are methods of screening for a small molecule that binds to a target DNA sequence, the method comprising a) obtaining a short single-strand DNA oligonucleotide of a target DNA sequence; b) incubate DNA oligonucleotide with the small molecule creating a sample mixture; and c) performing nondenaturing polyacrylamide gel electrophoresis (PAGE) on the sample mixture, whereby a band on the gel is revealed for the sample mixture; wherein a shift in the band for the sample mixture relative to a control containing only the DNA oligonucleotide indicates the small molecule binds the target DNA sequence.

In some embodiments, the presently disclosed subject matter relates to compositions that include an IAA sequestration agent and methods for using the same to inhibit or treat diseases, disorders, and conditions associated with undesirable IAA biological activities.

In some embodiments, the IAA sequestration agent comprises an oligonucleotide to which IAA can bind. In some embodiments, an oligonucleotide of the presently disclosed subject matter comprises, consists essentially of, or consists of the sequence 5′-TGGGCAGCTGGCCA-3′ (SEQ ID NO: 10), optionally comprising, consisting essentially of, or consisting of the sequence 5′-CTGTGGGCAGCTGGCCAAGC-3′ (SEQ ID NO: 11). In some embodiments, the oligonucleotide further comprises a tag, optionally a biotin tag.

In some embodiments, the presently disclosed subject matter also relates to methods for reducing and/or delaying cognitive decline associated with neurodegenerative disease in subjects in need thereof. In some embodiments, the methods comprise, consist essentially of, or consist of administering to a subject in need thereof an effective amount of a composition comprising, consisting essentially of, or consisting of an IAA sequestration agent of the presently disclosed subject matter. In some embodiments, the IAA sequestration agent is an oligonucleotide that comprises an IAA binding sequence as disclosed herein.

In some embodiments, the presently disclosed subject matter also relates to methods for sequestering isoamyl alcohol (IAA) produced by microbiota in the gut of a subject. In some embodiments, the method comprising administering to the subject an effective amount of a composition comprising, consisting essentially of, or consisting of an IAA sequestration agent of the presently disclosed subject matter. In some embodiments, the IAA sequestration agent is an oligonucleotide that comprises an IAA binding sequence as disclosed herein.

In some embodiments of the presently disclosed methods, the composition is administered orally.

In some embodiments of the presently disclosed methods, the composition is administered intravenously.

In some embodiments of the presently disclosed methods, the composition is administered topically.

In some embodiments of the presently disclosed methods, the subject is a mammal. In some embodiments, the subject is a mouse. In some embodiments, the subject is a human.

Thus, it is an object of the presently disclosed subject matter to provide compositions and methods for inhibiting microglial S100A8 signaling, which in some embodiments can reduce, delay, or eliminate cognitive decline associated with microglial S100A8 signaling in neurodegenerative conditions.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1F. Comparative analysis of sensome genes expression and apoptosis in microglia of young and aged mice. (FIG. 1A) Microglial cells isolated from mouse brains by Percoll gradient centrifugation and sorted by FACS with gating on the CD11b+CD45med population (red circle). (FIG. 1B) A heat map showing aging genes in microglia significantly (p<0.05) changed between 2-month-old (young, Y) mice and 12-month-old (aged, A) mice (n=3, each group mixed from three mice) using a quantitative real-time (qPCR) array. Red and blue colors represent elevated and decreased expression of mRNAs, respectively. (FIG. 1C) An individual qPCR analysis of microglia recovered from FACS sorted CD11b+CD45med cells from different regions of mouse brain specimens. The ratio of each gene to that of GAPDH was calculated, and the relative expression levels normalized by cerebrum in the young mouse PBS group are shown. Red and blue colors represent elevated and decreased expression of mRNAs, respectively. (FIG. 1D) Western blot analysis of microglial cells from cerebral cortex. GAPDH was used as a loading control. The numbers under the blots indicate the density of bands normalized to the density of GAPDH. (FIG. 1E) Analysis of apoptosis in the brain of mice using the TUNEL assay. Scale bars, 100 mm. (FIG. 1F) Microglial cells isolated from mice and transfected with S100A8, S100A9 overexpression (pS100A8 and pS100A9), and knockout (KO) plasmids, respectively. Western blot analysis of the expression of S100A8, S100A9, and cleaved caspase-3 in microglia. Data are representative of three independent experiments.

FIGS. 2A-2F. Gut bacterial metabolites IAA and CA promote apoptosis of microglia cells in aged mice. (FIG. 2A) Analysis workflow of the relationship between gut microbe metabolites and brain microglial cell sensome. (FIG. 2B) Metabolite profiles of gut feces from 2- and 12-month-old-specific pathogen-free (SPF) and germ-free (GF) mice (n=2, each group mixed from three mice) using liquid chromatography-mass spectrometry (LC-MS). The color scale represents log 10 of signal intensity of MS data. (FIG. 2C) Integrated data of metabolite profiles and expression of sensome genes from young and aged mice (n=6), linear regression analysis indicating the relationship between the gut metabolites isoamylamine (IAA), crotonic acid (CA), and the S100A8 of microglia. (FIG. 2D) Quantification of IAA and CA in gut feces using high-performance liquid chromatography (HPLC). (FIG. 2E) Western blot analysis of cleaved caspase-3 and PARP in microglia of mice. GAPDH was used as a loading control. The numbers under the blots indicate the density of bands normalized to the density of GAPDH. (FIG. 2F) Analysis of apoptosis bodies (ABs) in the medium of primary microglia cells from mouse with FACS (left). Quantification of ABs (right). Data are representative of three independent experiments (error bars, SD). *p<0.05, **p<0.01 (two-tailed t test).

FIGS. 3A-3H. Aging-dependent reduction of gut bacteriophages leads to the production of bacterial IAA and CA. (FIG. 3A) A heat map showing the composition of bacteria (family level) in the feces from healthy young (n=12) and aged people (n=12) using 16S rRNA next generation sequencing (NGS) analysis. (FIG. 3B) Principal coordinates analysis (PCoA) of 16S rRNA sequencing. (FIG. 3C) Real-time qPCR analysis of selected bacteria in mice fecal samples (n=10). Fold changes are shown relative to young mice. (FIG. 3D) Predictive analysis of the potential of bacteriophages (family level) to target higher abundance bacteria Ruminococcaceae and Clostridiaceae in the aged mouse intestine. (FIG. 3E) Analysis of phage level in feces of mice using qPCR. (FIG. 3F) Schematic diagram of Ruminococcaceae (ATCC, TSD-27)-binding phages isolated from human feces following the administration to TSD-27 and mice (top). TSD-27 exposed to TSD-27 binding phages (phageTSD-27). The phage titer and relative bacterial survival rate estimated by using a spectrophotometer (bottom). (FIG. 3G) HPLC analysis of the metabolites IAA and CA in the growth medium of TSD-27 treated with phageTSD-27 (left) and in the fecal supernatants of mice gavage-given phage TSD-27 (right). (FIG. 3H) A heat map showing the relative abundance of TSD-27 binding phage and Lactobacillus rhamnosus GG (LGG) binding phage in human feces, as well as phage from young and old subjects using NGS analysis. Data are representative of four independent experiments (error bars, SD). *p<0.05, **p<0.01 (two-tailed t test); NS, not significant.

FIGS. 4A-4F. Identification of gut metabolite IAA-targeted S100A8 promoter region. (FIG. 4A) Schematic diagram of the strategy to demonstrate the interaction of IAA and the promoter of S100A8 (left). Oligo binding to IAA with the expected mobility shift on PAGE (right). The transcription start site (TSS) is marked by the bent arrow. ATG; translation start code. (FIG. 4B) 10 pmol of synthetic DNA oligo S100p1 and S100p2 (60 mer/each) corresponding to the sequence on the promoter of S100A8 incubated with IAA (1 mM) or fecal supernatant (1 g feces/mL) for 30 min at 37-C. The oligos separated on 15% PAGE and visualized with ethidium bromide. P, PBS; I, IAA; S, gut feces supernatant. (FIG. 4C) The shorter synthetic DNA oligos S100p1-A to S100p1-I (20 mer/each) correspond to the sequence of S100p1 (top). Representative PAGE for the oligos S10p1-F to S100p1-I with or without IAA (bottom) SEQ ID NOs: 285 and 286, respectively top to bottom)(FIG. 4D) SPR analysis of the interaction of biotin-labeled oligos S100p1, S100p1-G, and mutant S100p1-GM with IAA (1 mM). (FIG. 4E) The promoter sequences of S100A8 inserted into a luciferase reporter pLuc and transfection of microglia. Luciferase activities assessment 12 h after treatment of IAA. (FIG. 4F) Representation of a 15% PAGE for the oligo S1001-G, as well as mutants indicated in the figure. Each oligo contained a single base mutation. The base in pink replaced by different base caused the abolishment of IAA binding shift. Data are representative of three independent experiments (error bars, SD). *p<0.05, **p<0.01 (two-tailed t test); NS, not significant.

FIGS. 5A-5J. IAA, a promoter-unfolding enabler, facilitates p53 access to the S100A8 promoter region via unwinding of the hairpin structure (SEQ ID NO: 287). (FIG. 5A) The sequence of S100A8 promoter indicating the distance from TSS containing the IAA potential binding motif (pink) and p53 binding site (box) (top). The analysis of luciferase activity for the p53 KO microglia transfected with the luciferase plasmid inserted S100A8 promoter sequence. Treatment of IAA and/or recombinant mouse p53 protein is indicated in the figure (bottom). (FIG. 5B) Schematic diagram of the IAA binding motif forming a hairpin structure with complementary sequences. IAA promotes the unwinding of the hairpin exposing the transcription factor p53 binding site (box). (FIG. 5C) IAA binding oligo and mutant synthesized with modification of fluorescence Cy3 at the 50 and the Quencher BHQ1 30-end. DNA (20 mM) unwound with helicase (100 ng/mL) and/or IAA. The fluorescence value was recorded at an excitation wavelength at 550 and emission wavelength at 570. (FIG. 5D) Biotinylated IAA incubated with shearing BV2 genomic DNA. The interaction of IAA and the promoter of S100A8 indicated using the ChIP assay. (FIG. 5E) SPR analysis of interaction between p53 protein and biotinylated oligo covalently immobilized onto the sensor chip with or without IAA. (FIG. 5F) Biotinylated oligo S100p1-G (WT) and mutant transfected into microglia cells for 12 h and incubate with mouse gut supernatant for additional 6 h. Metabolite analysis with LC-MS after pull-down with streptavidin beads. (FIG. 5G) KO p53 with CRISPR-Cas9 transfection for 48 h and the expression of S100A8 in microglia after treatment with IAA for 12 h analyzed by western blot. (FIG. 5H) 3D predicted structures of interaction between IAA and oligo S100p1-G (G, Red; T, Yellow; C, Green; A, Cyan) at positions G-4 and G-11 by two hydrogen bonds. (FIG. 5I) Multisequence alignment of S100A8 orthologues on promoter using CLUSTAL 2.1. The depth of color shading indicates the degree of residue conservation. Shown are the S100A8 orthologs for Mus musculus (SEQ ID NO: 279), Homo Sapiens (SEQ ID NO: 280), Gorilla gorilla (SEQ ID NO: 281), Bos Taurus (SEQ ID NO: 282), Equus caballus (SEQ ID NO: 283), and Orycteropus afer (SEQ ID NO: 284). (FIG. 5J) Phylogenetic tree of S100A8 orthologs on promoter sequences containing the IAA-binding motif using R package “ape” in a R 4.0 environment. The horizonal lines are branches and represent evolutionary lineages changing over time. The length of a branch represents genetic variation among these sequences. Data are representative of three independent experiments (error bars, SD). *p<0.05, **p<0.01 (two-tailed t test).

FIGS. 6A-6J. IAA promotes cognitive decline, whereas phage^{TSD-27} reverses cognitive decline. (FIG. 6A) Schematic diagram of the treatment schedule and timeline for learning and memory tests. Administration of IAA (0.5 g/kg) and phage^{TSD-27} (1×10⁹ pfu/each) to young and aged mice (n=10 each group), respectively, for 2 months prior to the behavioral tests. (FIG. 6B) Schematic diagram of the Morris water maze (MWM) test indicating a variety of insertion points (S, south; SW, southwest; W, west; NW, northwest; N, north). (FIG. 6C) Representative search paths taken by mice on the day of test at the insertion point of west (W, top) and south (S, bottom). (FIG. 6D) MWM performance of all groups of mice in the test (day 5). The aged mice revealed a greater path length per pool diameter as well as a longer escape latency compared with young mice. Administration of IAA and phage^{TSD-27} to young and aged mice, respectively, for 2 months significantly altered the search path and escape latency (left); MWM performance of mice in the exploration (day 1) and acquisition trials (day 2 to day 4) (right). * Y-IAA versus Y-PBS; #Aged-phage^{TSD-27} versus Aged-PBS. (FIG. 6E) Schematic diagram of the T-maze spontaneous alternation (T-maze, TMSA) test (left); Quantification of T-maze spontaneous alternation in test day (right). (FIG. 6F) Schematic diagram of the 2-object novel object recognition (NOR) test (left); percentage of the times for the novel objects out of the total exploration times (right). (FIG. 6G) H&E-stained sections of the cerebrum, thalamus, cerebellum, hippocampus, and brain stem (4003 magnification, scale bars, 200 mm). Arrows in the left indicate neuronal loss and dying neurons in aged mice (n=5 per group). (FIG. 6H) A representative 10 s clip of epidural electroencephalographic (EEG) captured from four electrodes (left) in the freely behaving mice (n=5 per group) in a wakened state. Different EEG channels are colored. Each EEG channel is identified with a two-letter label indicating its position: F, frontal; O, occipital; L, left; and R, right (middle). Density spectral array (DSA) showing the distribution of EEG strength in relation to frequency over time (right). The color scale represents power from 0 to 10 pW. (FIG. 6I) A heat map showing composition of EEG signal frequency in cerebral cortex of mice (n=5). Orange and blue colors represent high and low percentage of waveforms, respectively. (FIG. 6J) Represent analysis of western blot for the expression in microglia from different regions of the brain (left). The ratio of each band density to that of GAPDH was calculated and the relative expression levels normalized by cerebrum in the young mouse untreated group are shown. Red and blue colors represent elevated and decreased expression, respectively (right). Data are representative of five independent experiments (error bars, SD). *, #p<0.05, **, #, #p<0.01 (two-tailed t test).

FIGS. 7A-7I. S100p1-G mediated depletion of gut metabolite IAA leads to prevention of cognitive dysfunction. (FIG. 7A) Schematic diagram of IAA depletion (dep) from aged mice fecal supernatant (Sup) with biotinylated oligo S100p1-G, followed by pull-down with streptavidin beads. (FIG. 7B) IAA removed from the fecal supernatant of aged mice (12-month-old, male) with wild-type S100p1-G (IAA^{dep}) or mutant S100p1-G (Ctrl^{dep}) at 1.0 nM/mL. HPLC analysis of IAA in the supernatant (left); fecal supernatant with/without IAA depletion administered to 2-month-old male mice (n=6) via oral gavage and HPLC analysis of IAA in the serum collected 24 h after oral administration (right). (FIG. 7C) Young mice (2-month-old, male) were gavage-given TSD-27 bacterium (1×10⁹/mouse) along with phageTSD-27(1×10⁹ pfu/mouse) and phage^{LGG} (1×10⁹ pfu/mouse). A different group of young mice (2-month-old, male) were gavage-given-aged mouse-derived gut supernatant with IAA^{dep}, Ctrl^{dep} depletion (n=10 each group) or without depletion (−) as a control group. All mice were treated as described above every other day for 2 months prior to the behavioral tests and other tests (FIG. 7D-FIG. 7G). Representative search paths in theMWMtaken by mice on the day of test at the insertion point of west (W) (left). Quantification of relative search path taken by mice (right). (FIG. 7D) Quantification of T-maze spontaneous alternation. (FIG. 7E) Analysis of IAA in serum using HPLC. (FIG. 7F) Analysis of S100A8 in microglia of the brain using qPCR. (FIG. 7G) Analysis of S100A8 and cleaved caspase-3 in microglia of the brain by western blot. The color of the arrows represents the treatment group indicated in (C). (FIG. 7H) Oligo S100p1-G and S100p1-G mutant were intravenously administered to aged mice (12-month-old, male) via the tail vein at 1 mg/kg body weight (n=10) twice a week for 2 months prior to theMWMtesting. Representative search paths taken by mice in theMWMtest (left). Quantification of relative search path taken by mice (right). (FIG. 7I) HPLC analysis of IAA in CSF collected at 1 h after the last oligo S100p1-G and S100p1-G mutant treatments. Data are representative of five independent experiments (error bars, SD). *p<0.05, **p<0.01 (two-tailed t test).

FIG. 8. Timing starts the apoptosis in different brain cells from young to aged mice. The neuron, microglia, astrocytes and oligodendrocytes isolated from the brain of C57BL/6 mice at 2-month (m), 12 m, 18 m and 24 m old (n=10 each group). Analysis of apoptosis by flow cytometry using Annexin V-FITC and 7-AAD staining in brain cells. Numbers in boxes indicate a representative percent of apoptotic cells (left panel). Quantification of percentage of Annexin V⁺7-AAD⁻ cells (right panel). Data are representative of three independent experiments (error bars, SD). *p<0.05 and **p<0.01 (two-tailed t-test, 6 m, 12 m, 18 m and 24 m vs 2 m).

FIGS. 9A-9D. Comparative analysis of the sensome genes expressed in microglial cells from young and aged mice. (FIG. 9A) The microglial cells isolated from the cerebral cortex of C57BL/6 mice at 2 m (young) and 12 m (aged) old. The expression of select sensome genes analyzed with individual qPCR. Graphs show relative expression as fold-change after normalization by expression in the young group. (FIG. 9B) A qPCR analysis of microglial cells from the five separate regions of brain recovered from FACS sorting. The ratio of each gene to that of GAPDH was calculated, and the relative expression levels normalized by cerebrum in the young group are shown. Red and blue colors represent elevated and decreased expression of mRNAs, respectively. (FIG. 9C) Quantification of band density in western blot of FIG. 1D normalized to GAPDH. (FIG. 9D) Quantification of band density in a western blot normalized to GAPDH, FIG. 1F. *P<0.05; **P<0.01 (two-tailed t-test); Data are representative of three independent experiments (error bars, SD).

FIGS. 10A and 10B. HPLC analysis of IAA and CA. (FIG. 10A) HPLC analysis of IAA and CA in serum (left panel) cerebrospinal fluid (CSF) (right panel) of young and aged mice. (FIG. 10B) Rate of IAA and CA in serum and CSF. Data are representative of three independent experiments (error bars, SD). *p<0.05 and **p<0.01 (two-tailed t-test).

FIG. 11. S100A8 dependent induction of microglial cell caspase-3 activation. Microglial cells isolated from mice and transfected with S100A8 knockout (KO) plasmids pS100A8 CRISPR and control (Ctrl) plasmid. Western blot analysis of the expression of cleaved-caspase-3, total caspase-3 and GAPDH in microglia (left panel). Quantification of band density in western blot normalized to GAPDH (right panel). Data are representative of three independent experiments (error bars, SD); **P<0.01 (two-tailed t-test). NS, not significant (two-tailed t-test).

FIGS. 12A-12D. The effect of metabolite IAA and phage^{TSD-27} on the expression of S100A8 and apoptotic bodies released from microglia. (FIG. 12A) Western blot analysis of S100A8 and cleaved-caspase-3 in microglia from 2 m-old mice (young, n=5) pretreated with IAA (50 μM/d/kg), CA (100 μM/d/kg) or 12 m-old mice (aged, n=5) pretreated with TSD-27 targeting phage (phage^{TSD-27}) for two months via oral administration. Probiotic lactobacillus LGG targeting phage was used as a control. (FIG. 12B) Quantification of band density in western blot normalized to GAPDH. (FIG. 12C) Aged mice (n=5) administrated IAA or phage^{TSD-27} via oral gavage for two months and microglial cells isolated from mouse brains. The ABs isolated from the cell cultured medium after three days of culture. The ABs analysis by FACS. (FIG. 12D) Quantification of ABs with annexin⁺ in FACS. Data are representative of three independent experiments (error bars, SD); *P<0.05; **P<0.01 (two-tailed t-test).

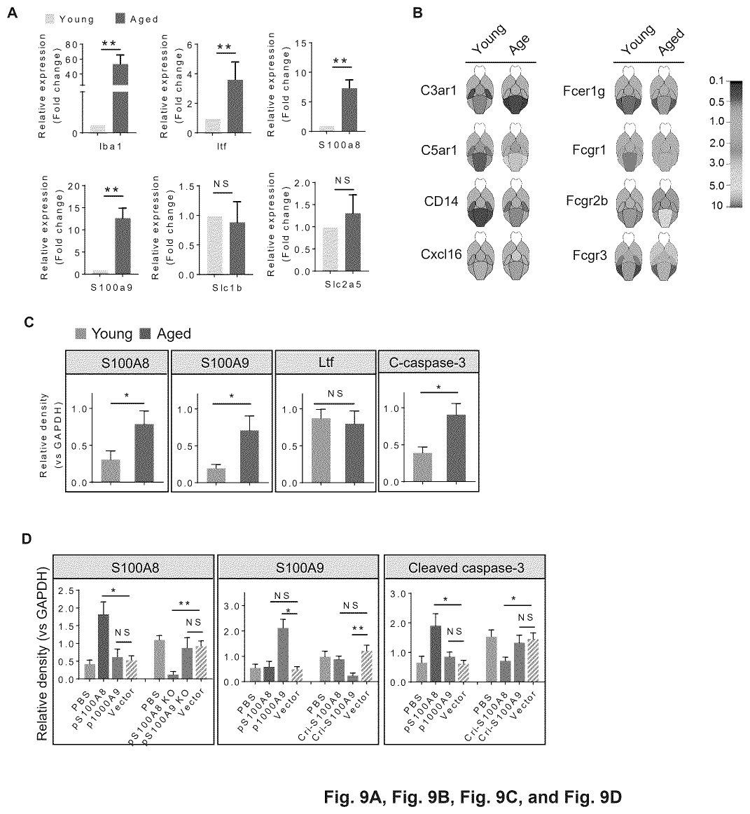

FIGS. 13A-13F. Identification of IAA binding motif on the promoter region of S100A8. (FIG. 13A) Sequence of mouse S100A8 (L76381)(SEQ ID NO: 13); CDS: 1038-2003; Transcription start-site (TSS) marked by a bent arrow. The sequences of synthetic oligos are underlined and the names are indicated below the sequence. The translation start code ATG is underlined and bolded. The sequences of the IAA binding motif are in red. Box, TATA box. (FIG. 13B) 10 pmol of synthetic DNA oligos S100p3-5 (top panel) and S100p6-7 (bottom panel) (60 mer/each) correspond to the sequence on the promoter of S100A8 incubated with IAA (1 M) for 30 min at 37° C. The oligos separated on 15% PAGE and visualized with ethidium bromide. (FIG. 13C) The synthetic DNA oligos S100p1-a to S100p1-h (10 mer/each) correspond to the sequence of S100p1. Representative 15% PAGE for the oligos incubated with IAA. (FIG. 13D) The shorter synthetic DNA oligos S100p1-A to S100p1-I (15 mer/each) correspond to the sequence of S100p1. Representative PAGE for the oligos S100p1-A to S100p1-E. (FIG. 13E) Microglial cells transfected with p53 CRISPR/Cas9 plasmid and western blot analysis of p53 expression. (FIG. 13F) 15% PAGE for the oligo of human S100A8 promoter sequence incubated with IAA. Data are representative of three independent experiments.

FIGS. 14A-14C. Oral administration of IAA and phage^{TSD-27} influences memory and learning loss. (FIG. 14A) HPLC analysis of IAA in serum 12 h after IAA administration and a week after phage^{TSD-27} gavage. The detail processing is in the legend of FIG. 4. (FIG. 14B) Mean velocity of mice in the MWM performance during the trials and test. The 2 m-old mice (young, n=5) pretreated with IAA (50 μM/d/kg) or 12 m-old mice (aged, n=5) pretreated with TSD-27 targeting phage (phage^{TSD-27}) for two months via oral administration prior to behavioral assays. (FIG. 14C) T-maze spontaneous alternation of mice under various treatment conditions. Data are representative of ten independent experiments (error bars, SD); *P<0.05; **P<0.01 (two-tailed t-test). In each of FIGS. 14A-14C, the bars from left to right correspond to young animals treated with PBS (Y-PBS), young animals treated with IAA (Y-IAA), aged animals treated with PBS (Aged-PBS), and aged animas treated with IAA after phage^{TSD-27} gavage (Aged-Phage^{TSD-27}).

FIG. 15. A schematic representation of electrodes and implantation preparation for EEG analysis. The vertical section of a craniotomy shows how an epidural electrode rests on the cranium with its tip contacting the dura surface.

FIG. 16. Oligo S100p1-G depletes the IAA from fecal supernatant. Fecal supernatant from aged mice with/without IAA depletion administered to young mice (n=5) via oral gavage and HPLC analysis of IAA in the CSF of mice 12 h after treatment of supernatant.

FIG. 17: Graphical abstract of a proposed model of promotion of p53-S100A8 signaling of microglia by gut bacterial metabolite IAA modulated by bacteriophage (phage).

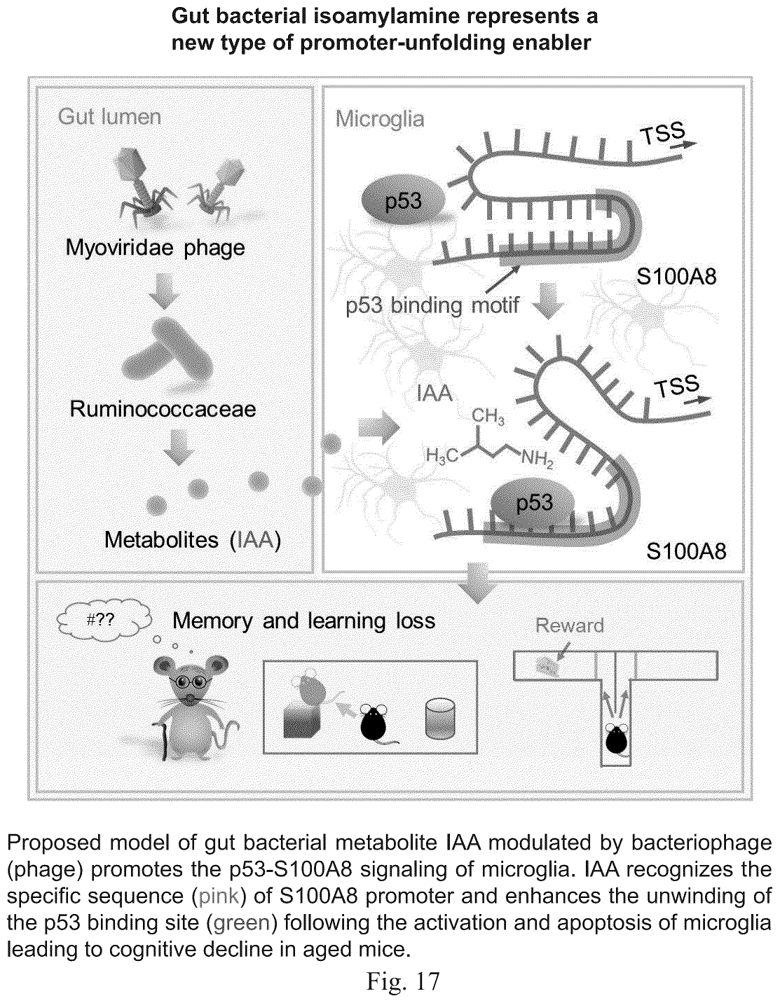

### DETAILED DESCRIPTION

The intestinal microbiome releases numerous different types of small molecules. Here, we show that the metabolite isoamylamine (IAA) released from gut bacteria Ruminococcaceae increases in aged mice and elderly people. Young mice orally administrated IAA leads to memory and learning loss. The IAA mediated induction of S100A8 promotes the apoptosis of microglial cells via recruiting p53 to S100A8 promoter region. IAA recognizes and specifically binds to this region and promotes the unwinding of its self-complementary hairpin structure that prevents the binding and activation by the transcription factor p53. The unwinding subsequently allows p53 to access the S100A8 promoter region and enhances the expression of S100A8. Thus, our finding provides evidence that small molecules released from the gut microbiome can directly bind genomic DNA, act as transcriptional coregulators, recruit sequence-specific transcription factors, and unveil a molecular mechanism that connects gut metabolism to gene expression in the brain and their implications for disease. While the following terms are believed to be well understood by one of ordinary skill in the art, the following definitions are set forth to facilitate explanation of the presently disclosed subject matter.

### I. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a method of the presently disclosed subject matter can “consist essentially of” one or more enumerated steps as set forth herein, which means that the one or more enumerated steps produce most or substantially all of the intended result to be produced by the claimed method. It is noted, however, that additional steps can be encompassed within the scope of such a method, provided that the additional steps do not substantially contribute to the result for which the method is intended.

As used herein, the term “and/or” when used in the context of a list of entities, refers to the entities being present singly or in any possible combination or subcombination. Thus, for example, the phrase “A, B, C, and/or D” includes A, B, C, and D individually, but also includes any and all combinations and subcombinations of A, B, C, and D.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### II. Exemplary Embodiments

There is no available approach to label metabolites released from all cells and gut microbiome to demonstrate the direct interaction between the promoter DNA region and metabolites, there is an inability to know whether metabolites regulate the accessibility of transcription factors to its regulated genes via direct binding to its motif region. Developing such technology is significant since unaccountable and numerous small molecules (metabolites) released from host cell and gut microbiomes regulate the expression of genes in host cells and play a physiological and pathophysiological role.

In one aspect, disclosed herein are methods of detecting the binding site of a small molecule to a target DNA sequence. Referred to herein as single-strand gel shift (SSGS) assay, the method comprises a) obtaining a short single-strand DNA oligonucleotide of a target DNA sequence; b) incubate DNA oligonucleotide with the small molecule creating a sample mixture; and c) performing nondenaturing polyacrylamide gel electrophoresis (PAGE) on the sample mixture, whereby a band on the gel is revealed for the sample mixture; wherein a shift in the band for the sample mixture relative to a control containing only the DNA oligonucleotide indicates the binding site for the small molecule to the target DNA sequence is present in the DNA oligonucleotide.

The SSGS assay can also be used to screen for small molecules (such as metabolites) that bind to a target nucleic acid and thus have broad applications for the discovery and identification of new medicaments. Thus, also disclosed herein are methods of screening for a small molecule that binds to a target DNA sequence, the method comprising a) obtaining a short single-strand DNA oligonucleotide of a target DNA sequence; b) incubate DNA oligonucleotide with the small molecule creating a sample mixture; and c) performing nondenaturing polyacrylamide gel electrophoresis (PAGE) on the sample mixture, whereby a band on the gel is revealed for the sample mixture; wherein a shift in the band for the sample mixture relative to a control containing only the DNA oligonucleotide indicates the small molecule binds the target DNA sequence.

The single-stranded DNA oligonucleotide used in the disclosed methods can be of any length suitable to exhibit conformational differences. Thus, in one aspect, the DNA oligonucleotide can be at least 10, 11, 12, 13, 14, 15, 16, 17, 18, 19, 20, 21, 22, 23, 24, 25, 26, 27, 28, 29, 30, 35, 40, 45, 50 or more nucleotides in length.

One small molecule (i.e., metabolite) interaction identified herein related to isoamylamine (IAA) and its target the promoter region of S100A8. As disclosed herein, we developed a simple and effective strategy with the single-strand gel shift (SSGS) assay to test our hypothesis that isoamylamine (IAA), a small molecule released from gut bacteria, binds to the promoter region of S100A8 and subsequently modulates the gene expression of S100A8.

We found that IAA and expression of S100A8 are both increased in an aging-dependent manner. We further discovered that prebinding of IAA to the promoter region of S100A8 is required for subsequently recruiting of p53, which in turn leads to inducing the expression of S100A8. Our findings reveal an unexpected role of IAA as a mediator of neurodegeneration elicited by IAA and p53 jointly activating S100A8 signaling of microglia leading to cognitive decline in aged mice and provide an example of how gut microbiome metabolites can now be applied to microglial cells to define mechanisms of neurodegeneration.

### II.A. Compositions

The presently disclosed subject matter relates in some embodiments to agents that, when administered to a subject such as but not limited to a mouse or a human sequesters isoamyl alcohol (IAA) produced by microbiota in the gut of a subject. Such an agent is referred to herein as an IAA sequesterer or an IAA sequestration agent. Any agent that is capable of sequestering IAA produced by microbiota in the gut of a subject can be employed in the compositions and methods of the presently disclosed subject matter.

In some embodiments, an IAA sequestration agent is an oligonucleotide, which in some embodiments is an oligonucleotide that has an IAA-binding sequence. By way of example and not limitation, an oligonucleotide that has an IAA-binding sequence can be a subsequence of an S100A8 genomic sequence, particularly a promoter sequence, that comprises an IAA-binding sequence. Exemplary S100A8 promoter include the human and mouse S100A8 promoters, fragments of which are disclosed herein as SEQ ID NOs: 12 and 13. In some embodiments, an IAA-binding sequence comprises, consists essentially of, or consists of the sequence 5′-TGGGCAGCTGGCCA-3′ (SEQ ID NO: 10), which is present in both the human and mouse S100A8 promoters. In some embodiments, an IAA-binding sequence comprises, consists essentially of, or consists of the sequence 5′-CTGTGGGCAGCTGGCCAAGC-3′ (SEQ ID NO: 11), which is also present in both the human and mouse S100A8 promoters.

Thus, in some embodiments the compositions of the presently disclosed subject matter comprise, consist essentially of, or consist of oligonucleotides that comprise, consist essentially of, or consist of the sequence 5′-TGGGCAGCTGGCCA-3′ (SEQ ID NO: 10) or the sequence 5′-CTGTGGGCAGCTGGCCAAGC-3′ (SEQ ID NO: 11). Other sequences that include an IAA-binding sequence are shown in Table 5 below, including but not limited to the oligonucleotides disclosed therein as S100p1, S100p1-G, and hS100A8p.

TABLE 5
Sequence of synthetic oligonucleotides in S100A8 promoter

| Name | Sequences (5′-3′) | Distance from TSS |
|---|---|---|
| S100p7 | TTCAGAGGTA GACTGGACAT GAAGACATTG GATCAGCAAT GGATCCAATT | −357 . . . −298 |
| | AGGAGAGAGC (SEQ ID NO: 14) | |
| S100p6 | AGGAGAGAGC TAAGATTGAG AGTCTGTTTA GATGCAGGGA TGAGGTGCCA | −307 . . . −248 |
| | GGGGCCTAGA (SEQ ID NO: 15) | |
| S100p5 | GGGGCCTAGA CATGGACTTA TTGAACTGCC CCATCCTGAT TCTTCCTGCT | −257 . . . −198 |
| | GGGTACTCCT (SEQ ID NO: 16) | |
| S100p4 | GGGTACTCCT GTCTGGTAAA TGTTCCAACA CTCCCACTTC CTCAGACTCA | −207 . . . −148 |
| | GAAATGCTCA (SEQ ID NO: 17) | |
| S100p3 | GAAATGCTCA CTGTACTCAG TGATTGCCAC ATGGACAAGG TTAGGAAACA | −157 . . . −98 |
| | GAGGCTGTGG (SEQ ID NO: 18) | |
| S100p2 | GAGGCTGTGG CAACTCTGGA AGGGAAGAGC GTTGTCTCCA TAGCCCGAGG | −107 . . . −48 |
| | CTGTGGGCAG (SEQ ID NO: 19) | |
| S100p1 | CTGTGGGCAG CTGGCAAGC TTTCCTCTAT AAAAGCAGCT GACACTTAGC | −57 . . . −1 |
| | CTCACAT (SEQ ID NO: 7) | |
| S100pla | CGAGG CTGTG (SEQ ID NO: 20) | −60 . . . −50 |
| S100p1b | CTGTG GGCAG (SEQ ID NO: 21) | −55 . . . −45 |
| S100p1c | GGCAG CTGGC (SEQ ID NO: 22) | −50 . . . −40 |
| S100p1d | CTGGC CAAGC (SEQ ID NO: 23) | −45 . . . −35 |
| S100ple | CAAGC TTTCC (SEQ ID NO: 24) | −40 . . . −30 |
| S100p1f | TTTCC TCTAT (SEQ ID NO: 25) | −35 . . . −25 |
| S100p1g | TCTAT AAAAG (SEQ ID NO: 26) | −30 . . . −20 |
| S100p1h | AAAAG CAGCT (SEQ ID NO: 27) | −27 . . . −18 |
| S100p1i | CAGCT GACAC (SEQ ID NO: 28) | −20 . . . −10 |
| S100p1j | GACAC TTAGC (SEQ ID NO: 29) | −17 . . . −8 |
| S100p1k | TTAGC CTCAC (SEQ ID NO: 30) | −12 . . . −3 |
| S100p1-A | AAAAGCAGCT GACACTTAGC (SEQ ID NO: 31) | −27 . . . −8 |
| S100p1-B | TCTATAAAAG CAGCTGACAC (SEQ ID NO: 32) | −32 . . . −13 |
| S100p1-C | TTTCCTCTAT AAAAGCAGCT (SEQ ID NO: 33) | −37 . . . −18 |
| S100p1-D | CAAGCTTTCC TCTATAAAAG (SEQ ID NO: 34) | −42 . . . −23 |
| S100p1-E | CTGGCCAAGC TTTCCTCTAT (SEQ ID NO: 35) | −47 . . . −28 |
| S100p1-F | GGCAGCTGGC CAAGCTTTCC (SEQ ID NO: 36) | −52 . . . −33 |
| S100p1-G | CTGTGGGCAG CTGGCCAAGC (SEQ ID NO: 8) | −57 . . . −38 |
| S100p1-H | CGAGGCTGTG GGCAGCTGGC (SEQ ID NO: 37) | −62 . . . −43 |
| S100p1-I | TAGCCCGAGG CTGTGGGCAG (SEQ ID NO: 38) | −67 . . . −48 |
| S100p1-G-Mut | CTGTATGCAA TTGGCCAAGC (SEQ ID NO: 9) | |
| hS100A8p | AGCTGTGGGCAGCTGGCCAAGCCTAA (SEQ ID NO: 39) | |

In some embodiments, an oligonucleotide that comprises, consists essentially of, or consists of an IAA-binding sequence can further comprise a tag and/or a detectable label. An exemplary tag would be a biotin moiety. Any tag that can facilitate recovery or identification of the molecule can be employed. Representative tags are epitope tags (for example, myc tags, FLAG™ tags, His6 tags, VSV-G tags, HSV tags, V5 tags, or any other tag for which a reagent is available or can be produced to facilitate isolation of the molecule) and small molecules such as biotin. See e.g., Brenner & Lerner (1992) Proc Natl Acad Sci USA 89:5381-5383; U.S. Pat. No. 6,068,829.

### II.A.1. Formulations

The compositions of the presently disclosed subject matter can be administered in any formulation or route that would be expected to deliver the compositions to whatever target site might be appropriate.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

### II.A.2. Dosages

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in seizure activity and/or in the incidence of death, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, a pharmaceutically or therapeutically effective amount of a IAA sequestration agent are delivered to the subject.

### II.A.3. Routes of Administration

Suitable methods for administration of the compositions of the presently disclosed subject matter include, but are not limited to intravenous administration, oral delivery, and delivery directly to a target tissue or organ (e.g., the gut, topical application). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sufobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

In some embodiments, the mode of administration is a solid dosage form, such as tablets and pills that are orally administered.

### II.B. Methods and Uses

The compositions of the presently disclosed subject matter can be employed in any method or use for which sequestration of IAA could be desirable. In some embodiments, the presently disclosed subject matter thus relates to methods for reducing and/or delaying cognitive decline associated with neurodegenerative disease in a subject in need thereof. In some embodiments, the methods comprise, consist essentially of, or consist of administering to a subject in need an effective amount of a composition comprising, consisting essentially of, or consisting of an IAA sequestration agent, including but not limited to the oligonucleotides disclosed herein that comprise, consist essentially of, or consist of an IAA-binding sequence. As used herein, the phrase “reducing and/or delaying cognitive decline associated with neurodegenerative disease” refers to reducing and/or delaying any cognitive decline in the subject that is associated with (i.e., is a consequence of) a neurodegenerative disease in the subject as compared to the cognitive decline in the subject that would have occurred had the IAA sequestration agent not been administered to the subject.

In some embodiments, a neurodegenerative disease for which the compositions and methods of the presently disclosed subject matter would be appropriate is a neurodegenerative disease associated with activation of the TLR4 pathway, optionally activation of the TLR4 pathway in the brain. Although not wishing to be bound by any particular theory of operation, because IAA can induce expression of S100A8, which is involved in the TLR4 pathway, activation of the TLR4 pathway in brain, in some embodiments by IAA, can contribute to neurodegenerative disease and/or neurodegeneration. Exemplary neurodegenerative disease associated with TLR4 pathway activation include, but are not limited to stroke, ischemic reperfusion injury, age-related dementia, Alzheimer's disease (AD), Parkinson's disease (PD), and amyotrophic lateral sclerosis (ALS).

Thus, in some embodiments the presently disclosed subject matter relates to methods for sequestering isoamyl alcohol (IAA) produced by microbiota in the gut of subjects. In some embodiments, the methods comprise, consist essentially of, of consist of administering to a subject in need thereof an effective amount of a composition an IAA sequestration agent, including but not limited to the oligonucleotides disclosed herein that comprise, consist essentially of, or consist of an IAA-binding sequence. As used herein, the phrase “sequestering isoamyl alcohol (IAA)” refers to any methodology wherein IAA produced by microbiota in the gut of a subject is removed from the subject's circulation or otherwise prevented from interacting with biological molecules present in the subject, particularly wherein those interactions would have negative consequences for the subject had the sequestration not taken place. An exemplary negative consequence is cognitive decline.

In some embodiments of the presently disclosed methods, the composition is administered orally.

In some embodiments of the presently disclosed methods, the composition is administered intravenously. In some embodiments, it is noted that any route of administration can be employed provided that the IAA sequestration agent can be delivered to a target in the subject, wherein the target is one where undesirable interactions between IAA produced by microbiota in the gut of the subject and a target cell, tissue, or organ occur.

In some embodiments, the subject is a mouse or a human.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the Examples

Mice. Given women are more resilient than men in age-related cognitive decline studies, as well among men, mean age at first incidence of cognitive impairment is earlier than women, we used only male mice in this study. 6-8 weeks old (young group), 12-14 months old (middle aged group), 18-20 months old and 22-24 months old (aged group) male specific pathogen-free (SPF) C57BL/6 mice were purchased from the Jackson Laboratory (Bar Harbor, ME). 6-8 weeks old and 12-14 months old male C57BL/6 syngeneic germ-free (GF) mice were purchased from the National Gnotobiotic Rodent Resource Center (University of Northern Carolina, NC Chapel Hill, NC) and maintained in flexible film isolators (Taconic Farm) at the Clean Mouse Facility of the University of Louisville. Animal care was performed following the Institute for Laboratory Animal Research (ILAR) guidelines and all animal experiments were done in accordance with protocols approved by the University of Louisville Institutional Animal Care and Use Committee (IACUC, Louisville, KY). The mice were acclimated for at least 1 week before any experiments were conducted.

Clinical Samples. The study involved 24 healthy volunteers divided into a young group (25.6±4.2 years old, n=12) and an older group (65.3±5.6 years old, n=12) by age. The participants in the two groups were matched for age and gender. All clinical fecal samples from healthy volunteers were collected in the Department of Surgery, Huai'an First People's Hospital, Huai'an, Jiangsu, China with written informed consent from patients. Approval for the study was granted by the Institute Research Ethics Committee at the Health Department of Huai'an. All subjects provided signed informed consent to this study. Volunteers were recruited from the population in 2017 in Huai'an, Jiangsu, China. No subjects had a history of chronic gastrointestinal disease, taking antibiotics within three months of testing, alcohol abuse, or smoking.

Cell culture. The C57BL/6 syngeneic microglia BV2 cell line (American Type Culture Collection, Rockville, MD) was grown at 37° C. in 5% C₀2 in RPMI 1640 medium (Gibco), supplemented with 10% heat-inactivated fetal bovine serum (FBS), 100 U/mL penicillin, and 100 g/ml streptomycin.

Bacteria. Ruminococcaceae sp. (ATCC, TSD-27) was grown in brain heart infusion (BHI) medium at 37° C. in a BactronEZ SHEL LAB anaerobic chamber (Sheldon Manufacturing, Inc. Cornelius, OR). Lactobacillus rhamnosus GG (LGG, ATCC #53103, Manassas, VA) was grown in De Man, Rogosa and Sharpe (MRS) broth (Hardy Diagnostics, Santa Maria, CA) and incubated in anaerobic conditions also.

Isolation of microglia, neuron, astrocytes, and oligodendrocytes from the brain. Mice were anaesthetized with a mixture of ketamine (16%) and xylazine (3%) by intraperitoneal injection and then sacrificed under basal conditions by transcardial perfusion with perfusion buffer (Ca²⁺/Mg²⁺ free HBSS containing 0.5 mM EGTA, 10 mM HEPES and 4.2 mM NaHCO₃; pH 7.2). The perfused brain was removed and gently mechanically disaggregated with tweezers in dissociation buffer (HBSS containing 10 mM HEPES and 4.2 mM NaHCO₃ supplemented with Type I collagenase (0.05%), Dispase II (25 mg/ml), DNase I (2.5 mg/ml), trypsin inhibitor (50 g/ml); pH 7.5) followed by incubation at 37° C. for 1 hour. The tissue homogenate was centrifuged at 1,350×g for 5 minutes at 4° C. For the isolation of microglia, the digestion enzymes in the tissue homogenate were inactivated by addition of PBS/EDTA containing 5% fetal bovine serum (FBS) and the digested brain bits were triturated gently, passed over a 100 m filter (Fisher Scientific) and centrifuged. Cell pellets were resuspended in 10 ml RPMI/L glutamine, mixed gently with 4.5 ml of physiologic PERCOLL® (Sigma Aldrich), and centrifuged at 850×g for 40 minutes. The pellet of cells was rinsed in PBS. Any contaminating red blood cells (RBC) were lysed using RBC lysis buffer (Sigma) according to the manufacturer's instructions, rinsed twice with PBS and passed through a 40 m filter (Fisher Scientific). Cells were then stained for CD11b and CD45 for flow cytometry analysis. The astrocytes were isolated from mouse cortices (n=10). The tissue homogenate was centrifuged at 800×g for 10 minutes at 4° C. following digestion with type II collagenase (1 mg/ml) and type I DNase (10 U/ml) in DMEM for 10 minutes. The cells pelleted by centrifugation were plated in a 100 mm culture dish coated with poly-L-ornithine (Sigma). For the isolation of oligodendrocytes, the glial cells above were cultured in specific media containing T3 and T4 (0.5 mM) as well as platelet-derived growth factor (PDGF, 10 ng/ml) to promote the growth of the different cell types. Isolated glial cells were labelled with the following antibodies against surface markers: CD11b (Biolegend, Cat #101206), CD45 (Biolegend, Cat #147710), GFAP (Biolegend, Cat #837508) and MOG (Biorbyt, Cat #orb412341-FITC). Cells positive for CD11b and CD45Med were identified as microglia. Cells positive for GFAP and negative for CD45 and CD11b were identified as astrocytes. Cells sorted with MOG+CD11b− were identified as oligodendrocytes. Neurons were isolated from mouse brain using the Pierce Primary Neuron Isolation Kit (Thermo Scientific, Cat #88280). Briefly, the tissue homogenate from freshly dissected cortexes was suspended in an equal volume of ice cold HBSS (Sigma) and replaced with Neuronal Isolation Enzyme (with papain) in a 37° C. incubator for 30 minutes. After washing with HBSS, the tissue was cultured in a poly-D-lysine coated plate with Neuronal Culture Medium.

Isolation of phage from gut feces. One gram of fecal sample was weighed and homogenized in 40 mL of PBS with a benchtop homogenizer (MP Biomedical). The sample was then centrifuged at 17,000×g for 5 min and the supernatant was filtered sequentially through a 2 m and 0.45 m filter. Phages were then concentrated using a polyethylene glycol (PEG) method. Briefly, one molar solid NaCl and 10% (v/v) PEG 8000 (Sigma) were dissolved in PBS and incubated overnight at 4° C. as recommended for a constant and stable precipitation. The phages mixed in the solution were pelleted by centrifugation at 5,250×g for 1 hour at 4° C. and re-suspended in 500 μl of SM buffer (NaCl 100 mM, MgSO4·7H₂O 8 mM, Tris-Cl 50 mM).

Phage DNA extraction and sequencing. Isolated phages were treated with 10 U/ml of DNase (Sigma) for 30 min at 37° C. followed by 10 min at 65° C. to inactivate the DNase. DNA was then extracted using the QIAamp DNA Microbiome Kit (Qiagen, 51704). Phage DNA was sequenced with the Illumina MiSeq Nano V2 PE_250 bases method using the Nextera XT Kit with an input of 1 ng DNA. All samples were sequenced using paired-end reads (numbers of reads mentioned in this manuscript always refer to paired-end reads). To identify viral genomes, raw sequencing fastq files were first converted to fasta files and duplicate sequences were removed using SeqKit v2.1.0. Then the viral genomes were searched to the NCBI complete RefSeq of viral sequences using BLASTn. Only perfectly matched sequences and high similarity of E-value<10-10 from BLASTn search were selected for further analysis. For taxonomic classification, sequences were assigned to different viral families according to ICTV-defined families and Virus-Host DB. Sequences assigned to multiple families were excluded and only sequences assigned to a unique family were used. Composition of taxonomic families were calculated based on the number of reads assigned to each family.

Microbiota 16S rRNA gene sequencing. Microbial genomic DNA from fecal samples was isolated with QIAamp DNA Stool Mini Kits (Qiagen), and bacterial strains were investigated using 16S rRNA gene sequencing. DNA (15 ng) was used as a template to amplify the 16S rRNA gene using a High Fidelity PCR system kit (Roche). The v1-v3 regions of 16S ribosomal RNA gene were amplified using 27f (AGAGTTTGATCCTGGCTCAG; SEQ ID NO: 1) and 534r (ATTACCGCGGCTGCTGG; SEQ ID NO: 2) primers (1 μM). The primers were anchored with adaptors (adaptor A: 5′-CCATCTCATCCCTGCGTGTCTCCGACTCAG-3′; SEQ ID NO: 3) and adaptor B: 5′-CCTATCCCCTGTGTGCCTTGGCAGTCTCAG-3′; SEQ ID NO: 4) and Multiplex Identifiers (MIDs; 10 bp long). The multiplexed amplicons were purified using a QIAquick Gel Extraction Kit (Qiagen). The amplicon sequence was conducted using the 454 Jr. Sequencing platform. The 16S rRNA gene sequences were analyzed using QIIME platform scripts. The microbial classification was performed with the GreenGenes reference data base (gg_otus-13_8) using QIIME tools. By applying hierarchical clustering algorithms (HCAs), we determined the species clustering based on the operational taxonomic unit (OTU) using amplicon sequencing of 16S RNA. The reference sequences allowed sorting of the results into OTUs by clustering 97% sequence similarity (uclust) and classification according to various taxonomic ranks (phylum, order, class, family, genus, and species). The percentage of each bacterial species was virtualized with R software.

Quantitative Real-Time PCR (qPCR) analysis of mRNA expression. Total RNA was isolated from tissues and cells with a RNeasy mini kit (Qiagen). For analysis of gene mRNA expression, 1 g of total RNA was reverse transcribed by SuperScript III reverse transcriptase (Invitrogen) and quantitation was performed using primers (Eurofins) with SSOADVANCED™ Universal SYBR Green Supermix (BioRad); GAPDH was used for normalization. The primer sequences are listed in Table 3. qPCR was run using the BioRad CFX96 qPCR System with each reaction run in triplicate. Analysis and fold-change were determined using the comparative threshold cycle (Ct) method. The ratio of each gene to that of the internal control was calculated, and the relative expression levels are shown in the Figures. Gene expression was normalized to the control expression by calculating the Δ Ct=(Ct of control−Ct of the gene). Setting the expression value of GAPDH to 1.0, the relative expression values were calculated as 2ΔCt. The change in mRNA expression was calculated as fold-change.

Quantification of bacteria and phages by qPCR. For gut bacteria identification, qPCR was performed from gut microbiota-derived DNA extracted with QIAamp DNA Stool Mini Kit (Qiagen). All kits were used according to the manufacturer's instructions. Quantitation was performed using SSOADVANCED™ Universal SYBR Green Supermix (BioRad) and the bacterial specific primers listed. qPCR was run using the BioRad CFX96 qPCR System with each reaction run in triplicate. Analysis and fold-change were determined using the comparative threshold cycle (Ct) method.

Analysis of microbial metabolites using liquid chromatography-mass spectrometry (LC-MS). Fecal samples were suspended in PBS (1 g/ml). After centrifugation at 10,000×g for 10 min, the supernatant was collected for LC-MS analysis. LC-MS was carried out using a method as described in. Proteome Discoverer v1.4.1.114 (Thermo) was used to analyze the data collected by the mass spectrometer. The database used in Mascot v2.5.1 and SequestHT searches was the Feb. 17, 2017 version of the bacterial metabolites from UniprotKB (Proteome ID UP000000955). Scaffold was used to calculate the false discovery rate using the Peptide and Protein Prophet algorithms. Proteins were grouped to satisfy the parsimony principle. The proteins were clustered based on differential expression and heat maps representing differentially regulated proteins by GELNs were generated using software R.

High performance liquid chromatography (HPLC) analysis of microbial metabolites. The fecal samples and TSD-27 growth medium were diluted with an equal volume of methanol. After centrifugation at 10,000×g for 30 minutes, 50 μl of supernatant was used for HPLC analysis. The HPLC analysis was performed on an Agilent 1260 Infinity system equipped with an Agilent ZORBAX SB-C18 column (4.6×150 mm, 3.5 μm), having the following parameters: mobile phase A: 5 mM NH₄Ac in water modified with 0.1% formic acid (v/v); mobile phase B: 5 mM NH₄Ac in 90% acetonitrile modified with 0.1% formic acid (v/v); gradient: 5% B in first 5 minutes, 5-20% B for 10 minutes, hold 20% B for 5 minutes, 20%-50% B for 5 minutes, hold 50% B for 5 minutes, 50%-100% B for 5 minutes, hold 100% B for 10 minutes, 100-5% B for 5 minutes; flow rate: 1.0 ml/min; temperature: 30° C. FLD (ex=280 nm, em=350 nm) was used for detection of isoamylamine (IAA), crotonic acid (CA) and pyridoxine. The standard for IAA (cat #: 126810) and CA (cat #: 113018) were purchased from Sigma.

Interaction of S100p1-G and metabolite. Microglial BV2 cells (2×10⁶) were transfected with biotinylated oligo S100p1-G or mutant using Lipofectamin RNAiMAX Reagent (Thermo Fisher) according to the manufacturer's instructions overnight at 37° C. 100 ml of aged mouse fecal supernatant (from one-gram feces) was added into the cell growth medium. After six hours, the cells were collected in RIPA lysis buffer. 100 mg of cell lysate was incubated with 50 ml of DYNABEADS® Streptavidin beads (Thermo Fisher) at room temperature for 1 h. The bio-oligo complex was eluted with biotin (4 mg/ml)) in 25 mM Tris-HCL containing 0.3 M NaCl (PH 8.5) at 95° C. for 5 minutes. Metabolite analysis was performed with LC-MS.

Flow cytometry. Isolated cells were incubated with blocking solution (0.5% BSA/PBS) for 15 minutes at 4° C. For surface staining, cells were stained with various antibodies at room temperature for 1 hour and then washed with PBS. Washed cells were stained for 40 minutes at 4° C. with the appropriate fluorochrome-conjugated antibodies in PBS with 2% FBS. Data were acquired using a BD FACSCalibur flow cytometer (BD Biosciences, San Jose, CA) and analyzed using FlowJo software (Tree Star Inc., Ashland, OR). The following antibodies purchased from Biolegend were used for flow cytometry: anti-CD11b (101208), anti-CD45 (103112) and FITC Annexin V (640945). For the ABs analysis, extracellular vesicles were collected. To recognize the ABs, as described previously, size gates set based on forward scatter (FSC) were applied in combination with Annexin V staining. From these vesicles total ABs were detected using Annexin V staining events.

Isolation of apoptotic bodies. Apoptotic bodies (ABs) were isolated from culture supernatants as previously described. Briefly, cell culture medium was harvested and cells were removed by pelleting at 335×g for 10 minutes. To remove cell-debris, supernatants were centrifuged at 1,000×g for 10 minutes, followed by another centrifugation at 2,000×g for 30 minutes to pellet ABs. Pelleted ABs were resuspended and washed with PBS.

Western blotting. Cells were treated as indicated in individual Figure legends and whole cell extracts (WCE) were prepared in the radioimmunoprecipitation assay (RIPA) buffer (Sigma) with addition of protease and phosphatase inhibitors (Roche). Proteins were separated by 10% SDS-PAGE and transferred to polyvinylidene difluoride (PVDF) membranes (Bio-Rad Laboratories, Inc., Hercules, CA). Dual color precision protein MW markers (BioRad) were separated in parallel. Antibodies were purchased as follows: cleaved-caspase-3 (ab214430), cleaved PARP (ab203467) and GAPDH (ab9657, Abcam) from Abcam; S100A8 (47310) and S100a9 (72590) from Cell Signaling; and Ltf (GTX81085) from GeneTex. The secondary antibodies conjugated to Fluors Alex-488 (A32723) or Alex-594 (A11012) were purchased from Invitrogen (Eugene, OR). The bands were visualized on the Odyssey Imager (LiCor Inc, Lincoln, NE).

Histological analysis. For hematoxylin and eosin (H&E) staining, tissues were fixed with buffered 10% formalin solution (SF93-20; Fisher Scientific, Fair Lawn, NJ) overnight at 4° C. Dehydration was achieved by immersion in a graded ethanol series of 70%, 80%, 95%, and 100% ethanol for 40 minutes each. Tissues were embedded in paraffin and subsequently cut into ultra-thin slices (5 μm) using a microtome. Tissues were deparaffinized by xylene (Fisher) and rehydrated by decreasing concentrations of ethanol and PBS. Tissue sections were stained with H&E and slides were scanned with an Aperio ScanScope. For frozen sections, tissues were fixed with periodate-lysine-paraformaldehyde (PLP) and dehydrated with 30% sucrose in PBS at 4° C. overnight. The sections were incubated overnight at 4° C. with anti-Iba1 (FujiFilm, #019-19741) and anti-CD-11b (Biolegend, #101204) diluted 1:100. The signal was visualized with the secondary antibodies conjugated to Fluors Alex-488 or Alex-594 (Invitrogen) and nuclei were stained with 4′,6-Diamidino-2-phenylindole dihydrochloride (DAPI). The slides were scanned using an Aperio ScanScope or visualized using confocal laser scanning microscopy (Nikon, Melville, NY).

Apoptosis analysis by terminal deoxynucleotidyl transferase dUTP nick end labeling (TUNEL). Formalin-fixed mouse lung tissues were embedded in paraffin and sectioned. TUNEL was used to detect apoptosis in the sections placed on slides according to the manufacturer's protocol. Tissue sections were analyzed to detect localized green fluorescence (GFP) of apoptotic cells using an in situ Cell Death Detection kit (Roche) and DAPI blue fluorescence to detect cell nuclei. The signal was visualized using confocal laser scanning microscopy (Nikon, Melville, NY).

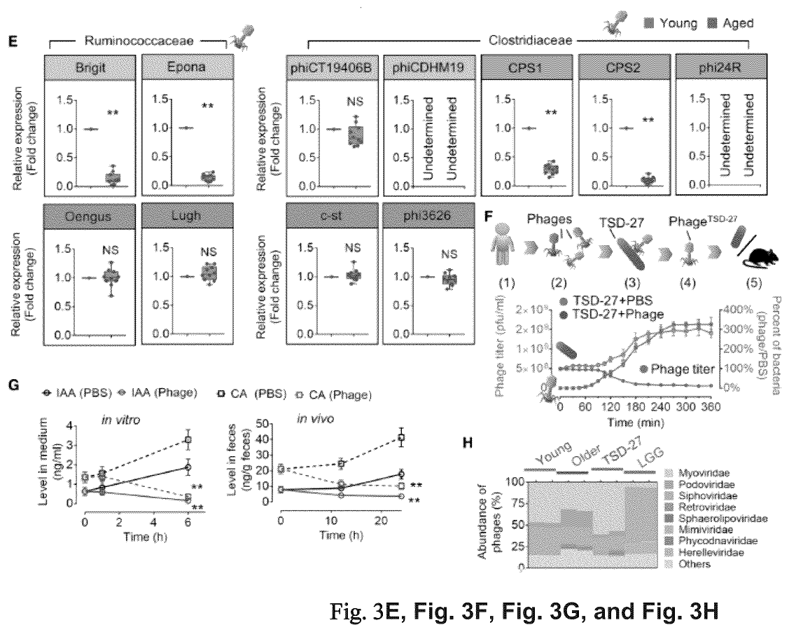

Morris water maze (MWM) test. Mice were trained in a 122 cm diameter, 50 cm height open-field water maze with non-reflective interior surfaces. The pool water was filled with water maintained at 25±1° C. and mixed with non-toxic white tempera paint. Distinctive and extra-maze cues were marked on the wall at specific locations and were visible to the mice in the maze. Mice were removed from cages, placed into the water from quasi-random start points and allowed a maximum of 60 seconds to probe the escape platform. The inter interval of each trail remained for 15 seconds. For four consecutive days, four trials (training) were recorded each day with a final test on day 5 (FIG. 4A). Performance was evaluated by the mean escape latencies base on every training trial (Vorhees and Williams, 2006).

Novel object recognition (NOR) test. During the habituation phase, mice were allowed free exploration of a 40×40 cm open-field box for 5 min. The arena was thoroughly cleaned with 70% (v/v) ethanol between mouse use. On the first training run, two identical objects were placed in opposite quadrants of the box and the mice were allowed to familiarize themselves with the objects for 10 min. After 24 h, one of the objects was replaced by a novel one of different color and shape but in in the same location. Mice were removed from the cages and placed in the box. The time that mice spent in discovery of the familiar and the new object was recorded over a 10 min period. For four consecutive days, four trials (training) were recorded each day with a final test on day 5 (FIG. 4A). The three- or four-day intersession interval is used to assess recognition memory performances.

T-maze test. To evaluate the cognitive impairment without requirement of full motor function, modified T-Maze test (Deacon and Rawlins, 2006) was carried out. Mice were gently handled, habituated to the T-Maze apparatus, and subjected to food deprivation. On the forced alternation sample trial (T1), the animals were exposed to the T-maze with one of the arms opened and rewarded with pellet at end of the arm. The opposite arm was blocked by a guillotine door. The mouse was allowed to explore the open arm and consume the reward. Ten consecutive trials were performed, then the mouse was removed. After 30 min, retrieval testing (T2) was performed. The blocked arm was opened and the mouse was placed in the same start position. If the mouse entered the previously blocked arm the response was recorded as “correct” and the animal received a reward. If the mouse entered the same arm as in T1, the mouse was confined for 10 seconds and this response was marked as an “Error”. Each mouse was subjected to 10 consecutive runs. Forced Alternation [%] was defined as the percent of mice first entering the novel arm during T2. For four consecutive days, four trials (training) were recorded each day with a final test on day 5 (FIG. 4A). The three- or four-day intersession interval is used to assess recognition memory performances.

Electroencephalography (EEG) recording in mice. Groups of mice were anesthetized by peritoneal administration of ketamine (90 mg/kg) and xylazine (10 mg/kg) mixture. Mice that were not responsive to nociceptive stimuli were consider anesthetized and were administered the analgesic buprenorphine (0.1 mg/kg). The fur of the skull area was removed, placed on surgical bench and skin area were cleaned with betadine and 70% ethyl alcohol. The midline scalp skin was lifted using forceps and an incision made from the frontal cranial bone to the back of the parietal cranial bones. The skin was push aside and cleaned with betadine using cotton swabs. The skull was cleaned and marked for placement of electrodes. Two craniotomies 0.25-0.5 mm in diameter on the side of each hemisphere were established a 3 mm distance from the midline vertical fissure in the frontal and occipital regions; a hand-held drill (Kopf) with matching drill bits (0.5 mm) was used for the craniotomies. Four electrodes were placed in appropriate skull holes over the frontal cortex (FC) and occipital cortex (OC). A ground/reference electrode was also implanted in the most anterior region of cerebellum. Thus, two independent EEG channels were created for FC and OC on each side of the hemisphere. Tissue adhesive (Vetbond) was applied to fix the electrodes on the skull and dental acrylic cement (Fixodent ultra) was used to cover the entire open skull area to secure the electrodes. The mice were allowed to wake-up in a clean cage pre-warmed on a heating pad. EEG recordings were conducted spontaneously and simultaneously for 10-20 minutes when the subjects were awake using the Cascade PRO IONM system (Cadwell). EEG signals were amplified 1,000× and digitized at 1 kHz. Data was collected using a standard PC computer (Optiplex GX620, Dell) running CASCADE® IDNM software (Cadwell).

Single-strand gel shift (SSGS) assay. To identify the metabolite binding site of S100A8, we synthesized a series of oligonucleotides (oligos) (Eurofins Genomics LLC, KY) specific to the response putative IAA binding site at the promoter region of S100A8. The sequences of the oligos are listed in Table 5. The oligos (10 μmol) were incubated with 1 nM of IAA at 37° C. for 30 minutes following electrophoresis on a 15% native PAGE without SDS in 0.5×TBE buffer. The oligos were stained with ethidium bromide (0.5 g/ml) and visualized under ultraviolet (UV) light.

Surface plasmon resonance (SPR). To identify the binding activity of the metabolite IAA and S100A8 promoter sequence, SPR experiments were conducted on an OPENSPR™ (Nicoya, Lifesciences, CA). Experiments were performed on a streptavidin sensor (Nicoya, Lifesciences). SPR was run at a flow rate of 20 μl/min using HBS running buffer (20 mM HEPES, 150 mM NaCl, pH 7.4). First, the streptavidin sensor chip was cleaned with octyl β-D-glucopyranoside (40 mM) and CHAPS (20 mM). 200 μl of biotin-conjugated oligos (1 μg/ml, Table 5) were injected on the sensor chip for 10 minutes until a stable resonance was obtained. After immobilization of bio-oligo, the surface was blocked with BSA (3%) in running buffer. After a stable signal was obtained, IAA (10 nM) was run over the immobilized bio-oligo until a stable resonance was obtained. A negative control test was also performed by injecting IAA onto a blank sensor chip to check for non-specific binding. The sensograms were analyzed using TraceDrawer kinetic analysis software.

Chromatin immunoprecipitation assay. To identify the interaction of IAA and promoter of S100A8, 1 mg of IAA was labeled with biotin using the EZ-Link NHS-PEG4 Biotinylation kit (Thermo Fisher Sci., #21455) and excess biotin was removed using a desalting column. Biotinylated IAA was incubated with microglia BV2 cells for 3 h at 37° C. following 37% formaldehyde (final 1%) in 1 ml PBS for one more hour for cross-linking. The cell lysate was collected in 1 ml of RIPA buffer and sonication was used to shear the chromatin into 0.5˜1 kb fragment. Streptavidin Dynabeads (50 ml) were added to the DNA samples for 1 h at room temperature. PBS replaced the DNA and was incubated with the beads as the control to exclude non-specific interactions. After washing beads with PBS, biotinylated IAA was eluted from the beads with the biotin (4 mg/ml) in 25mMTris-HCL containing 0.3MNaCl (pH 8.5) as the elution buffer at room temperature for 1 h or 95° C. for 5 min. The supernatant was collected and the DNA was purified with acetic acid (2%, v/v) and dissolved into 10 ml of H2O. Two ml of DNA was used in quantitative PCR reactions using the primers of S100A8-ChIP in Table 3.

Construction of the S100A8 promoter into the luciferase plasmid vector. To determine the activity of IAA on the promoter of S100A8, a sequence containing 1,037 bp of S100A8 promoter (GENBANK® Accession #: L76381) upstream −1,037 to −1 from transcription start site (TSS) was constructed into the luciferase C1 vector (Addgene, #163523). A DNA fragment was generated by PCR amplification with genomic DNA extracted from mouse liver tissue using DNeasy Blood & Tissue Kits (Qiagen, #69504). PCR was conducted using Q5 High-Fidelity PCR kit (New England Biolabs® Inc.) with the primers 51008A-prom, pLuc163523 and genomic DNA, and luciferase C1 plasmid as the template, respectively. The primer sequences are listed in Table 3. The two PCR products purified using the Gel extraction kit (Qiagen) were seamlessly assembled into the pLuc-S100p using the NEBuilder HiFi DNA Assembly Cloning Kit (New England Biolabs® Inc.). The plasmid pLuc-S100p was transformed into DH5a and selected using kanamycin resistance. The DNA sequence was confirmed by DNA sequencing.

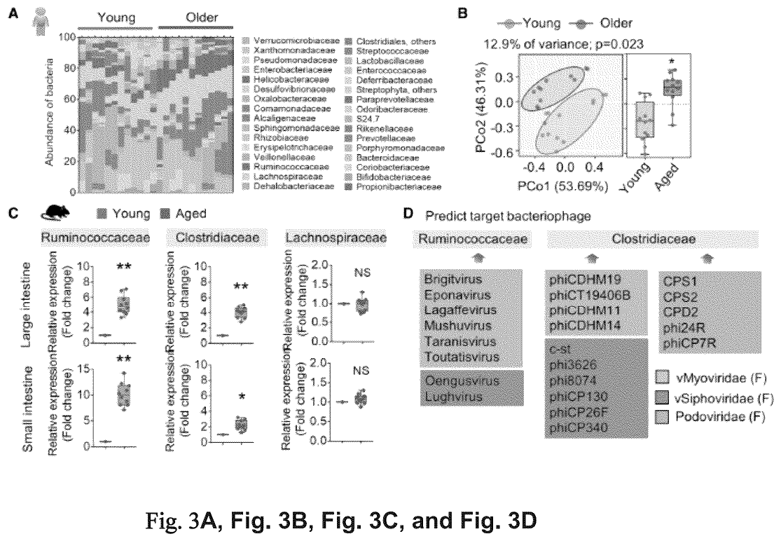

Site-directed mutagenesis within the S100A8 promoter. A mutant of IAA with the potential binding site on the S100A8 promoter was generated with the oligonucleotide primer S1008A-prom-Mut, which was designed to specifically disrupt the putative S100A8 binding site at its promoter (FIG. 3C). Q5® Site-Directed Mutagenesis Kit (New England Biolabs, MA, USA) was used in conjunction with specific primers (Table 3) to introduce S100A8 promoter mutations in the pLuc-S100p construct according to the manufacturer's instructions. After mutant strand synthesis and ligation, resultant plasmids were introduced into E. coli and transformants were selected using kanamycin resistance. The DNA sequence of mutants was confirmed by DNA sequencing.

TABLE 3
Primer sequences used for qPCR

| Primers | Forward (5′-3′) | Reverse (5′-3′) |
|---|---|---|
| Tnfα-1 | CACAGAAAGCATGATCCGCGA (SEQ ID NO: 234) | CGGCAGAGAGGAGGTTGACTT (SEQ ID NO: 235) |
| Iba1 | GTCCTTGAAGCGAATGCTGG (SEQ ID NO: 236) | CATTCTCAAGATGGCAGATC (SEQ ID NO: 237) |
| Gapdh | GGTCGGTGTGAACGGATTTG (SEQ ID NO: 238) | GGAGTCATACTGGAACATGTAG (SEQ ID NO: 239) |
| Ruminococcaceae | ACTGAGAGGTTGAACGGCCA (SEQ ID NO: 240) | CCTTTACACCCAGTAAWTCCGGA (SEQ ID NO: 241) |
| Clostridiaceae | GCACAAGCAGTGGAGT (SEQ ID NO: 242) | CTTCCTCCGTTTTGTCAA (SEQ ID NO: 243) |
| Lachnospiraceae | CGGTACCTGACTAAGAAGC (SEQ ID NO: 244) | AGTTT(C/T)ATTCTTGCGAACG (SEQ ID NO: 245) |
| 16S Universal | CTCCTACGGGAGGCAGCAG (SEQ ID NO: 246) | GTATTACCGCGGCTGCTG (SEQ ID NO: 247) |
| Brigitvirus | TTCTGCCGCTCGTATCT (SEQ ID NO: 248) | GAGCTGCATCTTCTACCTTG (SEQ ID NO: 249) |
| Oengusvirus | GTGAGCTACGAGACCTATCA (SEQ ID NO: 250) | GGCAAAGGGTCTCTTCATAC (SEQ ID NO: 251) |
| Lugh | CCGTTACCTGAGTGACAAAG (SEQ ID NO: 252) | CTGCCTTACTGCGTTTCA (SEQ ID NO: 253) |
| Siphov-Ring | GAGGACCGTGTAAAGGAATG (SEQ ID NO: 254) | CGAGCACTACCTGACTAAAC (SEQ ID NO: 255) |
| Siphov-Fury | CCCAGAGCAACTGTTGAAG (SEQ ID NO: 256) | GTGATCGAGGACTTCATCTTG (SEQ ID NO: 257) |
| Siphov-c-st | CCGTAGGATATGCTCAAACTTA (SEQ ID NO: 258) | GTTCCTCCTGCCATTGTT (SEQ ID NO: 259) |
| Podov-CPS1 | TTGTTACGGCATATGCTAGA (SEQ ID NO: 260) | GCTGTCAGTATCGCTGTATAA (SEQ ID NO: 261) |
| Podov-CPS2 | GGGTGGGACGTTGAATTT (SEQ ID NO: 262) | TCCGTTACTGTCTGATAGGG (SEQ ID NO: 263) |
| Siphov-phi3626 | GCAGGAGCATCAGTTGATAA (SEQ ID NO: 264) | GCAAGTAGCCCATAGTCATC (SEQ ID NO: 265) |
| Myo-phiCDHM19 | TCCTCCGATACATCCAAGAT (SEQ ID NO: 266) | GTCGTGCTGTTCTCGTTT (SEQ ID NO: 267) |
| Myo- | GCAAAGAGCTCCTCAATACA (SEQ ID NO: 268) | CACTCATTTGGTCGCATTTC (SEQ ID NO: 269) |
| phiCT19406B | | |
| peu-miR2916 | GCCGACCAGGGATCGGTGG (SEQ ID NO: 270) | Universal primer (Qiagen) |
| RNU6 | CGCAAGGATGACACGCAAATTC | Universal primer (Qiagen) |
| | (SEQ ID NO: 271) | |
| S1008A- | TAGCCCATATATGGAGTTCCAAGCTTTTGTA | ATCTGACGGTTCACTAAACCATGTGAGGCTAAGTGTCA |
| promoter | AGATAATCGTGG (SEQ ID NO: 272) | GCTG (SEQ ID NO: 273) |
| pLuc163523 | GGTTTAGTGAACCGTCAGATC (SEQ ID NO: 274) | GGAACTCCATATATGGGCTATG (SEQ ID NO: 275) |
| S1008A- | AAGGTAAAGCTTTCCTCTATAAAAGC | CTGCCCACAGCCTCGGGCTA (SEQ ID NO: 277) |
| prom-Mut | (SEQ ID NO: 276) | |

Nucleic acid unwinding assay. Helicase reactions were carried out in triplicate in 96-well plates. To prepare DNA substrate, 20 μM single strand DNA (WT: 5′-Cy3-GTGGGCAGCTGGCCAA-BHQ1-3′ (SEQ ID NO: 5); Mutant: 5′-Cy3-GTATGCAGCTGGATAA-BHQ1-3′ (SEQ ID NO: 6; Eurofins Genomics LLC, KY) were denatured at 95° C. for 5 minutes in the reaction buffer (25 mM Tris HCl pH 7.5, 100 mM NaCl, 15 mM MgCl₂, 1 mM DTT, 0.1 mg/ml BSA) and cooled slowly to room temperature before add IAA (100 nM) and/or helicase (100 g/ml). After ATP (2 mM) was added to initiate the helicase activity, the fluorescence value was recorded using a microplate reader with an excitation wavelength of 550 and an emission wavelength of 570. BHQ: black-hole quencher.

IAA depletion from gut supernatant. To remove the IAA from gut fecal supernatant, biotin-binding oligo S1001p-G (1 nM/ml, Eurofins) was incubated with fecal supernatant (1 g feces/ml) in PBS for 1 hour at room temperature. 100 μl of Dynabeads MYONE™ streptavidin (Thermo fisher) were incubated with the oligo/supernatant for 30 minutes and the tube containing the solution was place on a magnet for 1 minute. The supernatant was collected and the IAA level was estimated by HPLC.

Quantification and statistical analysis. Unless otherwise indicated, all statistical analyses in this study were performed using SPSS 16.0 software (Gouda, 2015). The data are presented as values with standard deviation (as the mean±SD). The significance of differences in mean values between two groups was analyzed using the Student's t-test. Differences between individual groups were analyzed via one- or two-way ANOVA. Differences between percentages of bacterial composition were analyzed with a chi-square test. Differences were considered significant when the P-value was less than 0.05 or 0.01. A P value greater than 0.05 was considered non-significant (NS). Both animals and human subjects were randomly assigned to a control group and different experimental condition groups matched for age and sex using simple randomization. Double-blinded studies were used for animal and human subject studies. Unless otherwise indicated, the mice used in the in vivo study were male C57BL/6 mice. Using one-way ANOVA comparing up to four groups, for a power of 0.7, a large effect size (0.75) and a significance level of 0.05, the minimum sample size needed in each group was 4.992 (rounded=5; Festing and Aktnab, 2002). The reported “n” in animal and human studies represents the number of animals and human subjects. Data are representative of at least three independent experiments.

### Example 1

### Gut Bacterial IAA Induces S100A8 Leading to Microglial Cell Death

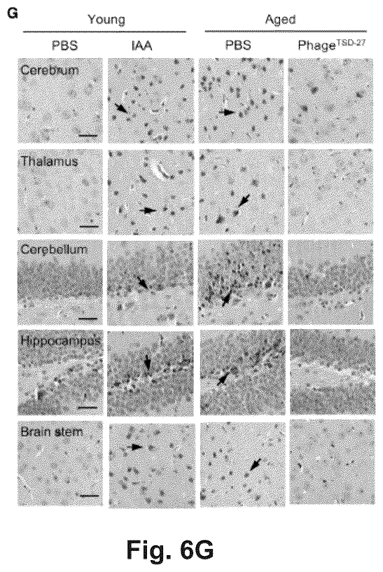

Given the death of brain cells including neuron, oligodendrocytes, microglia, and astrocytes is one of hallmark of cognitive decline in aging, and apoptotic cells (Annexin V+7−AAD⁻) were quantitatively analyzed from mice at different ages from 2 to 24 months. We found that microglia exhibited significant earlier apoptosis, starting in 12-month-old mice, whereas this phenomenon was not seen in neurons, astrocytes, and oligodendrocytes until approximately 18 months (FIG. 8). Microglia play a key role in memory retention and regulate the survivability of neurons; therefore, we focused our study on the cellular and molecular mechanisms underlying aging-related factors that contribute to microglial cell-mediated brain dysfunction and cognitive decline.

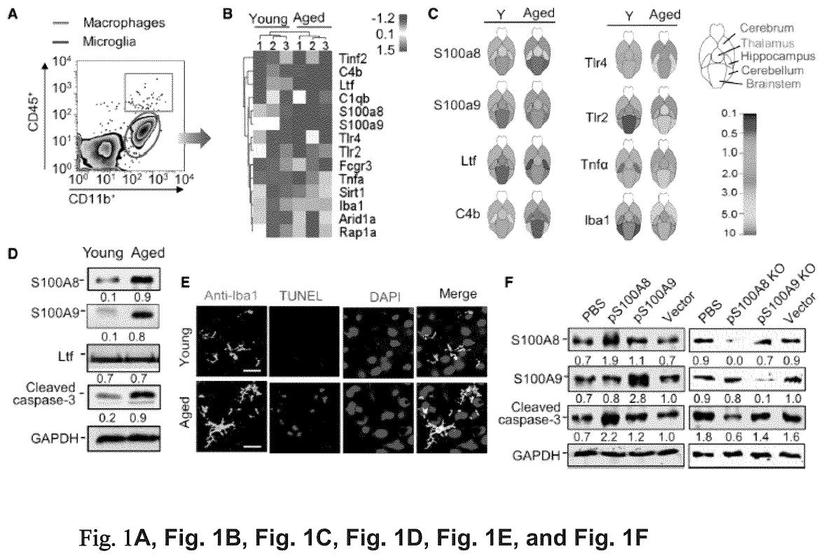

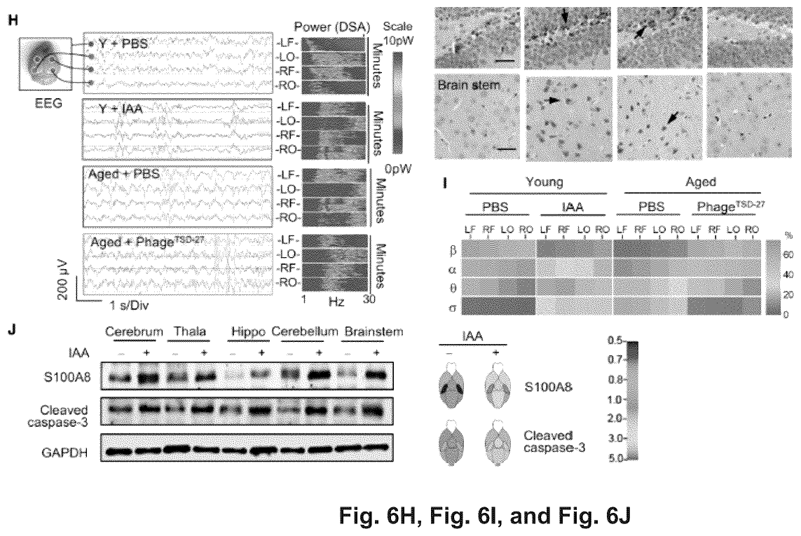

Microglia, the principal sentinels of the brain, sense changes in their environment and respond to invading pathogens, toxins, and cellular debris via expressing a cluster of genes that allow them to perform their sensing functions termed the sensome. With aging, the expression of sensome transcripts are altered. Thus, the sensome expressed in the microglia of brain from 2 months (young) and 12 months (aged) old mice was quantitively analyzed (FIG. 1A and Table 1). Fourteen of the sensome genes were increased in microglial cells of aged mice (FIG. 1B). In aged mice, the induction of 5100 calcium-binding protein A8 (S100A8), S100A9, ionized calcium-binding adapter molecule 1 (Iba1) and lactotransferrin (Ltf) in the microglia has been verified by individual qPCR (FIG. 9A). Considering the diverse roles and functions of the various regions of the central nervous system, it was necessary to determine the level of sensome expression in different regions of the brain (the cerebrum, thalamus, hippocampus, cerebellum, and brainstem). Interestingly, we found that the sensome expression is not evenly distributed in all regions of brain and exhibited diverse alterations in aged brain (FIG. 1C). The profile of sensome expression suggested that the sensome significantly induced in aged mice is S100A8 and C4b in the cerebrum; Ltf, S100A8, S100A9, and C4b in the cerebellum; S100A9 and C4b in the hippocampus; Ltf, S100A9, and C4b in the thalamus; Ltf, S100A8, S100A9, and C4b in the brainstem; TLR4 and TNF-a in hippocampus; and Iba1 in cerebrum and cerebellum (FIG. 1C). The diversity of more sensome expression in different regions of brain was demonstrated (FIG. 9B). Western blots confirmed the induction of S100A8 and S100A9 expression as protein in microglia (FIGS. 1D and 9C). The TUNEL assay suggested extensive apoptosis in Iba1+ cells in the brain of aged mice (FIG. 1E), which is consistent with the induction of cleaved caspase-3 in the microglia of aging (FIG. 1D).

TABLE 1
The microglial sensome genes with their ligands and primer sequences for qPCR

| Name | Ligand | Forward | Reverse |
|---|---|---|---|
| P2ry12 | Nucleotides | CACGGGATTCCCTACACCCTG | GGGTGCTCTCCTTCACCTAG |
| P2ry13 | Nucleotides-ADP | AACAAAGCTGATGCTCGGGA | GTGTCATCCGAGTGTCCCTG |
| P2ry6 | Nucleotides-UDP | CTGCGTCTACCGTGAGGATT | GCAATGACGCAGATGTTCAG |
| Gpr34 | Nucleotides, LysoPS | ACCGGATGGAAGAGCCCAGCTG | CAGTGTGACTTCTCAACGTCTTCT |
| Adora3 | Adenosine | TTGCTGGCCATTGCTGTAGA | GAGTGGTAACCGTTCTATATCTAC |
| Entpd1 | Nucleotides-ATP | AGCTGCCCCTTATGGAAGAT | TCAGTCCCACAGCAATCAAA |
| Tmem173 | Bacterial Cyclic | AGCGGAAGTCTCTGCAGTCT | GGAGCCCTGGTAAGATCAAC |
| | D [symbol] -GMP | | |
| Csflr | m-CSF, IL-34 | GCAGTACCACCATCCACTTGTA | GTGAGACACTCTCCTTCAGTGC |
| Csf3r | g-CSF | ACCCTTTGTGTTCCACCAGT | TTGGTCCTTTCTTCCTCCCT |
| Tgfbr1 | Tgf-β | TGCCATAACCGCACTGTCA | AATGAAAGGGCGATCTAGTGATG |
| Tgfbr2 | Tgf-β | CCTACTCTGTCTGTGGATGACCT | ACTTCCGGGGCCATGTAT |
| Ifngr1 | Tgf-γ | GCACTCGGTGGAATGAGACTATTG | GACCTGTCAGTTGATGCCTCAGAA |
| Il10ra | IL-10 | CAGGAACTGACGGATTGGGAA | GCTTCAAACCACACAGACGG |
| Il6ra | IL-6 | CCTCTGACTTCCATTTCTGCT | CAAGAATCGTCGTCCATGTCC |
| Il21r | IL-21 | CTCCCCCCTTGAACGTGACT | TTGCCCCTCAGCACGTAGTT |
| Tnfrsf17 | BAFF | GCCGACACCGAGCTGACTAG | CTTGCCGTAGTCACCCGTTT |
| Tnfrsf1b | TNF | GAGATGCCAAGGTGCCTCAT | AACTGGGTGCTGTGGTCAAC |
| Cx3cr1 | cx3cl1 | ACCGGTACCTTGCCATCGT | ACACCGTCCTGCACTGTCC |
| Ccr5 | Col3, Col4, Col8, | GGCAGGGCTCCGATGTATAA | CATCCGTTCCCCTACAAGAA |
| | RANTES | | |
| C3ar1 | C3a | CACCATGGAGTCTTTCGATGCTG | CACATCTGTACTCATATTG |
| Ptafr | FAF | CGAGGGCGACTGGATTCTAC | CACCCAAAAAGGCCACACTG |
| Gpr77 | C5a | CACACCACCAGCGAGTATTATG | AGCACAAGCAGGACTATCAGG |
| Cmklr1 | The chemokine chemerin | GCTTTGGCTACTTTGTGGACTT | CAGTGTTCACGGTCTTCTTCATC |
| Cysltr1 | LTD4 | CTCCAAGGCACCAAGCAGAC | TGCCAAAGAAACCCACAACAG |
| Ccr12 | Chemerin | TGTGTTTCCTGCTTCCCCTG | CGAGGAGTGGAGTCCGACAA |
| Cmtm6 | May bind cxcl7 | CTAGTATTCTTAATTCCTTCTGAGCC | AGCTTTGGCTCAGAAGGAATTAAG |
| C5ar1 | AKA CD88 C5a | GGCCATCCTGCGGCTGATGG | GCCTTGCGACTCCAGGTCCG |
| Fcgr3 | Fc portion of IgG | CCTCCATCTCTCTAGTCTGGTACCA | AGGCCCGTGTCCACTGC |
| Fcerlg | Fc portion of IgE | AAGATCCAGGTCCGAAAGGC | TCTGAAGCTACTGGGGTGGT |
| Fcgr2b | Fc portion of | GAAACCATCACGCTAAGGTGCC | TGGTGCAGTGTCCTTCCTAGAC |
| | IgG-Low affinity | | |
| Fcgr1 | Fc portion of | GCGGAAAGAGAAGATGCTGGATTC | CTTCTCTCTCTCCAGCCTGTGT |
| | IgG-high affinity | | |
| Cmtm7 | IgM | ACTATGGTGCCCACAGCTTC | AACCCAAAGATCGACAGGGG |
| Fcrl1 | IgG3 | CACACGGAGTAAGTGAGTCGT | TCAGGCCTTGGGCTTGTATG |
| Fcgr4 | Binds Fc fragment | TCCACCGTGGCATCAAATCA | GTCCTGAGGTTCCTTGCTCC |
| | of IgG | | |
| Selplg | Enterovirus 71, CD162 | GAAAGGGCTGATTGTGACCCCT | AGTAGTTCCGCACTGGGTACA |
| Ly86 | AKA MD1 LFS | CTGCCCTCCTTGTGTGGATTC | TGGAACACTGGTCAATGGAAAG |
| Cd68 | Ox LDL, SR | ACTGGTGTAGCCTAGCTGGT | CCTTGGGCTATAAGCGGTCC |
| Trem2 | Apoptotic neurons, | GCCTTCCTGAAGAAGCGGAA | GAGTGATGGTGACGGTTCCA |
| | Lipids, Aβ | | |
| Cd180 | LPS AKA cp105 | TAGGTCTCAATGAAATTCCTGGC | AATCTGGCACCTGGTTAAATCC |
| Tlr2 | Microbial | GCAAACGCTGTTCTGCTCAG | AGGCGTCTCCCTCTATTGTATT |
| Cd37 | P-glucan, dectin-1 | GCCCAAGAGAGTTGCCTCAG | GGCCGCCTATACAAAGAAGAA |
| Tlr7 | Pattern recognition | ATGTGGACACGGAAGAGACAA | GGTAAGGGTAAGATTGGTGGTG |
| | receptor | | |
| Cd14 | Pattern recognition | CTCTGTCCTTAAAGCGCCTTAC | GTTGCGGAGGTTCAAGATGTT |
| | receptor | | |
| Clec4a3 | HIV AKA DCIR3 | CTTCACTTCAACTGACTTGGTGG | TCACTGCTAGGCTCACCTTTG |
| Tlr4 | Microbial | TGTCATCAGGGACTTTGCTG | GGACTCTGATCATGGGACTG |
| Tlr13 | Bacterial 23 s rRNA | GTTGTAACCTGGATGCCTAAGAC | GGCCTCTGTCAAGTTGGTGA |
| Clec5a | Japanese encephalitis | TCGGGGCTTATCGTAGTAGTG | TGTAGGCATGGTACTTTCGTCAT |
| | virus | | |
| Haver2 | Unknown | TCAGGTCTTACCCTCAACTGTG | GGCATTCTTACCAACCTCAAACA |
| Clec7a | β glucan and yeast | GACTTCAGCACTCAAGACATCC | TTGTGTCGCCAAAATGCTAGG |
| Cxcl16 | Bacteria, OxLDL, MTB | CCTTGTCTCTTGCGTTCTTCC | TCCAAAGTACCCTGCGGTATC |
| Cd48 | MTB, E-coli | CCCAAGCCTTCCATAGAAATCAA | CCAAGTATAGTCAACATGCTGCT |
| Ltf | AGEs, Bacteria | GGAGCCTTGAGGTGTCTGAGA | AGGTGGCACTCCTTGTATTCTG |
| Cd74 | Bacteria (H. pylori), HIV | AGTGCGACGAGAACGGTAAC | CGTTGGGGAACACACACCA |
| Upk1b | Bacteria (E. coli) | CACTGTTCGTTGCTTCCAGG | GCTTCGAGAAGTGGGTAAAGACT |
| Tlr12 | Parasites 6 | GCCGCCATTCCAAGCTATC | CTCCACAGTCCGAGGTACAACTT |
| Tlr1 | Microbial | TCAAGTGTGCAGCTGATTGC | TAGTGCTGACGGACACATCC |
| Tlr6 | Microbial | TGGATGTCTCACACAATCGG | GCAGCTTAGATGCAAGTGAGC |
| Ifitm6 | May regulate viral entry | GGTTAAGAGGGATCC | CTTTGACAGTGCATG |
| Itgam | Fibrinogen, amyloid β | ATGGACGCTGATGGCAATACC | TCCCCATTCACGTCTCCCA |
| Itgb2 | CD18 binds amyloid β | CACTGTCTCAGTTGTGTACCAAG | GCTCTGGTGTATCACAGCGAA |
| Itgb5 | Adenovirus, Vitronectin | GAAGTGCCACCTCGTGTGAA | GGACCGTGGATTGCCAAAGT |
| Emr1 | AKA F4/80, adhesion78 | TCCTGCTGTGTCGTGCTGTTC | GCCGTGTGGTTGTCAGTCTTGTG |
| Ecscr | Filamin | AGCTGTGCTGGGTGATCCT | TTGTGGGCTGGGAGTTGT |
| Lair1 | Collagens | GCTCTGACCAGACCTGGTAAGG | CCATGTGTGTCTCCAGGTGTGC |
| Siglech | Sialic acid | ATTTCTGTGAGGAAAGGATC | AAT TCA CAGAAC TCC ACA GC |
| Slco2b1 | Anions | TTGGCATCGGTGGTGTGCCC | ATGCCTCCTTCTGGCATCCGGT |
| Slc2a5 | Glucose AKA Glut5 | GGCTCATCTTCCCCTTCATTCA | AATGCTCTGCCCTTGGTCTCTG |
| S100a8 | Find | CCGTCTTCAAGACATCGTTTGA | GTAGAGGGCATGGTGATTTCCT |
| S100a9 | Tin4 | ATACTCTAGGAAGGAAGGACACC | TCCATGATGTCATTTATGAGGGC |
| Lgals9 | Urate, glycans | CTGGAATCCCTCCTGTGGTGTA | CCTCGTAGCATCTGGCAAGACA |
| Gpr183 | Oxysterols | GTCGTGTTCATCCTGTGGTTCAC | TCATCAGGCACACCGTGAAGTG |
| Tmem37 | Inorganic cations? | CAGTGAACCACACTGTCTGAGAG | GGACACAATGAGCATCTCCAAGC |
| Cd33 | Siglec 3 | GATTCTTGGGGTCTGTCTCG | TGGACACTGCTCTGTTCCTG |
| Gpr84 | Fatty acids | GACTGCCCCTCAAAAGACCTGC | GCCACGCCCCAGATAATTGC |
| Slc7a7 | Amino acids | AAGGTGTTGGCGCTGATTGCAG | AGAGTGCCAGAGCAATGTCACC |
| Cd52 | Clq | ATCCTTGGGACAAGCCACTACG | CAGCTGAGGTAGAAGAGGCACA |
| Siglec5 | Sialic acid | AGCCCTTCCAGCTCTCTACC | GTCCCAGCAGCTGTAGAAGG |
| Cd79b | BCR complex | CCAGCAATGACAAGCAGTGACC | CCTGAGTGGTTTGTGTAGCAGTG |
| Slc16a3 | Monocarboxylate | TCCATCCTGCTGGCTATGCTCT | CAGAAGGACGCAGCCACCATTC |
| Icaml | LFA-1, rhinoviruses | CAATTTCTCATGCCGCACAG | AGCTGGAAGATCGAAAGTCCG |
| Icam4 | β2 Integrins | GGCCACAAGTACACTCTGC | GGCTTAAAGCGAGGACTGTCA |
| Cd84 | CD84 (Homophilic) | TTGTTCCGTTTGTTCAAGAG | CGGAATAAACTGTGTTCACTG |
| Lag-3 | Binds MHC II | CCAGGCCTCGATGATTGCTA | CAGCAGCGTACACTGTCAGA |
| Cd86 | CD28 on T cells | CATGGGCTTGGCAATCCTTA | AAATGGGCACGGCAGATATG |
| Ptprc | Galectin-1 | GGGTTGTTCTGTGCCTTGTT | CTGGACGGACACAGTTAGCA |
| Dap12 | Lipids | CCAGCCCCTGGACTGTGGTGTCC | GTACCGTGTGGATCTGTATTCCA |
| Tnfrsf13b | BLyS | AGCGCACCTGTGCAGCCTTCTG | GCCCCGGGAGAGCTGGACTTG |
| Tnfrsf17 | BAFF | CTAAGGAAGATAAACTCTGAACCA | TTACCTAGCAGAAATTGATTTCTC |
| Cd22 | CD45, IgM | CCACTCCTCAGGCCAGAAACT | TGCCGATGGTCTCTGGACTG |
| Tmem119 | AKA OEUF | GTGTCTAACAGGCCCCAGAA | AGCCACGTGGTATCAAGGAG |
| Cd53 | tetraspanin | TCCTCCTTGCTGAGGTGACC | AGGGTCAGTGCAAAGGACAT |
| Slamf9 | Unknown | CAGGGCTTTACAACGCCCAA | GTAGACACGGAGATGGTAAGACT |
| Clec4b1 | Unknown | CCACTGCTACTTGGTTCCCAC | TCCTCCTGGCTATGGATCACC |
| Lilra5 | Unknown | CGGAAGGGAATCCGCACAA | CACCTCACATGAGATGGTCAC |
| mGapdh | Control | GGTCGGTGTGAACGGATTTG | GGAGTCATACTGGAACATGTAG |
| mActb | Control | CTAAGGCCAACCGTGAAAAG | ACCAGAGGCATACAGGGACA |
| mRn18s | Control | GATCCATTGGAGGCAAGTCT | CCAAGATCCAACTACGAGCTTTTT |

[symbol]

indicates data missing or illegible when filed

Sequence identifiers for primers in Table 1: P2ry12 (Forward primer: SEQ ID NO: 40; reverse primer SEQ ID NO: 41), P2ryl3 (Forward primer: SEQ ID NO: 42; reverse primer SEQ ID NO: 4), P2ry6 (Forward primer: SEQ ID NO: 44; reverse primer SEQ ID NO: 45), Gpr34 (Forward primer: SEQ ID NO: 46; reverse primer SEQ ID NO: 47), Adora3 (Forward primer: SEQ ID NO: 48; reverse primer SEQ ID NO: 49), Entpd1 (Forward primer: SEQ ID NO: 50; reverse primer SEQ ID NO: 51), Tmem173 (Forward primer: SEQ ID NO: 52; reverse primer SEQ ID NO: 53), Csf1r (Forward primer: SEQ ID NO: 54; reverse primer SEQ ID NO: 55), Csf3r (Forward primer: SEQ ID NO: 56; reverse primer SEQ ID NO: 57), Tgfbr1 (Forward primer: SEQ ID NO: 58; reverse primer SEQ ID NO: 59), Tgfbr2 (Forward primer: SEQ ID NO: 60; reverse primer SEQ ID NO: 61), Jfngr1 (Forward primer: SEQ ID NO: 62; reverse primer SEQ ID NO: 63), I110ra (Forward primer: SEQ ID NO: 64; reverse primer SEQ ID NO: 65), I16ra (Forward primer: SEQ ID NO: 66; reverse primer SEQ ID NO: 67), I121r (Forward primer: SEQ ID NO: 68; reverse primer SEQ ID NO: 69), Tnfrsf17 (Forward primer: SEQ ID NO: 70; reverse primer SEQ ID NO: 71), Tnfrsf1b (Forward primer: SEQ ID NO: 72; reverse primer SEQ ID NO: 73), Cx3cr1 (Forward primer: SEQ ID NO: 74; reverse primer SEQ ID NO: 75), Ccr5 (Forward primer: SEQ ID NO: 76; reverse primer SEQ ID NO: 77), C3ar1 (Forward primer: SEQ ID NO: 78; reverse primer SEQ ID NO: 79), Ptafr (Forward primer: SEQ ID NO: 80; reverse primer SEQ ID NO: 81), Gpr77 (Forward primer: SEQ ID NO: 82; reverse primer SEQ ID NO: 83), Cmk1r1 (Forward primer: SEQ ID NO: 84; reverse primer SEQ ID NO: 85), Cys1tr1 (Forward primer: SEQ ID NO: 86; reverse primer SEQ ID NO: 87), Ccr12 (Forward primer: SEQ ID NO: 88; reverse primer SEQ ID NO: 89), Cmtm6 (Forward primer: SEQ ID NO: 90; reverse primer SEQ ID NO: 91), C5ar1 (Forward primer: SEQ ID NO: 92; reverse primer SEQ ID NO: 93), Fcgr3 (Forward primer: SEQ ID NO: 94; reverse primer SEQ ID NO: 95), Fcer1g (Forward primer: SEQ ID NO: 96; reverse primer SEQ ID NO: 97), Fcgr2b (Forward primer: SEQ ID NO: 98; reverse primer SEQ ID NO: 99), Fcgr1 (Forward primer: SEQ ID NO: 100; reverse primer SEQ ID NO: 101), Cmtm7 (Forward primer: SEQ ID NO: 102; reverse primer SEQ ID NO: 103), Fer11 (Forward primer: SEQ ID NO: 104; reverse primer SEQ ID NO: 105), Fcgr4 (Forward primer: SEQ ID NO: 106; reverse primer SEQ ID NO: 107), Se1p1g (Forward primer: SEQ ID NO: 108; reverse primer SEQ ID NO: 109), Ly86 (Forward primer: SEQ ID NO: 110; reverse primer SEQ ID NO: 111), Cd68 (Forward primer: SEQ ID NO: 112; reverse primer SEQ ID NO: 113), Trem2 (Forward primer: SEQ ID NO: 114; reverse primer SEQ ID NO: 115), Cd180 (Forward primer: SEQ ID NO: 116; reverse primer SEQ ID NO: 117), Tlr2 (Forward primer: SEQ ID NO: 118; reverse primer SEQ ID NO: 119), Cd37 (Forward primer: SEQ ID NO: 120; reverse primer SEQ ID NO: 121), Tlr7 (Forward primer: SEQ ID NO: 122; reverse primer SEQ ID NO: 123), Cd14 (Forward primer: SEQ ID NO: 124; reverse primer SEQ ID NO: 125), Clec4a3 (Forward primer: SEQ ID NO: 126; reverse primer SEQ ID NO: 127), Tlr4 (Forward primer: SEQ ID NO: 128; reverse primer SEQ ID NO: 129), Tlr13 (Forward primer: SEQ ID NO: 130; reverse primer SEQ ID NO: 131), Clec5a (Forward primer: SEQ ID NO: 132; reverse primer SEQ ID NO: 133), Havcr2 (Forward primer: SEQ ID NO: 134; reverse primer SEQ ID NO: 135), Clec7a (Forward primer: SEQ ID NO: 136; reverse primer SEQ ID NO: 137), Cxcl16 (Forward primer: SEQ ID NO: 138; reverse primer SEQ ID NO: 139), Cd48 (Forward primer: SEQ ID NO: 140; reverse primer SEQ ID NO: 141), Ltf (Forward primer: SEQ ID NO: 142; reverse primer SEQ ID NO: 143), Cd74 (Forward primer: SEQ ID NO: 144; reverse primer SEQ ID NO: 145), Upk1b (Forward primer: SEQ ID NO: 146; reverse primer SEQ ID NO: 147), Tlr12 (Forward primer: SEQ ID NO: 148; reverse primer SEQ ID NO: 149), Tlr1 (Forward primer: SEQ ID NO: 150; reverse primer SEQ ID NO: 151), Tlr6 (Forward primer: SEQ ID NO: 152; reverse primer SEQ ID NO: 153), Ifitm6 (Forward primer: SEQ ID NO: 154; reverse primer SEQ ID NO: 155), Itgam (Forward primer: SEQ ID NO: 156; reverse primer SEQ ID NO: 157), Itgb2 (Forward primer: SEQ ID NO: 158; reverse primer SEQ ID NO: 159), Itgb5 (Forward primer: SEQ ID NO: 160; reverse primer SEQ ID NO: 161), Emr1 (Forward primer: SEQ ID NO: 162; reverse primer SEQ ID NO: 163), Ecscr (Forward primer: SEQ ID NO: 164; reverse primer SEQ ID NO: 165), Lair1 (Forward primer: SEQ ID NO: 166; reverse primer SEQ ID NO: 167), Siglech (Forward primer: SEQ ID NO: 168; reverse primer SEQ ID NO: 169), Slco2b1 (Forward primer: SEQ ID NO: 170; reverse primer SEQ ID NO: 171), Slc2a5 (Forward primer: SEQ ID NO: 172; reverse primer SEQ ID NO: 173), S100a8 (Forward primer: SEQ ID NO: 174; reverse primer SEQ ID NO: 175), S100a9 (Forward primer: SEQ ID NO: 176; reverse primer SEQ ID NO: 177), Lgals9 (Forward primer: SEQ ID NO: 178; reverse primer SEQ ID NO: 179), Gpr183 (Forward primer: SEQ ID NO: 180; reverse primer SEQ ID NO: 181), Tmem37 (Forward primer: SEQ ID NO: 182; reverse primer SEQ ID NO: 183), Cd33 (Forward primer: SEQ ID NO: 184; reverse primer SEQ ID NO: 185), Gpr84 (Forward primer: SEQ ID NO: 186; reverse primer SEQ ID NO: 187), Slc7a7 (Forward primer: SEQ ID NO: 188; reverse primer SEQ ID NO: 189), Cd52 (Forward primer: SEQ ID NO: 190; reverse primer SEQ ID NO: 191), Siglec5 (Forward primer: SEQ ID NO: 192; reverse primer SEQ ID NO: 193), Cd79b (Forward primer: SEQ ID NO: 194; reverse primer SEQ ID NO: 195), Slc16a3 (Forward primer: SEQ ID NO: 196; reverse primer SEQ ID NO: 197), Icam1 (Forward primer: SEQ ID NO: 198; reverse primer SEQ ID NO: 199), Icam4 (Forward primer: SEQ ID NO: 200; reverse primer SEQ ID NO: 201), Cd84 (Forward primer: SEQ ID NO: 202; reverse primer SEQ ID NO: 203), Lag-3 (Forward primer: SEQ ID NO: 204; reverse primer SEQ ID NO: 205), Cd86 (Forward primer: SEQ ID NO: 206; reverse primer SEQ ID NO: 207), Ptprc (Forward primer: SEQ ID NO: 208; reverse primer SEQ ID NO: 209), Dap12 (Forward primer: SEQ ID NO: 210; reverse primer SEQ ID NO: 211), Tnfrsf13b (Forward primer: SEQ ID NO: 212; reverse primer SEQ ID NO: 213), Tnfrsf17 (Forward primer: SEQ ID NO: 214; reverse primer SEQ ID NO: 215), Cd22 (Forward primer: SEQ ID NO: 216; reverse primer SEQ ID NO: 217), Tmem119 (Forward primer: SEQ ID NO: 218; reverse primer SEQ ID NO: 219), Cd53 (Forward primer: SEQ ID NO: 220; reverse primer SEQ ID NO: 221), Slamf9 (Forward primer: SEQ ID NO: 222; reverse primer SEQ ID NO: 223), Clec4b1 (Forward primer: SEQ ID NO: 224; reverse primer SEQ ID NO: 225), Lilra5 (Forward primer: SEQ ID NO: 226; reverse primer SEQ ID NO: 227), mGapdh (Forward primer: SEQ ID NO: 228; reverse primer SEQ ID NO: 229), mActb (Forward primer: SEQ ID NO: 230; reverse primer SEQ ID NO: 231), and mRn18s (Forward primer: SEQ ID NO: 232; reverse primer SEQ ID NO: 233)

Although the role of S100A8/A9 in the regulation of amyloid peptide and Tau phosphorylation (Cristóvão and Gomes, 2019) has been reported, the relationship between S100A8/S100A9 and microglia death was not clear. We transfected CRISPR activation plasmid and CRISPR-Cas9 knockout (KO) plasmid to overexpress and KO the expression of S100A8 and S100A9 in microglial BV2 cells. We found that the cleaved caspase-3, a marker of a dying cell, was induced by the overexpression of S100A8 but not S100A9. Knockout of S100A8 but not S100A9 caused a reduction of cleaved caspase-3 (FIGS. 1F and 9D). These data suggested that induction of S100A8 contributes to microglial cell death.

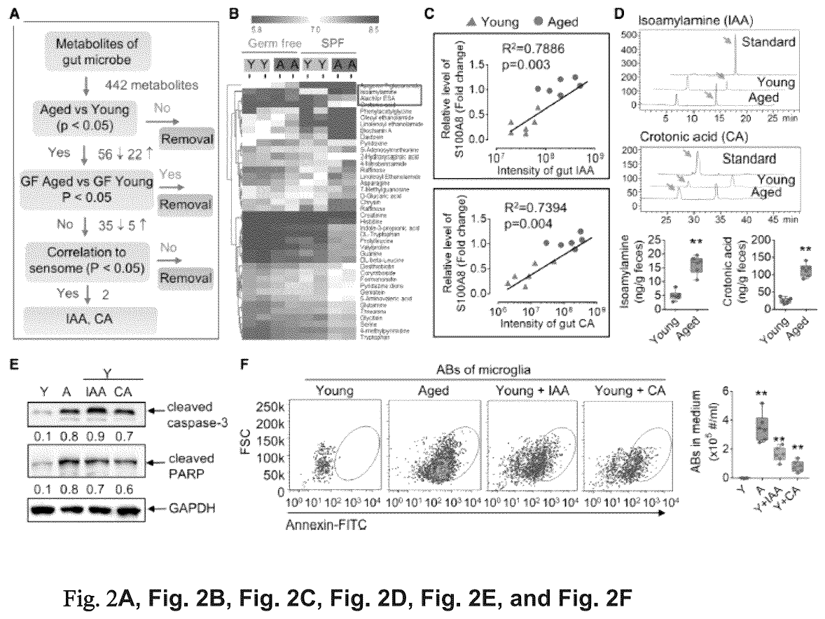

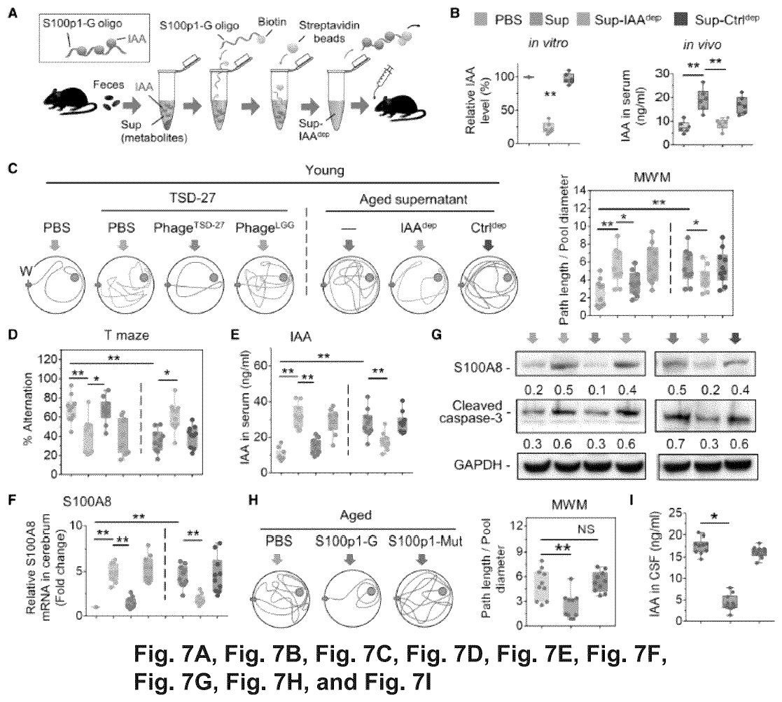

The gut microbiota can help to modulate homeostasis and behavior in its animal host through chemical communication with the nervous system. To determine whether gut microbe metabolites contribute to the modulation of microglia death in aging, we performed a comparative metabolomics analysis of the gut microbe from specific pathogen-free (SPF) and germ-free (GF) mice at different ages. A total of 442 microbial metabolites were identified in the murine gut based on the MetAboliC pAthways DAtabase for Microbial taxonomic groups (MACADAM) and MetaCyc database (FIG. 2A; Table 2). Based on excluding no significant change in SPF mice (aged versus young, p>0.05) and significant change in GF mice (aged versus young, p<0.05), 56 reductions and 22 inductions, and 35 reductions and 5 inductions were selected subsequently (FIGS. 2A and 2B). The five inductive metabolites, which included isoamylamine (IAA), alachlor ethanesulfonic acid (ESA), crotonic acid (CA), pyridoxine (B6), and S-adenosylmethionine, exhibited the most significant induction in SPF aged mice and no change in in the GF age matched mice when compared with appropriate young mice (FIG. 2B). A linear regression analysis was performed to identify which of the changes in metabolites was correlated with the expression of the S100A8. This analysis indicated that gut bacterial IAA and CA were significantly positively correlated with the expression of S100A8 expression in the aged mice brains (FIG. 2C). High-throughput LC-MS analysis of IAA and CA was further confirmed by high-performance liquid chromatography (HPLC) analysis (FIG. 2D), which was consistent with the high level of IAA and CA in the serum (FIG. 10A, left) and cerebrospinal fluid (CSF) (FIG. 10A, right) of aged mice. IAA was found to pass through the blood-brain barrier (BBB) in higher amounts than CA in aged mice (FIG. 10B) that may indicate that IAA plays a more critical role on the induction of S100A8 as well as influencing brain function compared with CA during the aging processing.

TABLE 2
Analysis of metabolites in mice gut bacteria using LC-MS

| | SPF mice | | | |
|---|---|---|---|---|
| Metabolites | Young1 | Young2 | Aged1 | Aged2 |
| 2-Amino-4-methylpyrimidine | 84404738.27 | 78835099.55 | 30218122.4 | 32326258.37 |
| Pyridazine-1,5-dione | 15316617.97 | 22785598.39 | 9187809.152 | 5385991.762 |
| 7-Methylguanocine | 10325154.4 | 3563465.968 | 3765358.995 | 2080735.34 |
| Apigenin 7-glucuronide | 12165.88481 | 197916.7749 | 1000 | 1000 |
| Biochanin A | 1148848.902 | 2419211.378 | 635226.3419 | 893477.1485 |
| Chrysin | 2993409.274 | 4717235.901 | 1128464.227 | 1205439.677 |
| Corymboside | 13252068.5 | 19190512.42 | 7480810.367 | 4804252.841 |
| Creatinine | 731092555 | 1087315642 | 314391993.2 | 360698238.9 |
| Desthiobiotin | 24838326.99 | 22255232.3 | 3701246.875 | 5822556.614 |
| Formononetin | 10729002.37 | 26029615.07 | 5322401.809 | 4786275.4 |
| Genistein | 15453988.95 | 22070719.33 | 7421740.062 | 8711208.34 |
| Glycitein | 51451952.42 | 80685401.75 | 20642378.42 | 25804113.4 |
| Guanine | 55302207.73 | 180214617.7 | 40042420.02 | 36423213.91 |
| Histidine | 432849718.8 | 429397622.7 | 149185610.3 | 172828936.4 |
| Indole-3 propionic acid | 170935770.7 | 163951865.9 | 26770263.45 | 30251247.65 |
| Isoamylamine | 10434802.36 | 4878324.982 | 265958447.7 | 175438013.9 |
| Linoleoyl Ethanolamide | 2643147.359 | 6975919.517 | 1278497.049 | 2238561.008 |
| Raffinose | 6225629.828 | 4892223.054 | 2939988.299 | 1630217.197 |
| 4-Nitrobenzamide | 16013593.75 | 14102275.49 | 13351516.91 | 1241763.269 |
| Oleoyl ethanolamide | 472637.653 | 1165705.223 | 33317.25727 | 139882.3734 |
| Phenylacetylglycine | 132664.8279 | 3595830.123 | 400437.4797 | 780066.6528 |
| Prolylleucine | 91861568.57 | 190533409.2 | 46726204.34 | 35815252.34 |
| Pyridoxine | 3493854.855 | 2815168.339 | 11135611.06 | 11771270.17 |
| 5-Adenosylmethionine | 13255315.16 | 12754164 | 24542374.47 | 50653297.78 |
| Serine | 45530689.8 | 42428481.92 | 19290481.29 | 21820729.18 |
| DL-Tryptophan | 233305688.4 | 208372103.9 | 36142274.13 | 38483982.71 |
| Valylproline | 114802358.9 | 115197051.2 | 35320787.84 | 42683137.8 |
| α-Linolenoyl ethanolamide | 169502.3359 | 645289.1522 | 143016.2255 | 132908.4915 |
| 2-Hydroxycaproic acid | 22239987.04 | 11317304.26 | 7132799.107 | 7441744.56 |
| 5-Aminovaleric acid | 18851806.36 | 19178234.41 | 6733917.279 | 9143914.131 |
| Alachior ESA | 765029.1671 | 1178517.482 | 2235763.173 | 2705101.287 |
| Asparagine | 7296943.658 | 9194260.827 | 2191507.401 | 2725158.108 |
| Crotonic acid | 844751.4472 | 1237164.214 | 8973361.409 | 3026714.664 |
| Daidzein | 2412297.684 | 3775079.404 | 739609.9242 | 83309.78306 |
| D-Glucaric acid | 5677535.817 | 11908692 | 2558604.178 | 2382951.73 |
| DL-β-Leucine | 121906701 | 99745333.25 | 40463409.15 | 53270588.51 |
| Glutamine | 34665111.14 | 35535924.32 | 14325971.45 | 15804029.32 |
| Raffinose | 1454794.538 | 3462111.191 | 1159277.654 | 1206310.188 |
| Threonine | 35012275.19 | 29093299.27 | 12076586.42 | 15940584.23 |
| Tryptophan | 80943307.12 | 52359796.82 | 21135041.7 | 29012221.55 |
| | GF mice | | | |
| Metabolites | Young1 | Young2 | Aged1 | Aged2 |
| 2-Amino-4-methylpyrimidine | 127371287.3 | 59393126.5 | 128351450.3 | 11426992.79 |
| Pyridazine-1,5-dione | 37952426.77 | 58447619.71 | 33619133.87 | 51866694.67 |
| 7-Methylguanocine | 15011251.33 | 2048942.043 | 15807134.25 | 2234630.309 |
| Apigenin 7-glucuronide | 8965415.977 | 8965415.977 | 8733835.395 | 6733835.395 |
| Biochanin A | 11209749.92 | 690481.2702 | 6314138.385 | 481249.4138 |
| Chrysin | 24103584.23 | 1472590.471 | 25880594.38 | 2375025.495 |
| Corymboside | 39655498.59 | 40149411.59 | 45784981.39 | 2952853.468 |
| Creatinine | 1295687950 | 407177885 | 996018095.9 | 57959544.18 |
| Desthiobiotin | 49002273.47 | 2788732.381 | 75507347.4 | 1581588.339 |
| Formononetin | 36603881.26 | 3902297.787 | 26983540.89 | 3128350.739 |
| Genistein | 34152833.92 | 7455528.487 | 20333390.13 | 925153.4661 |
| Glycitein | 75034880.85 | 21925533.77 | 45740759.65 | 15442914.3 |
| Guanine | 214900612.4 | 4394512.156 | 339417211.5 | 35841477.23 |
| Histidine | 665579489 | 309802300.2 | 658720599.3 | 58591551.84 |
| Indole-3 propionic acid | 320209921.9 | 29355719.37 | 351437302.5 | 13228318.05 |
| Isoamylamine | 1308949.341 | 400462.2989 | 925914.7515 | 445395.7817 |
| Linoleoyl Ethanolamide | 55640476.17 | 2538967.084 | 77985103.15 | 1257407.833 |
| Raffinose | 100284698 | 5132331.649 | 90520105.3 | 81265.42763 |
| 4-Nitrobenzamide | 2814940.175 | 13322961.84 | 23820048.62 | 2851772.77 |
| Oleoyl ethanolamide | 9630943.53 | 109143.5529 | 15894923.76 | 150903.9403 |
| Phenylacetylglycine | 776728.5454 | 4465673.093 | 3213951.294 | 129687.9111 |
| Prolylleucine | 428321193 | 20740090.02 | 834753490.5 | 10758463.5 |
| Pyridoxine | 7023533.985 | 3149972.063 | 2135542.638 | 3795183.573 |
| 5-Adenosylmethionine | 2027548.518 | 3198595.922 | 3288184.272 | 4237903.642 |
| Serine | 56151452.8 | 3877005.538 | 118120864.2 | 3374591.871 |
| DL-Tryptophan | 440896398.6 | 39102706.01 | 474479410.2 | 18437829.46 |
| Valylproline | 445857072.5 | 20125008.93 | 477784502.2 | 11744925.43 |
| α-Linolenoyl ethanolamide | 5098536.34 | 61822.15639 | 6627865.797 | 110685.7521 |
| 2-Hydroxycaproic acid | 2756093.465 | 9062472.545 | 3064470.677 | 10973179.18 |
| 5-Aminovaleric acid | 38428131.73 | 4397124.477 | 51480366.91 | 1802590.898 |
| Alachior ESA | 858810.7391 | 637604.589 | 1004484.343 | 713027.7226 |
| Asparagine | 54178272.64 | 5329630.946 | 50465637.92 | 591858.1314 |
| Crotonic acid | 228502.7124 | 7810550.633 | 7715318.694 | 7621387.454 |
| Daidzein | 7260168.662 | 1233016.591 | 5820296.656 | 28778646.432 |
| D-Glucaric acid | 9569484.706 | 10353395.72 | 14950155.21 | 274528.0753 |
| DL-β-Leucine | 205740865.7 | 11725551.99 | 278554166.7 | 9354570.123 |
| Glutamine | 75270614.87 | 4344800.37 | 88341414.52 | 3445597.443 |
| Raffinose | 25658055.86 | 3580674.007 | 15907949.27 | 108705.5347 |
| Threonine | 50955370.3 | 1709925.581 | 50358459.97 | 2612556.72 |
| Tryptophan | 129556819.9 | 8160538.945 | 139471049.5 | 4553985.35 |

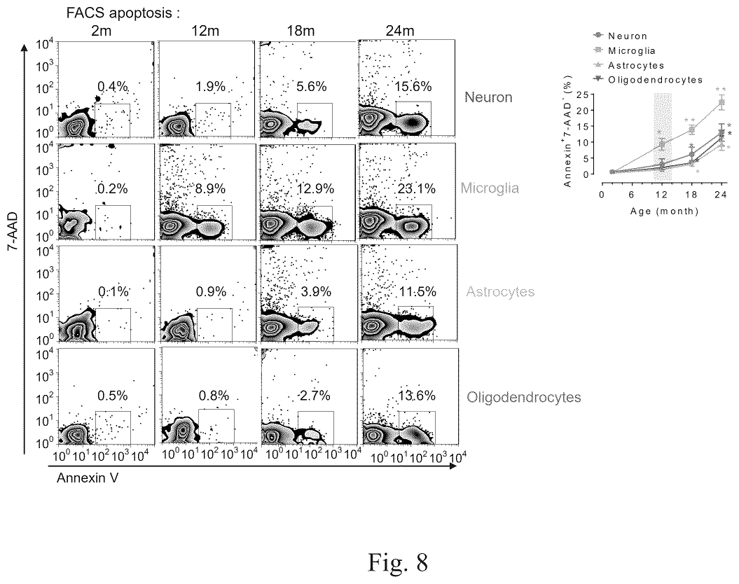

To determine whether IAA and CA have direct effects on the induction of apoptosis of microglial cells, the microglial cells were isolated from mice brains. We found that adding IAA and CA to the ex vivo cultured microglial cells isolated from young mice led to increasing the cleaved caspase-3 and cleaved poly(-ADP-ribose) polymerase (PARP) (FIG. 2E). The effect of IAA and CA on the microglia was abolished in S100A8 deficient microglia cells (FIG. 11). FACS analysis revealed a significant higher percentage of Annexin-FJTC+ apoptotic bodies (ABs) in the supernatants of cultured FACS sorted microglial cells from aged mice when compared with supernatants from cultured microglial cells from young mice (FIG. 2F). Adding IAA and CA to microglial cells isolated from young mice led to an increase in the ABs released into the culture supernatants (FIG. 2F).

### Example 2

### Reduction of Gut Bacteriophages Contributes to the Overproduction of Gut Bacterial IAA and CA

To determine whether specific bacteria contribute to the apoptosis of microglia in aging, the composition of gut bacteria in humans at different ages was assessed using next generation sequencing (NGS) of 16s ribosomal RNA (rRNA). We applied principal coordinates analyses (PCoA) to UniFrac distances of 16S rRNA amplicon profiles generated from fecal samples collected from young (25.6±4.2 years old, n=12) and old (65.3±5.6 years old, n=12) healthy human subjects (FIGS. 3A and 3B, Table 4).

TABLE 4
List of human gut [symbol] composition [symbol] (%)

| | Young [symbol] | | | | | | | | | | | | Aged [symbol] | | | | | | | | | | | |
|---|---|---|---|---|---|---|---|---|---|---|---|---|---|---|---|---|---|---|---|---|---|---|---|---|
| Taxonomy | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _{—} | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] _ [symbol] _ [symbol] _ [symbol] _ [symbol] _ [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

[symbol]

[symbol]

indicates data missing or illegible when filed

Differences in microbiome composition between the two groups were observed by PCoA analysis (FIG. 3B, left panel) and rRNA analysis indicates that microbiota from the aged individuals are significantly different from the young individuals (p=0.023, Wilcoxon; FIG. 3B, right panel). We did notice that the Ruminococcaceae, Clostridiaceae and Lachnospiraceae families were significantly overrepresented taxa in older people (FIG. 3A). The young people group had more Lactobacillaceae and Prevotellaceae. These results are consistent with previous human as well as qPCR analysis of selected bacteria abundance in the mouse large and small intestine (FIG. 3C). Given the crucial roles of intestinal bacteriophages (phages) in shaping gut bacterial composition, we hypothesized that phages may have an impact on their host by modulating the bacterial composition as well as their metabolites, including chemical substances that gain entry into the brain. To test our hypothesis, we first predicted the phages using Virus-Host DB that would potentially play a role in the enrichment of the bacteria Ruminococcaceae, Clostridiaceae and Lachnospiraceae in the aged murine gut. A total of 29 phage genera were found to be candidate phages that could target bacteria overproduced in the gut of elderly subjects. Six genera of Myoviridae and two genera of Siphoviridae potentially target Ruminococcaceae bacteria, while four genera of Myoviridae, nine genera of Siphoviridae and seven genera of Podoviridae potentially target Clostridiaceae bacteria (FIG. 3D). Only one unclassified Siphoviridae phage potentially targets Lachnospiraceae. Siphoviridae was the most abundant family, accounting for 55.3±9.8% of all identified phages, followed by Myoviridae (21.7±9.9%) and Podoviridae (10.6±8.4%) in human gut. We next sought to assess the phage genomic analysis with qPCR. Interestingly, in phages potentially targeting Ruminococcaceae, only phages belonging to the Myoviridae family, including the Brigitvirus, Eponavirus, but not the Siphoviridae family, were dramatically reduced in aged mouse gut (FIG. 3E), which is consistent with the induction of the putative target bacteria family Ruminococcaceae. In phages potentially targeting Clostridiaceae, although we did not find a difference in the Myoviridae family phiCT19406B or phiCDHM19 or the Siphoviridae family c-st or phi3626 between aged and young mice, the family Podoviridae CPS1 and CPS2 was detected in low levels in aged mice (FIG. 3E). The level of the Siphoviridae phage family displayed no difference between young and aged mice which agrees with data showing no difference in the level of potential Siphoviridae phage targeting bacterial Lachnospiraceae between aged and young mice (FIG. 3C). These results indicated that the Myoviridae and Podoviridae families specifically regulates the bacteria Ruminococcaceae and the Clostridiaceae family, respectively. To further demonstrate the role of the phages in the growth of their putative target bacteria, total gut bacterial phages from feces of young subjects were isolated and purified, and subsequently used for infection of Ruminococcaceae (ATCC, TSD-27) bacteria. Then, bacterial phages (phage^{TSD-27}) from infected TSD-27 were isolated and exposed to TSD-27 in cell culture or gavage-given to mice (FIG. 3F). The result suggested that growth of TSD-27 was inhibited by the human gut-derived phages that targeted the TSD-27 bacterium (FIG. 3F), along with the reduction of metabolites IAA and CC in the growth medium (FIG. 3G, left panel). The virome sequencing analysis using NGS indicated that Myoviridae are predominate in phage^{TSD-27} (FIG. 3H). Orally gavage administrated phage^{TSD-27} also resulted in the reduction of the metabolites IAA and CC in mouse gut (FIG. 3G, right panel) as well as decreased the expression of S100A8, cleaved caspase-3 (FIG. 11) and production of apoptosis bodies in microglia. Taken together, these lines of experimental evidence support two notions: (1) that reduction of the bacteriophage family Myoviridae contributes to the induction of gut microbiota families Ruminococcaceae leading to the induction of the metabolites IAA and CC in aged mice; and (2) that gut bacterial metabolites IAA induces microglia apoptosis via modulation of S100A8 expression. IAA induces the expression of an aging related sensome including S100A8 in microglia, eventually resulting in the microglia activation and apoptosis.

### Example 3

### IAA Prebinding is Required for p53 to Access the S100A8 Promoter Region by Unwinding the Promoter Hairpin Structure of S100A8

Given induction of bacterial metabolite IAA causes the degeneration of microglia by elevation of S100A8 at both the transcription and protein level, we hypothesized that IAA might directly target the promoter of S100A8 and regulate the expression of S100A8. Studying the interaction of metabolites, particularly small molecules, with nucleic acid is challenging due to a lack of appropriate tags for the metabolites. Because of this challenge, we developed a strategy raising the possibility to investigate the interaction of small molecules and nucleic acid. Considering that the single strand nucleic acid chain exhibits conformational differences on nondenaturing polyacrylamide gel electrophoresis (PAGE), we assumed that single-strand DNA will display an electrophoretic mobility shift on native PAGE when the small molecules bind to DNA, which is named as single-strand gel shift (SSGS; FIG. 4A).

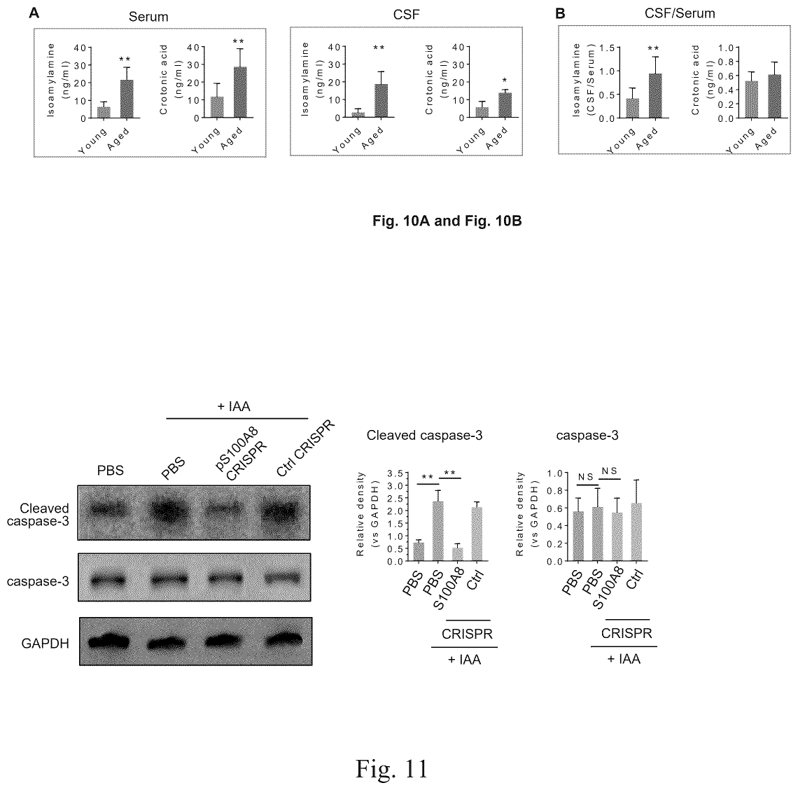

To test our hypothesis, we first synthesized short single-DNA strains (around 60 mer length each) whose sequences spanned the promoter of S100A8 (FIG. 13A and Table 5). The site and sequence of the promoter and transcription start site (TSS) were determined according to the entire mRNA (Accession #NC_000069.7 (90,576,378 . . . 90,577,341)) and genomic sequence (Accession #L76381) from the NCBI nucleic acid database (FIG. 13A). These DNA oligos (2 μM) were incubated with IAA (10 μM) following native PAGE analysis (FIG. 4A). Interestingly, we found that only oligo S100p1 (−57 to −1 upstream from the TSS) was mobility shifted by IAA or IAA enriched fecal supernatant from aged mice (FIG. 4B, FIG. 13B). To define the accurate binding site of IAA, we synthesized 10 mer length DNA oligos covering the sequence of S100p1 (Table 5). Unfortunately, the 10 mer length oligo lost the conformational mobility shift in PAGE (FIG. 13C). We then tested the mobility shift with 20 mer oligos. Only oligo S100p1-G displayed a shift (FIG. 4C, FIG. 13D), which indicated that the sequence of S100p1-G contained a potential essential sequence for metabolite IAA binding. To further verify the dynamic interaction of IAA and oligo S100p1-G, we next performed surface plasmon resonance (SPR) analysis, which is an optical technique for detecting the interaction of two different molecules in which a biotin labeled oligo is immobilized on a streptavidin sensor chip while the IAA is brought into contact via the flow cells of an SPR instrument. The SPR assay indicated that biotin labeled (Bio-) S100p1-G and S100p1, rather than mutant oligo (S100p1-GM), were targeted by IAA (FIG. 4D) which is consistent with the observation from the SSGS assay. To assess the activity of this physical binding, we constructed the 1,000 nt of promoter sequence into a luciferase reporter pGLuc. The mutant (pGLuc-S100A1-GM) was used as a control (FIG. 4C). The luciferase assay demonstrated that IAA activated the promoter sequence of S100A8 but not the mutant (FIG. 4E). To further reveal the specific DNA motif necessary for IAA binding, we synthesized 20 mer length DNA oligos that contained the potential IAA binding motif. Among the 20 oligos, each oligo contained one mutated base. We assumed that the IAA binding activity of an oligo would diminish if an essential base was replaced. As expected, the SSGS analysis suggested that the sequence TGGnCAGCTGnCCA (SEQ ID NO: 278), −41 to −54 upstream of the S100A8 TSS was necessary for IAA binding (FIG. 4F).

This finding was significant in that it provided a basis for developing mechanism-driven novel approaches for studying gut metabolites as signaling conduits that interconnect extrinsic microbial presence with activity of brain microglial cells and provided the foundation for further studying whether other gut microbiome derived small molecules are also capable of regulating the activity of transcription machinery in general.

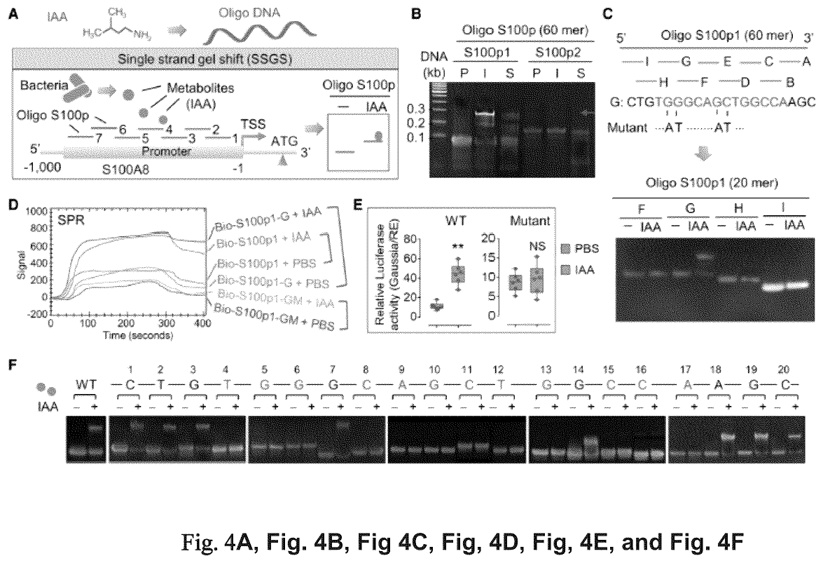

Next, we addressed how a small molecule like IAA could activate the expression of S100A8. The prediction of transcription factor binding sites using PROMO 3.0 indicated that the binding sequence resides at a p53 binding motif (GGGCAGC). Although IAA activates the transcription of S100A8 (FIG. 4E), p53 deficiency attenuated the IAA-induced S100A8 activation (FIG. 5A). However, the effect of IAA on the S100A8 transcription was recovered with additional p53 (FIG. 5B). More interestingly, further analysis demonstrated that this IAA binding motif containing a p53 binding site is a self-complementary sequence that potential curls back onto itself to form a hairpin structure (FIG. 5B). These characteristics of the IAA binding motif have allowed us to develop a hypothesis regarding the role of IAA in the transcription activation of S100A8. Transcription factor p53 cannot bind and activate S100A8 due to the hairpin structure until IAA recognizes and binds to the S100A8 promoter region. IAA binding to the promoter of S100A8 interrupts the formation of the hairpin structure and exposes the binding motif to p53, leading to transcription activation (FIG. 5B). A DNA unwinding assay further indicated that IAA enhanced the activity of helicase unwinding of the self-complementary binding sequence (FIG. 5C, left panel). Activation that promoted unwinding of mutant sequence was attenuated by IAA (FIG. 5C, right panel). The interaction of IAA and promoter of S100A8 genomic DNA was demonstrated by the chromatin immunoprecipitation (ChIP) assay as described (FIG. 5D). Additional analysis of the SPR demonstrated that p53 binds to promoter of S100A8 in an IAA dependent manner (FIG. 5E). The results generated from test tube experiments was further demonstrated in live cells. Microglial cells were treated with gut fecal supernatant and Bio-S100p1-G (Bio-WT) or Bio-S100p1-G mutant (Bio-Mutant) for 6 h and the complex was pull-downed from the cell lysate with streptavidin beads for quantitative analysis of metabolites. The LC-MS result suggest that IAA was recruited to the complex by the S100p1-G rather than the mutate of S100p1-G (FIG. 5F). Western blot analysis suggested that S100A8 induced by IAA was p53 dependent (FIG. 5G, FIG. 13E). To further identify the site and structure of molecular binding between IAA and S100p1-G, the hairpin structure of oligo S100p1-G was tested using Mfold (Jeddi and Saiz, 2017; Zuker, 2003) (FIG. 5H), followed by IAA and S100p1-G docking using the HDOCK Server. The potential mode of molecular interaction was analyzed using UCSF Chimera. The result indicated the interaction between IAA and S100p1-G at positions G-4 and G-11 takes place via two hydrogen bonds with a docking score of −39.98 and ligand rmsd (A°) of 19.83 (FIG. 5H). Multiple sequence alignment using CLUSTAL 2.1 suggested that this IAA binding motif is a conserved sequence since divergent species including human (Homo sapiens, M21005.1), mouse (Mus musculus, L76381), gorilla (Gorilla gorilla, NC_044602.1) and bovine (Bos taurus, JX070094) share this consensus sequence in the promoter region of the S100A8 gene (FIG. 5I). However, horse (Equus caballus, NC_009148.3) and aardvark (Orycteropus afer, NW_006921804.1) have a deficient IAA binding motif. To demonstrate the evolutionary relationship of this IAA binding motif, we next constructed phylogenetic trees for the six species in mammalian, based on the segment of S100A8 promoter sequences by using r package “ape” in the R 4.0 environment. The genes from Primates (human and gorilla) and Glires (mouse) share the consensus sequence to which IAA binds. However, although they share the same superorder Laurasiatheria, the order of Artiodactyla (cow) contains the IAA binding motif and the order of Perissodactyla (horse) is deficient for the IAA binding motif (FIG. 5J). SSGS analysis revealed that IAA binds to the oligo of human S100A8 sequence (M21005.1) containing the consensus binding motif as well as a few divergent flank bases (FIG. 13F, Table 5).

### Example 4

### IAA Promotes Cognitive Decline Whereas Phage^{TSD-27} Reverses Cognitive Decline in a Mouse Model

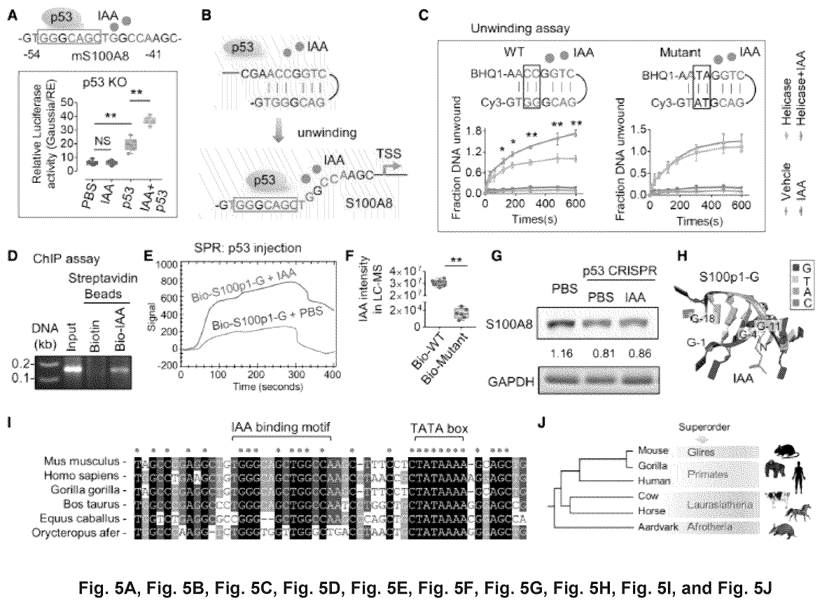

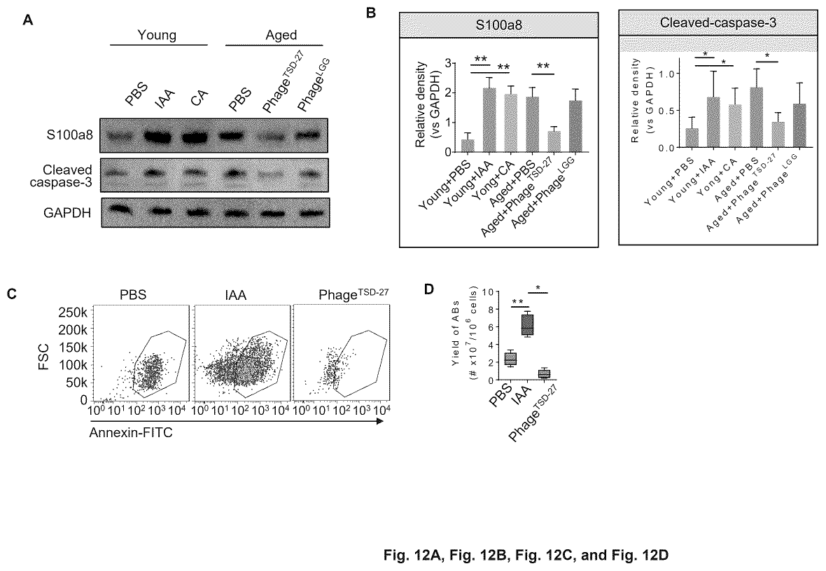

To ascertain the effect of IAA or Ruminococcaceae targeting phage on microglia and brain function in vivo, the young or aged mice were treated with IAA or phage^{TSD-27} respectively, for two months via oral gavage prior to behavioral testing (FIG. 6A). The analysis of IAA in serum using HPLC indicated that gavage-given IAA increased IAA levels in the serum and gavage-given phage^{TSD-27} significantly decreased the level of IAA in the serum of aged mice (FIG. 14A). To estimate the memory and learning in mice, we performed three behavioral and cognitive assays which included the Morris water maze (MWM), T maze spontaneous alternation (TMSA) and two-object novel object recognition (NOR). 2 month-old (young) and 12 month-old (aged) C57BL/6 mice were pretreated with IAA (0.5 g/kg of body weight) or phage^{TSD-27} (1×10⁹ pfu/mouse) every other day by oral gavage for two months prior to performing the three behavioral and cognitive assays. All subjects were trained for four consecutive days before formal testing (FIG. 6A). To test spatial learning with MWM, both young and aged mice were given a series of daily trials using five random start locations, north (N), northwest (NW), west (W), southwest (SW) and south (S) around the perimeter of the tank in the MWM (FIG. 6B). The first three trials of training were measured, and swim speeds declined noticeably in untreated aged mice compared to young mice (28.6±5.9 cm/s vs 20.0±4.1 cm/s, p=0.006) (FIG. 14B). However, IAA treatment reduced the swim speeds of young mice and phage treatment improved the swim speeds of aged mice. FIG. 6C shows the swim-paths of representative animals for the trial for the W and S locations for each group. The quantification of swim path indicated that the aged mice spent more latency time to reach the platform in all start locations (Vorhees and Williams, 2006), and IAA induced and phage reduced the latency time of young and aged mice, respectively (FIG. 6D, left panel). Some studies use swim time that animals navigate toward the hidden platform. However, to avoid the bias of different swim speeds, we normalized the test path length for each mouse based on the initial trial path length as a better way to estimate the ability to learn. The results suggested that IAA induced the memory and learning loss of young mice and phage improved the memory and learning of aged mice (FIG. 6D, right).

To verify the finding in the MWM test, we next estimated the cognitive ability of mice using the TMSA. For the purposes of analysis, the percentage of correct choices was used to distinguish the difference between aged mice and young mice as well as treatment groups (FIG. 6E, left). Consistently, IAA induced the deficit of spatial learning in the TMSA in young mice, and phage treatment attenuated the deficit in aged mice (FIG. 6E, right). The percentage of alternating choices showed that aged mice prefer to visit a familiar arm of the maze rather than a new arm. IAA administration reduced young mice exploring the new path. On the contrary, phage^{TSD-27} administration to the aged mice resulted in the mice exploring the new arm, which indicated that the TMSA was an efficient behavioral test for measuring the willingness to explore and evaluate memory a new environment. Many regions of the brain including the hippocampus, septum, basal forebrain, and prefrontal cortex are involved in this task. NOR is a nonstressful task and an appropriate means to assess memory updating for both young and aged rodents (FIG. 6F, left). The result of NOR suggested that phage administration resulted in and proved beneficial for improving age-related cognitive decline (FIG. 6F, right). The analysis of histology in the brain indicated that IAA significantly promoted the neuronal loss and dying neurons in the brain of young and phageTSD-27 attenuated the neuronal loss and dying neurons in the brain of aged mice, especially in the cerebrum and cerebellum (FIG. 6G).

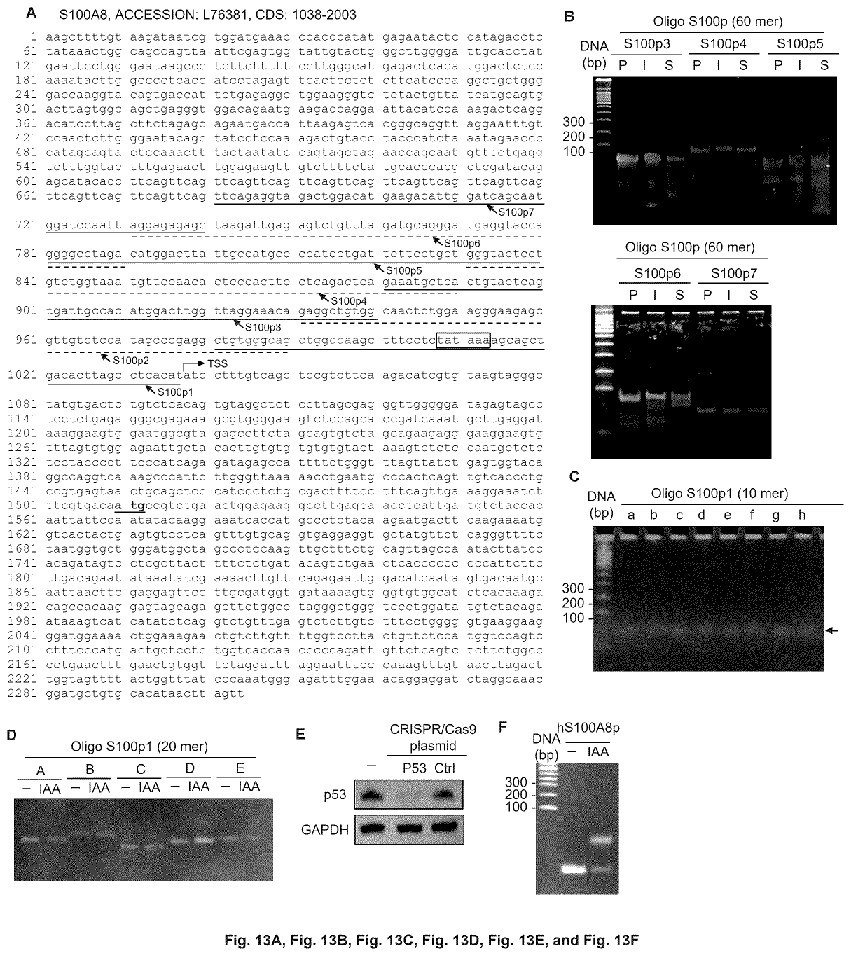

Electroencephalography (EEG) is widely utilized for behavioral and pharmacological studies in animal models as well as in humans because it is noninvasive, provides high performance, and is convenient. To determine whether EEG phenotypes between young and aged mice are different and if the IAA or phage treatment influences EEG phenotypes, we captured the epidural EEG from multiple cortical surface loci in mice using a modified method. By designing an epidural EEG electrode, we simplified the surgical procedure to minimize brain damage and optimized the experimental setup for EEG in mice (FIG. 15). The epidural EEG and the analysis of the density spectral assay (DSA) power presented distinctive waveforms and distribution of EEG strength over time between treatment and control of mice (FIG. 6H). EEG patterns in IAA mice exhibited an incoherence in the EEG rhythms compared with the EEG in control mice. Phage administration to aged mice resulted in a recovery of the coherence (FIG. 6H, left). EEG frequency is usually described in terms of five frequency bands: s (below 4 Hz), q (4-8 Hz), a (8-12 Hz), b (12-22 Hz), and g (beyond 22 Hz). Based on the quantification of wave frequency, we found that the aged mice showed a shift toward less fast a and b waves but slower s waves in the frontal and occipital lobes when comparing young mice and aged mice. Phage administration to aged mice reversed the alternation of wave frequency (FIGS. 6H, left and 6I). More interesting, the distributions of DSA in aged mice and IAA-treated young mice were asymmetric bilaterally, but young mice and phage-treated aged mice were more symmetric bilaterally. The occipital lobes on both the left and right sides exhibited higher DSA power when comparing with the frontal lobes of the brain on the same animal, as well as the corresponding lobes of the young mice. Phage significantly eliminated the induction of DSA in the occipital regions in the brain of aged mice (FIG. 6H, right). The analysis of gene expression indicated that IAA induced S100A8 and cleaved caspase-3 in the microglia from all five major regions of the brain (FIG. 6J). Collectively, these data suggested that IAA promoted memory and learning loss, whereas the phage^{TSD-27} reversed IAA-mediated memory and learning loss.

### Example 5

### IAA Plays a Causative Role in Cognitive Decline

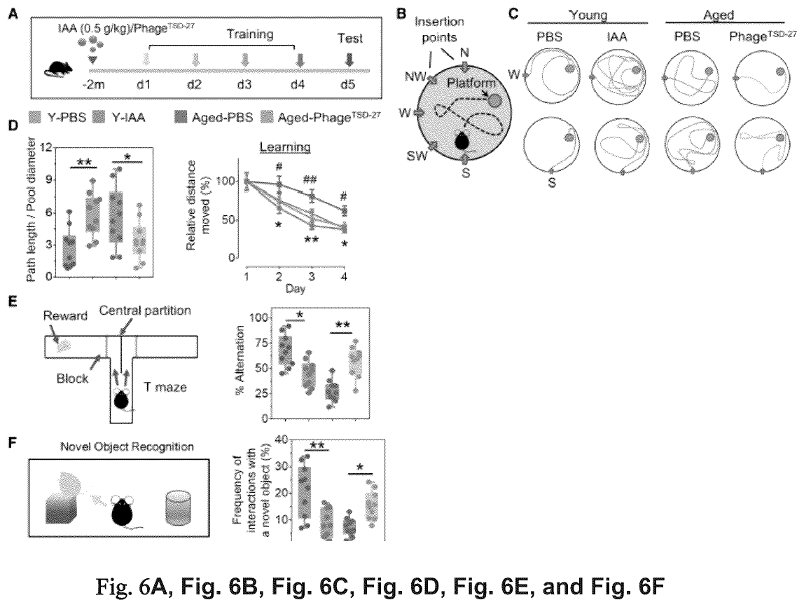

Next, we determined whether depletion of gut bacterial metabolite IAA would prevent gut metabolite mediated induction of cognitive dysfunction. Given that S100p1-G can specifically bind to IAA, we hypothesized that IAA from gut fecal supernatant could be depleted with oligo S100p1-G (FIG. 7A), leading to less IAA targeting to the promoter of S100A8. As a result of this manipulation, IAA mediated activation of microglial cells would be prevented via the S1008A mediated pathway.

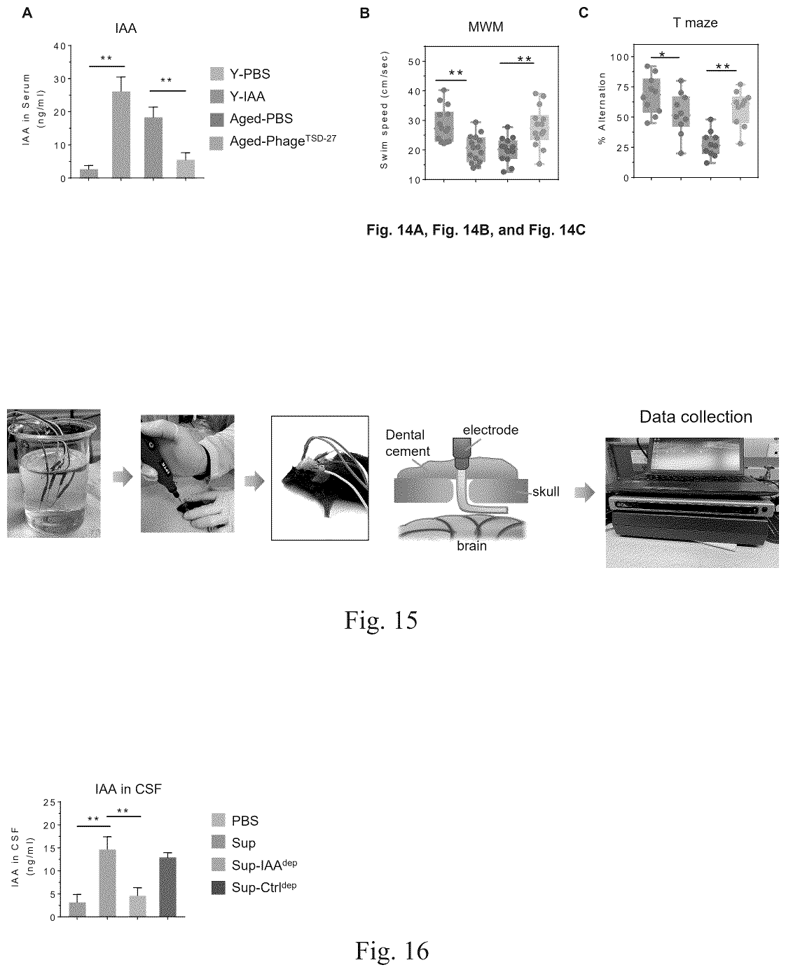

Beyond verifying the biological role of IAA in induction of cognitive dysfunction, S100p1-G-based therapy may be beneficial for improving or reversing the cognitive decline. To estimate the stability of small DNA oligo in stomach and intestine, the 20 nt oligo S100p1-G was exposed to gastric and intestine juice collected from mouse for 3 h. The electrophoresis indicated that the oligo is stable in gastric and intestine juice. This result is also supported by others. HPLC analysis suggested that the majority of the IAA (78.4%±8.2%) in the gut supernatant (Sup) of aged mice could be depleted by biotin-S100p1-G pull-down using streptavidin beads (IAAdep) but not by control S100p1-G mutant (Ctrldep) (FIG. 7B, left). Gut supernatant of aged mice gavage given to young mice leads to increasing the IAA levels in serum (FIG. 7B, right) as well as in CSF (FIG. 16) in 24 h. The depletion of IAA in aged mice-derived gut supernatants abolished the increase of IAA in young mice. Next, to determine the effect of oligo S100p1-G on the memory loss caused by Ruminococcaceae-derived IAA, the Ruminococcaceae family bacteria TSD-27 was administered to young mice along with TSD-27 targeting phage and S100p1-G. The Lactobacillus rhamnosus GG (LGG) binding phage and S100p1 mutant were used as controls. The brain memory and cognitive assay MWM (FIG. 7C) and TMSA (FIG. 7D) suggested that TSD-27 increased the latency time and swim path to find the platform, as well as reduced young mice exploring the new path. These effects of TSD-27 on the activity of brain memory and cognitive function were abolished by phage^{TSD-27} but not phage^{LGG}. The fecal supernatant of aged mice showed that memory and learning loss is IAA dependent (FIGS. 7C and 7D). Aged mice IAA level analysis confirmed that gavage-given TSD-27 increased the IAA level, and phage^{TSD-27} canceled the effect of TSD-27 on the increasing IAA (FIG. 7E). Collectively, these results suggest that phage^{TSD-27} can prevent gut bacterial IAA-induced learning and spatial learning loss. We further identified whether the treatments as described above alters the microglial cell activity. Analysis by qPCR (FIG. 7F) and western blot (FIG. 7G) indicated that gavage-given TSD-27 and aged supernatant induce the expression of S100A8 and more apoptotic cleaved caspase-3 induced in microglia cells; phageTSD-27 and IAA depletion with S100p1-G prevented these TSD-27 and aged supernatant-induced effects.

From a therapeutic standard point, we next sought to determine whether oligo S100p1-G could reverse the brain cognitive dysfunction in aged mice by blocking IAA. S100p1-G was administered to aged mice via an intravenous route twice a week for 2 months. Memory and spatial learning were recovered by treatment with S100p1-G (FIG. 7H) along with a decline of IAA in CSF of mice (FIG. 7I); these results were not seen with the mutant S100p1-G. Collectively, our data suggest that the induction of Ruminococcaceae due to reduction of its phages leads to an increase of the bacterial metabolite IAA. In turn, IAA joins with TF p53 to induce the expression of the proinflammatory and pro-apoptosis S100A8 in microglial cells by binding the promoter motif of S100A8. The activation of microglial cells contributes to the cognitive dysfunction in aged mice. S100p1-G therapy has the potential to reverse the IAA-mediated memory and learning losing in aging.

### Discussion of the Examples

Despite advances in metabolic techniques, a complete portrait of the metabolism and its complement of dynamic interactions with DNA remains unrealized. Specifically, traditional biological and analytical approaches have not been able to address key questions relating to the interactions of DNA with small molecules, including drugs, drug candidates, and metabolites. The introduction of a simple and effective strategy such as the SSGS technology we developed in this study in combination with other metabolic technologies to parse and enrich subsets of the “functional” small molecules that bind to DNA will allow scientists to delve more deeply and precisely into the gene expression state of cells. Additionally, this technology will facilitate researchers to explore perturbations by small molecules to overcome limitations of biological approaches to enable the selective marking and functional investigation of critical DNA-small-molecule interactions in biological environments such as a gut microbiome-enriched environment.

Recent advances in metabolic technologies have greatly enhanced our understanding of metabolites as a complement to the genome and have provided valuable insight into numerous physiological processes and disease mechanisms. However, such methods alone do not capture the myriad of post-metabolite events such as gut microbiome-derived metabolites and molecular interactions that can affect gene expression. The dramatic contextual and temporal variability of metabolites is part of what sets it apart from the relatively stable genome, limiting the reach of genome-based strategies to study metabolites and their interactions. Our approach as described in this study provides a foundation for further studying the mechanism underlying how temporal variability of the metabolites, such as gut metabolites generated under physiological versus pathophysiological conditions, has an effect on gene expression.

Traditional biological approaches, including the genetic incorporation of affinity tags and the creation of selective antibodies, have been transformative for the detection of protein-protein interactions. However, this approach does not apply to small molecules because unlike proteins that are relatively large, when a tag is incorporated into a small molecule, like a drug or metabolite, the tag likely perturbs the ability of the small molecule to interact with DNA. Investigating small molecules without labeling them as with the SSGS technology we developed in this study has bridged this experimental gap to demonstrate small molecule binding to DNA. This technology allows for the screening of many small molecules that potentially bind to DNA in a DNA sequencing-specific manner for downstream investigations.

With this technology, it was discovered that IAA and p53 jointly activate S100A8 signaling of microglia leading to cognitive decline in aged mice. p53, a TF discovered 40 years ago, is one of the most extensively studied genes (Levine and Oren, 2009) and has previously been implicated in neurodegeneration. A significant increase in (Levine and Oren, 2009) p53 levels and activity was detected in postmortem CNS tissues of patients with ALS as well as in other neurodegenerative diseases, including Alzheimer disease, Parkinson disease, and Huntington disease. As disclosed herein, we uncovered aging-dependent induction of IAA that advances p53 accessibility to the promoter region of S100A8 by shaping the hairpin structure of S100A8 promoter region.

Using IAA as a proof of concept, we have demonstrated that shifting gut metabolites can be a signaling conduit that interconnects the extrinsic microbial environment with activity of brain microglial cells and provides a foundation for further studying whether other gut microbiome-derived metabolites are also capable of regulating the activity of TFs in general.

Unlike a standard ChIP assay that generates background noises/off-target signals, with PCR technology in combination with the ChIP assay, we can not only confirm the specific interaction of S100A8 promoter with IAA, but this method also provides a strategy to investigate whether other detected signals generated from the ChIP assay are actually signals due to IAA treatment.

The present disclosure also demonstrated that specific sequencing of S100A8 promoter is required for IAA binding. The docking analysis indicated that the IAA butan-1-amine carrying a methyl substituent at position 3 is required for the recognition of the oligo S100p1-G binding motif (-NTGGNCAGCTGNCCA-) via binding to the 4th and 11th guanine base of S100p1-G with the hydrogen bonds. This docking analysis also agrees with our experimental data presented in FIGS. 4C, 4D, and 4F. Moreover, the LC-MS analysis of the Bio-S100p1-G complex from cell lysates indicated that only IAA interacts with S100p1-G (FIG. 5F), and we did not find that the other molecules including polyamines, GABA, and lysine are within the Bio-S100p1-G complex, suggesting that the S100p1-G/IAA interaction is IAA specific.

The G-rich DNA oligo is able to distinguish one atomic difference between small molecule ochratoxin analogs A (OTA) and OTB, showing specificity recognition and affinity among hundreds of aptamers for small molecules. Given the S100p1-G is G enriched, the selectivity of interaction between S100A8 and IAA could be contributed by specific tertiary (3D) structure of DNA and a high level of G enrichment.

Additionally, we also found that the microbiome-bacteriophage-metabolite axis is dysregulated during aging, leading to induction of IAA that impairs microglial cell function, contributing to exacerbated brain memory loss. In this study, we show that the IAA-mediated pathological effect can be prevented. We have demonstrated that S100p1-G oligo prevents the IAA-induced memory loss. The significance of this finding lies not only in developing personalized oligo therapy to delay aging but also holds the potential to change the therapeutic landscape for many neurological and non-neurological conditions by oligo-mediated manipulation of gut metabolite activity.

The presently disclosed subject matter focused on exploring the effects of the microbiome/bacteriophage-regulated IAA on microglial cell activation including apoptosis and inflammation, which may impact downstream neuron function in both direct and indirect manners. The pathways regulated by IAA in neuron cells and microglial cells in the later stages of aging may necessitate cross talk to have an effect on downstream brain function and inflammation.

### REFERENCES

All references listed throughout the instant disclosure, including but not limited to all patents, patent applications and publications thereof, scientific journal articles, and database entries (e.g., GENBANK® biosequence database) are incorporated herein by reference in their entireties to the extent that they supplement, explain, and/or teach methodology, techniques, and/or compositions employed herein.
Anders et al. (2014). Genome-wide localization of small molecules. Nat. Biotechnol. 32, 92-96.
Angelova and Brown (2019). Microglia and the aging brain: are senescent microglia the key to neurodegeneration? J. Neurochem. 151, 676-688.
Bae et al. (2005). p 53 mediates cellular dysfunction and behavioral abnormalities in Huntington's disease. Neuron 47, 29-41.
Baker et al. (2008). EEG patterns in mild cognitive impairment (MCI) patients. Open Neuroimag. J. 2, 52-55.
Bernard-Patrzynski et al. (2019). Isolation of endothelial cells, pericytes and astrocytes from mouse brain. PLoS One 14, e0226302.
Bolyen et al. (2019). Reproducible, interactive, scalable and extensible microbiome data science using QIIME 2. Nat. Biotechnol. 37, 852-857.
Cardona et al. (2006). Isolation of murine microglial cells for RNA analysis or flow cytometry. Nat. Protoc. 1, 1947-1951.
Chen et al. (2000). A learning deficit related to age and beta-amyloid plaques in a mouse model of Alzheimer's disease. Nature 408, 975-979.
Cho et al. (2016). Older age results in differential gene expression after mild traumatic brain injury and is linked to imaging differences at acute follow-up. Front. Aging Neurosci. 8, 168.
Chu et al. (2019). The microbiota regulate neuronal function and fear extinction learning. Nature 574, 543-548.
Clark et al. (2021). Cerebellar-subcortical-cortical systems as modulators of cognitive functions. Neuropsychol. Rev. 31, 422-446.
Cristóvão and Gomes (2019). S100 proteins in Alzheimer's disease. Front. Neurosci. 13, 463.
de la Monte et al. (1997). Correlates of p53- and Fas (CD95)-mediated apoptosis in Alzheimer's disease. J. Neurol. Sci. 152, 73-83.
Deacon and Rawlins (2006). T-maze alternation in the rodent. Nat. Protoc. 1, 7-12.
Deal and Henikoff (2011). The INTACT method for cell type-specific gene expression and chromatin profiling in Arabidopsis thaliana. Nat. Protoc. 6, 56-68.
DeSantis et al. (2006). Greengenes, a chimera-checked 16S rRNA gene database and workbench compatible with ARB. Appl. Environ. Microbiol. 72, 5069-5072.
Doulberis et al. (2019). Microbes and Alzheimer' disease: lessons from H. pylori and GUT microbiota. Eur. Rev. Med. Pharmacol. Sci. 23, 1845-1846.
Festing and Altman (2002). Guidelines for the design and statistical analysis of experiments using laboratory animals. ILAR J. 43, 244-258.
Fujita and Yamashita (2021). Neuroprotective function of microglia in the developing brain. Neuronal Signal. 5, NS20200024.
Gouda (2015). Common pitfalls in reporting the use of SPSS software. Med. Princ. Pract. 24, 300.
Hale et al. (2020). Cognitive impairment in the U.S.: lifetime risk, age at onset, and years impaired. SSM Popul. Health 11, 100577.
Hemonnot et al. (2019). Microglia in Alzheimer disease: well-known targets and new opportunities. Front. Aging Neurosci. 11, 233.
Hickman et al. (2013). The microglial sensome revealed by direct RNA sequencing. Nat. Neurosci. 16, 1896-1905.
Holwerda and de Laat (2013). CTCF: the protein, the binding partners, the binding sites and their chromatin loops. Philos. Trans. R. Soc. Lond. B Biol. Sci. 368, 20120369.
Hristov et al. (2004). Apoptotic bodies from endothelial cells enhance the number and initiate the differentiation of human endothelial progenitor cells in vitro. Blood 104, 2761-2766.
Hu and Lin (2021). S100a8 silencing attenuates inflammation, oxidative stress and apoptosis in BV2 cells induced by oxygen-glucose deprivation and reoxygenation by upregulating GAB1 expression. Mol. Med. Rep. 23, 64.
Hu et al. (2021). Evolution of DNA methylome from precancerous lesions to invasive lung adenocarcinomas. Nat. Commun. 12, 687.
Huang et al. (2014). Enhancing UCSF Chimera through web services. Nucleic Acids Res. 42, W478-W484.
Jeddi and Saiz (2017). Three-dimensional modeling of single stranded DNA hairpins for aptamer-based biosensors. Sci. Rep. 7, 1178.
Kadosh et al. (2020). The gut microbiome switches mutant p53 from tumour-suppressive to oncogenic. Nature 586, 133-138.
Kim and Joh (2006). Microglia, major player in the brain inflammation: their roles in the pathogenesis of Parkinson's disease. Exp. Mol. Med. 38, 333-347.
Kwapis et al. (2020). Aging mice show impaired memory updating in the novel OUL updating paradigm. Neuropsychopharmacology 45, 337-346.
Langille et al. (2014). Microbial shifts in the aging mouse gut. Microbiome 2, 50.
Larkin et al. (2007). Clustal W and Clustal X version 2.0. Bioinformatics 23, 2947-2948.
Le Boulch et al. (2019). The MACADAM database: a MetAboliC pAthways DAtabase for Microbial taxonomic groups for mining potential metabolic capacities of archaeal and bacterial taxonomic groups. Database (Oxford) 2019, baz049.
Leger et al. (2013). Object recognition test in mice. Nat. Protoc. 8, 2531-2537.
Levine and Oren (2009). The first 30 years of p53: growing ever more complex. Nat. Rev. Cancer 9, 749-758.
Liberti and Engel (2020). The gut microbiota-brain axis of insects. Curr. Opin. Insect Sci. 39, 6-13.
Liu et al. (2000). Discrete mobility of singlestranded DNA in non-denaturing gel electrophoresis. Nucleic Acids Res. 28, 940-943.
Liu et al. (2015). Digestion of nucleic acids starts in the stomach. Sci. Rep. 5, 11936.
Lodeiro et al. (2017). Aggregation of the inflammatory S100A8 precedes Abeta plaque formation in transgenic APP mice: positive feedback for S100A8 and Abeta productions. J. Gerontol. A Biol. Sci. Med. Sci. 72, 319-328.
Lu and Salzberg (2020). Ultrafast and accurate 16S rRNA microbial community analysis using Kraken 2. Microbiome 8, 124.
Luong et al. (2020). Standardized bacteriophage purification for personalized phage therapy. Nat. Protoc. 15, 2867-2890.
Ma et al. (2018). A human gut phage catalog correlates the gut phageome with type 2 diabetes. Microbiome 6, 24.
Masvekar et al. (2019). Quantifications of CSF apoptotic bodies do not provide clinical value in multiple sclerosis. Front. Neurol. 10, 1241.
Mattei and Notter (2020). Basic concept of microglia biology and neuroinflammation in relation to psychiatry. Curr. Top. Behav. Neurosci. 44, 9-34.
McCarrey et al. (2016). Sex differences in cognitive trajectories in clinically normal older adults. Psychol. Aging 31, 166-175.
Mihara et al. (2016). Linking virus genomes with host taxonomy. Viruses 8, 66.
Morais et al. (2021). The gut microbiota-brain axis in behaviour and brain disorders. Nat. Rev. Microbiol. 19, 241-255.
Nardone et al. (2021). TMS-EEG co-registration in patients with mild cognitive impairment, Alzheimer's disease and other dementias: A systematic review. Brain Sci. 11.
Narlikar et al. (2002). Cooperation between complexes that regulate chromatin structure and transcription. Cell 108, 475-487.
Niraula et al. (2017). Microglia priming with aging and stress. Neuropsychopharmacology 42, 318-333.
Pruesse et al. (2007). SILVA: a comprehensive online resource for quality checked and aligned ribosomal RNA sequence data compatible with ARB. Nucleic Acids Res. 35, 7188-7196.
Ren et al. (2021). COVID-19 immune features revealed by a large-scale single-cell transcriptome atlas. Cell 184, 1895-1913.e19.
Riva et al. (2012). Induction of nuclear factor-kB responses by the S100A9 protein is Toll-like receptor-4-dependent. Immunology 137, 172-182.
Sausset et al. (2020). New insights into intestinal phages. Mucosal Immunol. 13, 205-215.
Sgritta et al. (2019). Mechanisms underlying microbial-mediated changes in social behavior in mouse models of autism spectrum disorder. Neuron 101, 246-259.e6.
Shen et al. (2016). SeqKit: a cross-platform and ultrafast toolkit for FASTA/Q file manipulation. PLoS One 11, e0163962.
Simard et al. (2014). Human S100A9 potentiates IL-8 production in response to GM-CSF or fMLP via activation of a different set of transcription factors in neutrophils. FEBS Lett. 588, 2141-2146.
Swindell et al. (2013). Robust shifts in S100a9 expression with aging: a novel mechanism for chronic inflammation. Sci. Rep. 3, 1215.
Szybińska and Leśniak (2017). P53 dysfunction in neurodegenerative diseases—the cause or effect of pathological changes? Aging Dis. 8, 506-518.
Tanti et al. (2019). Isolation, culture and functional characterization of glia and endothelial cells From adult pig brain. Front. Cell. Neurosci. 13, 333.
Teng et al. (2017). MVP-mediated exosomal sorting of miR-193a promotes colon cancer progression. Nat. Commun. 8, 14448.
van Exel et al. (2001). Cognitive function in the oldest old: women perform better than men. J. Neurol. Neurosurg. Psychiatry 71, 29-32.
Vardevanyan et al. (2003). The binding of ethidium bromide with DNA: interaction with single- and double-stranded structures. Exp. Mol. Med. 35, 527-533.
Virgilio et al. (2018). Gastric juice microRNAs as potential biomarkers for screening gastric cancer: a systematic review. Anticancer Res. 38, 613-616.
Vogl et al. (2018). Autoinhibitory regulation of S100A8/S100A9 alarmin activity locally restricts sterile inflammation. J. Clin. Invest. 128, 1852-1866.
Vorhees and Williams (2006). Morris water maze: procedures for assessing spatial and related forms of learning and memory. Nat. Protoc. 1, 848-858.
Wang et al. (2018). S100A8/A9 in inflammation. Front. Immunol. 9, 1298.
Wang et al. (2015). Characterization of spatio-temporal epidural event-related potentials for mouse models of psychiatric disorders. Sci. Rep. 5, 14964.
Weil et al. (2019). Isolation and culture of oligodendrocytes. Methods Mol. Biol. 1936, 79-95.
Xu et al. (2022). Structural insights into the mechanism of high-affinity binding of ochratoxin A by a DNA aptamer. J. Am. Chem. Soc. 144, 7731-7740.
Zengeler and Lukens (2021). Innate immunity at the crossroads of healthy brain maturation and neurodevelopmental disorders. Nat. Rev. Immunol. 21, 454-468.
Zuker (2003). Mfold web server for nucleic acid folding and hybridization prediction. Nucleic Acids Res. 31, 3406-3415.
Zuo et al. (2018). Bacteriophage transfer during faecal microbiota transplantation in Clostridium difficile infection is associated with treatment outcome. Gut 67, 634-643.

It will be understood that various details of the presently disclosed subject matter can be changed without departing from the scope of the presently disclosed subject matter. Furthermore, the foregoing description is for the purpose of illustration only, and not for the purpose of limitation.

### EXEMPLARY SEQUENCES

Murine S100A8 Promoter: −357 to −1 from
Transcription Start Site (TSS; SEQ ID NO: 12):
ttcagaggtagactggacatgaaggcattggatcagcaatggatc
caattaggagagggttaagattgagagtctgtttagatgcaggga
tgaggtgccaggggcctagacatggacttattgccatgccccatc
ctgattcttcctgctgggtactcctgtctggtaaatgttccaaca
ctcccacttcctcagactcagaaatgctcactgtactcagtgatt
gccacatggacttggttaggaaacagaggctgtggcaactctgga
agggaagagcgttgtctccatagcccgaggctgtgggcagctggc
caagctttcctctataaaagcagctgacacttagcctcacat
NOTE:
the IAA binding site is presented in bold
and underlined
Human S100A8 Promoter (SEQ ID NO: 13):
tggggagaggatttgttcctcctgaaatcctggggaattggccac
ctcctcttctcctcttaggcatgaagcgcgtctggcttctccaaa
gaactcttcccctccactacctcagagttagcttcctctcttcag
ccagtgatcctggggtcccagacacaataattaaccaagagaggg
tgaaaggctccctgctgtgtttatgcaatggctcaggcccttgtg
aagtgccgagggaccccaagcagcctccatctcccagggcatggt
ccatccccagcttcacagaacaggaaagctgtggaggagtgtggg
cagcagggtaggaatggatatagcccttggcaacaacacatttcc
ccacaaagcacccacccaaaagaacaacaacgatagttttagttt
ttagtaatgagaacaatagttctcatgactaaaagccatcagcca
ggacacgtgttctcaacccttttgcggtctttggaccctttgaaa
ctctgacagaagccatggaggaatgttctcactgagtgcatgcac
tcaaaatgatgcattcaacttcaattcagtttcagggatgtatgg
cctgaccaccaatgcaggggattagcaatcgcaatagtggagagg
gcatgggagtgggaatctggctggatcaagcaagtggatgccagc
agcccagaaaaagagcccccctacctgctttttccttctgggcac
tattgcccagcaaatgccttcctctttccgcttctcctacctccc
cacccaaaattttcattctgcacagtgattgccacattcacctgg
ttgagaaaccagagactgtagcaactctggcagggagaagctgtc
tctgatggcctgaagctgtgggcagctggccaagcctaaccgcta
taaaaaggagctgcctctcagccctgcatgtctcttgtcagctgt
ctttcagaagacctggtaagtgggactgtctgggttggccccgca
ctttgggcttctcttggggagggtcagggaagtggagcagccttc
ctgagagaggagagagaaagctcagggaggtctggagcaaagata
ctcctggaggtggggagtgaggcagggataaggaaggagagtatc
ctccagcaccttccagtgggtaagggcacattgtctcctaggctg
gacttttcttgagcagagggtggggtggtaaggaaagtctacggg
ccccgtgtgtgtgcacatgtctctgtgtgaatggacccttcccct
tcccacacgtgtatccctatcatcccacccttcccaccagagcca
tagccatctgctggtttggttatttggagagtgcaggccaggaca
aggccatcgcttggggcatgaatcctctgcgtactgccctggcca
gatgcaaattccctgccatgggattccccagaaggttctgttttt
caggtggggcaagtccgtgggcatcatgttgaccgagctggagaa
agccttgaactctatcatcgacgtctaccacaagtactccctgat
aaaggggaatttccatgccgtctacagggatgacctgaagaaatt
gctagagaccgagtgtcctcagtatatcagggtgaggaggggctg
ggtgtggcgggggctctctgcctggtcctggggctgccctgggcc
agcggtcctccctgccacccttcatagatggctatgcctcggctc
tctctgagatctttaaactctggcttcttcctcctcaatcttgac
agaaaaagggtgcagacgtctggttcaaagagttggatatcaaca
ctgatggtgcagttaacttccaggagttcctcattctggtgataa
agatgggcgtggcagcccacaaaaaaagccatgaagaaagccaca
aagagtagctgagttactgggcccagaggctgggcccctggacat
gtacctgcagaataataaagtcatcaatacctcatgcctctctct
tatgcttttgtggaatgaggt
NOTE:
the IAA binding site is presented in bold
and underlined

What is claimed is:

1. A method of detecting the binding site of a small molecule to a target DNA sequence, the method comprising
a) obtaining a short single-strand DNA oligonucleotide of a target DNA sequence;
b) incubate DNA oligonucleotide with the small molecule creating a sample mixture; and
c) performing nondenaturing polyacrylamide gel electrophoresis (PAGE) on the sample mixture, whereby a band on the gel is revealed for the sample mixture;
wherein a shift in the band for the sample mixture relative to a control containing only the DNA oligonucleotide indicates the binding site for the small molecule to the target DNA sequence is present in the DNA oligonucleotide.

2. The method of claim 1, wherein the DNA oligonucleotide is at least 15 nucleotides in length.

3. The method of claim 2, wherein the DNA oligonucleotide is 20 nucleotides in length.

4. A method of screening for a small molecule that binds to a target DNA sequence, the method comprising
a) obtaining a short single-strand DNA oligonucleotide of a target DNA sequence;
b) incubate DNA oligonucleotide with the small molecule creating a sample mixture; and
c) performing nondenaturing polyacrylamide gel electrophoresis (PAGE) on the sample mixture, whereby a band on the gel is revealed for the sample mixture;
wherein a shift in the band for the sample mixture relative to a control containing only the DNA oligonucleotide indicates the small molecule binds the target DNA sequence.

5. The method of claim 4, wherein the DNA oligonucleotide is at least 15 nucleotides in length.

6. The method of claim 5, wherein the DNA oligonucleotide is 20 nucleotides in length.

7. A composition comprising an isoamyl alcohol (IAA) sequestration agent, wherein the IAA sequestration agent comprises an IAA binding moiety.

8. The composition of claim 7, wherein the IAA binding moiety comprises a nucleotide sequence or a peptide or polypeptide to which IAA binds.

9. The composition of claim 7, further comprising a pharmaceutically acceptable carrier, excipient, or diluent.

10. The composition of claim 9, wherein the pharmaceutically acceptable carrier, excipient, or diluent is pharmaceutically acceptable for use in a human.

11. A method for reducing and/or delaying cognitive decline associated with neurodegenerative disease in a subject in need thereof, the method comprising administering to the subject an effective amount of a composition comprising the composition of claim 7.

12. A method for sequestering isoamyl alcohol (IAA) produced by microbiota in the gut of a subject, the method comprising administering to the subject an effective amount of a composition comprising the composition of claim 7.

13. An oligonucleotide comprising, consisting essentially of, or consisting of the sequence 5′-TGGGCAGCTGGCCA-3′ (SEQ ID NO: 10), optionally comprising, consisting essentially of, or consisting of the sequence 5′-CTGTGGGCAGCTGGCCAAGC-3′ (SEQ ID NO: 11), wherein the oligonucleotide optionally further comprises a tag, optionally a biotin tag.

14. The oligonucleotide of claim 13, wherein the oligonucleotide comprises at least one modified base.

15. The oligonucleotide of claim 13, wherein the oligonucleotide further comprises a tag, optionally a biotin tag

16. A method for reducing and/or delaying cognitive decline associated with neurodegenerative disease in a subject in need thereof, the method comprising administering to the subject an effective amount of a composition comprising the oligonucleotide of any one of claim 13.

17. A method for sequestering isoamyl alcohol (IAA) produced by microbiota in the gut of a subject, the method comprising administering to the subject an effective amount of a composition comprising the oligonucleotide of claim 13.
